(19) United States
(12) Patent Application Publication
(10) Pub. No.: US 2016/0207928 A1
(43) Pub. Date: Jul. 21, 2016

(54) NOVEL COMPOUNDS FOR THE TREATMENT OF CANCER

(71) Applicant: BAYER PHARMA AKTIENGESELLSCHAFT, Berlin (DE)

(72) Inventors: Volker SCHULZE, Hohen Neuendorf (DE); Hartmut SCHIROK, Langenfeld (DE); Dirk KOSEMUND, Berlin (DE); Hans BRIEM, Berlin (DE); Benjamin BADER, Berlin (DE); Ulf BÖMER, Glienicke (DE); Antje Margret WENGNER, Berlin (DE); Gerhard SIEMEISTER, Berlin (DE); Philip LIENAU, Berlin (DE); Detlef STÖCKIGT, Potsdam (DE); Ulrich LÜCKING, Berlin (DE); Andreas SCHALL, Wuppertal (DE)

(21) Appl. No.: 14/896,450

(22) PCT Filed: Jun. 4, 2014
(86) PCT No.: PCT/EP2014/061530

§ 371 Date: Dec. 7, 2015

(30) Foreign Application Priority Data
Jun. 10, 2013 (EP) 13171171.5
Dec. 20, 2013 (EP) 13198899.0

(51) Int. Cl. C07D 487/04; A61K 31/5025; A61K 31/4985; A61K 31/437; A61K 45/06; C07D 277/82; C07D 471/04; A61K 31/5377; A61K 31/428

(52) U.S. Cl. CPC C07D 487/04; A61K 31/5377; A61K 31/5025; A61K 31/4985; A61K 31/428; A61K 45/06; C07D 277/82; C07D 471/04; A61K 31/437

(57) ABSTRACT

The present invention relates to novel compounds showing an inhibitory effect on Mps-1 kinase, to methods of preparing said compounds, to pharmaceutical compositions and combinations comprising said compounds, to the use of said compounds for manufacturing a pharmaceutical composition for the treatment or prophylaxis of a disease, as well as to intermediate compounds useful in the preparation of said compounds.

The present invention relates to novel compounds of general formula (I) as described and defined herein, to methods of preparing said compounds, to pharmaceutical compositions and combinations comprising said compounds, to the use of said compounds for manufacturing a pharmaceutical composition for the treatment or prophylaxis of a disease, as well as to intermediate compounds useful in the preparation of said compounds.

### BACKGROUND OF THE INVENTION

The present invention relates to chemical compounds that inhibit Mps-1 (Monopolar Spindle 1) kinase (also known as Tyrosine Threonine Kinase, TTK). Mps-1 is a dual specificity Ser/Thr kinase which plays a key role in the activation of the mitotic checkpoint (also known as spindle checkpoint, spindle assembly checkpoint) thereby ensuring proper chromosome segregation during mitosis [Abrieu A et al., Cell, 2001, 106, 83-93]. Every dividing cell has to ensure equal separation of the replicated chromosomes into the two daughter cells. Upon entry into mitosis, chromosomes are attached at their kinetochores to the microtubules of the spindle apparatus. The mitotic checkpoint is a surveillance mechanism that is active as long as unattached kinetochores are present and prevents mitotic cells from entering anaphase and thereby completing cell division with unattached chromosomes [Suijkerbuijk S J and Kops G J, Biochemica et Biophysica Acta, 2008, 1786, 24-31; Musacchio A and Salmon E D, Nat Rev Mot Cell Biol., 2007, 8, 379-93]. Once all kinetochores are attached in a correct amphitelic, i.e. bipolar, fashion with the mitotic spindle, the checkpoint is satisfied and the cell enters anaphase and proceeds through mitosis. The mitotic checkpoint consists of complex network of a number of essential proteins, including members of the MAD (mitotic arrest deficient, MAD 1-3) and Bub (Budding uninhibited by benzimidazole, Bub 1-3) families, the motor protein CENP-E, Mps-1 kinase as well as other components, many of these being over-expressed in proliferating cells (e.g. cancer cells) and tissues [Yuan B et al., Clinical Cancer Research, 2006, 12, 405-10]. The essential role of Mps-1 kinase activity in mitotic checkpoint signalling has been shown by shRNA-silencing, chemical genetics as well as chemical inhibitors of Mps-1 kinase [Jelluma N et al., PLos ONE, 2008, 3, e2415; Jones M H et al., Current Biology, 2005, 15, 160-65; Dorer R K et al., Current Biology, 2005, 15, 1070-76; Schmidt M et al., EMBO Reports, 2005, 6, 866-72].

There is ample evidence linking reduced but incomplete mitotic checkpoint function with aneuploidy and tumorigenesis [Weaver B A and Cleveland D W, Cancer Research, 2007, 67, 10103-5; King R W, Biochimica et Biophysica Acta, 2008, 1786, 4-14]. In contrast, complete inhibition of the mitotic checkpoint has been recognised to result in severe chromosome missegregation and induction of apoptosis in tumour cells [Kops G J et al., Nature Reviews Cancer, 2005, 5, 773-85; Schmidt M and Medema R H, Cell Cycle, 2006, 5, 159-63; Schmidt M and Bastians H, Drug Resistance Updates, 2007, 10, 162-81].

Therefore, mitotic checkpoint abrogation through pharmacological inhibition of Mps-1 kinase or other components of the mitotic checkpoint represents a new approach for the treatment of proliferative disorders including solid tumours such as carcinomas and sarcomas and leukaemias and lymphoid malignancies or other disorders associated with uncontrolled cellular proliferation.

Different compounds have been disclosed in prior art which show an inhibitory effect on Mps-1 kinase:

WO 2009/024824 A1 discloses 2-Anilinopurin-8-ones as inhibitors of Mps-1 for the treatment of proliferate disorders. WO 2010/124826 A1 discloses substituted imidazoquinoxaline compounds as inhibitors of Mps-1 kinase. WO 2011/026579 A1 discloses substituted aminoquinoxalines as Mps-1 inhibitors.

WO2011/157688(A1), WO2011/063908(A1), WO2011/064328(A1), WO2011063907(A1) and WO2012/143329(A1) disclose substituted triazolopyridine compounds as inhibitors of Mps-1 kinase.

However, the state of the art described above does not describe the compounds of general formula (I) of the present invention, or a stereoisomer, a tautomer, an N-oxide, a hydrate, a solvate, or a salt thereof, or a mixture of same, as described and defined herein, and as hereinafter referred to as “compounds of the present invention”, or their pharmacological activity. It has now been found, and this constitutes the basis of the present invention, that said compounds of the present invention have surprising and advantageous properties.

In particular, said compounds of the present invention have surprisingly been found to effectively inhibit Mps-1 kinase and may therefore be used for the treatment or prophylaxis of diseases of uncontrolled cell growth, proliferation and/or survival, inappropriate cellular immune responses, or inappropriate cellular inflammatory responses or diseases which are accompanied with uncontrolled cell growth, proliferation and/or survival, inappropriate cellular immune responses, or inappropriate cellular inflammatory responses, particularly in which the uncontrolled cell growth, proliferation and/or survival, inappropriate cellular immune responses, or inappropriate cellular inflammatory responses is mediated by Mps-1 kinase, such as, for example, haemotological tumours, solid tumours, and/or metastases thereof, e.g. leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof.

### SUMMARY OF THE INVENTION

The present invention covers compounds of general formula (I):

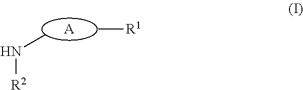

in which:
A is selected from:

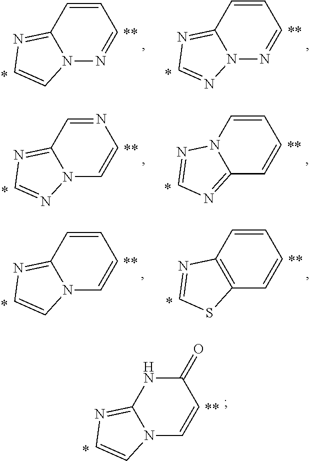

wherein * represents the point of attachment to the nitrogen atom and ** represents the point of attachment to the R¹ group;
R¹ represents a phenyl-group
   which is substituted, one or more times, identically or differently, with a substituent selected from:
   —OH, —N(H)C(═O)R⁶, —N(R⁷)C(═O)R⁶, —N(H)C(═O)NR⁶R⁷, —N(R⁷)C(═O)NR⁶R⁷, —NH₂, —NR⁶R⁷, —C(═O)N(H)R⁶, —C(═O)NR⁶R⁷;
   and
   which is optionally substituted, one or more times, identically or differently, with a C₁-C₆-alkyl-group;
R² represents a hydrogen atom or a group selected from phenyl-, pyridyl-;
   said group being substituted, one or more times, identically or differently, with a substituent selected from:
   halo-, hydroxy-, cyano-, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-,
   R⁹—, R⁹—(C₁-C₆-alkyl)-, R⁹—(CH₂)ₙ(CHOH)(CH₂)ₘ—, R⁹—(C₁-C₆-alkoxy)-, R⁹—(CH₂)ₙ(CHOH)(CH₂)ₚ—O—, R⁹—(C₁-C₆-alkoxy-C₁-C₆-alkyl)-, R⁹—(C₁-C₆-alkoxy-C₁-C₆-alkyl)-O—, —O—(CH₂)ₙ—C(═O)NR⁹R⁷, R⁹—O—, —C(═O)R⁹, —C(═O)O—R⁹, —OC(═O)—R⁹, —N(H)C(═O)R⁹, —N(R⁷)C(═O)R⁹, —N(H)C(═O)NR⁹R⁷, —N(R⁷)C(═O)NR⁹R⁷, —NR⁹R⁷, —C(═O)N(H)R⁹, —C(═O)NR⁹R⁷, R⁹—S—, R⁹—S(═O)—, R⁹—S(═O)₂—, —N(H)S(═O)R⁹, —N(R⁷)S(═O)R⁹, —S(═O)N(H)R⁹, —S(═O)NR⁹R⁷, —N(H)S(═O)₂R⁹, —N(R⁷)S(═O)₂R⁹, —S(═O)₂N(H)R⁹, —S(═O)₂NR⁹R⁷, —S(═O)(═NR⁹)R⁷, —S(═O)(═NR⁷)R⁹ or —N═S(═O)(R⁹)R⁷
or
R² represents a group selected from:

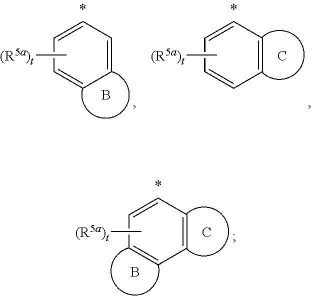

wherein * indicates the point of attachment of said group with the rest of the molecule;
B represents a 4- to 6-membered heterocyclic ring; which is optionally substituted, one or more times, identically or differently, with halo-,
   —CN, —OH, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R⁸—(C₁-C₆-alkoxy)-, R⁸—O—, —NR⁸R⁷, R⁸—S—, R⁸—S(═O)—, R⁸—S(═O)₂—, (C₃-C₆-cycloalkyl)-(CH₂)ₙ—O—;
C represents a 4- to 6-membered heterocyclic ring; which is optionally substituted, one or more times, identically or differently, with halo-,
   —CN, —OH, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R⁸—(C₁-C₆-alkoxy)-, R⁸—O—, —NR⁸R⁷, R⁸—S—, R⁸—S(═O)—, R⁸—S(═O)₂—, (C₃-C₆-cycloalkyl)-(CH₂)ₙ—O—;
each R^{5a}
   independently represents a group selected from:
   halo-, cyano-, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R⁸—(C₁-C₆-alkoxy)-, R⁸—O—, —NR⁸R⁷, R⁸—S—, R⁸—S(═O)—, R⁸—S(═O)₂—, (C₃-C₆-cycloalkyl)-(CH₂)ₙ—O—;
R⁶ represents a group selected from:
   C₁-C₆-alkyl-, C₃-C₆-cycloalkyl-, 3- to 10-membered heterocycloalkyl-, aryl-, heteroaryl-, —(CH₂)_{q}—(C₃-C₆-cycloalkyl), —(CH₂)_{q}-heteroaryl, —(CH₂)_{q}-(3- to 10-membered heterocycloalkyl), —(CH₂)_{q}-aryl;
   said group being optionally substituted, one or more times, identically or differently, with a substituent selected from:
   halo-, hydroxy-, cyano-, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R⁸—(C₁-C₆-alkyl)-, R⁸—(CH₂)ₙ(CHOH)(CH₂)ₘ—, R⁸—(C₁-C₆-alkoxy)-, R⁸—(CH₂)ₙ(CHOH)(CH₂)ₚ—O—, R⁸—(C₁-C₆-alkoxy-C₁-C₆-alkyl)-, R⁸—(C₁-C₆-alkoxy-C₁-C₆-alkyl)-O—, aryl-, R⁸—O—, —C(═O)R⁸, —C(═O)O—R⁸, —OC(═O)—R⁸, —N(H)C(═O)R⁸, —N(R⁷)C(═O)R⁸, —N(H)C(═O)NR⁸R⁷, —N(R⁷)C(═O)NR⁸R⁷, —NR⁸R⁷, —C(═O)N(H)R⁸, —C(═O)NR⁸R⁷, R⁸—S—, R⁸—S(═O)—, R⁸—S(═O)₂—, —N(H)S(═O)R⁸, —N(R⁷)S(═O)R⁸, —S(═O)N(H)R⁸, —S(═O)NR⁸R⁷, —N(H)S(═O)₂R⁸, —N(R⁷)S(═O)₂R⁸, —S(═O)₂N(H)R⁸, —S(═O)₂NR⁸R⁷, —S(═O)(═NR⁸)R⁷, —S(═O)(═NR⁷)R⁸, —N═S(═O)(R⁸)R⁷;
R⁷ represents a hydrogen atom, a C₁-C₆-alkyl-, or C₃-C₆-cycloalkyl-group;
or
R⁶ and R⁷,
   together with the nitrogen atom to which they are attached, represent a 3- to 10-membered heterocycloalkyl-group;
R⁸ represents a hydrogen atom, a C₁-C₆-alkyl- or C₃-C₆-cycloalkyl-group;
R⁹ represents a C₁-C₆-alkyl- or C₃-C₆-cycloalkyl-group;
or
R⁹ and R⁷,
   together with the nitrogen atom to which they are attached, represent a 3- to 10-membered heterocycloalkyl-group;
   which is optionally substituted with a halogen atom;
n, m, p
   represent, independently from each other, an integer of 0, 1, 2, 3, 4, or 5;
q represents an integer of 0, 1, 2 or 3;
and
t represents an integer of 0, 1 or 2;
or a stereoisomer, a tautomer, an N-oxide, a hydrate, a solvate, or a salt thereof, or a mixture of same.

The present invention further relates to methods of preparing compounds of general formula (I), to pharmaceutical compositions and combinations comprising said compounds, to the use of said compounds for manufacturing a pharmaceutical composition for the treatment or prophylaxis of a disease, as well as to intermediate compounds useful in the preparation of said compounds.

### DETAILED DESCRIPTION OF THE INVENTION

The terms as mentioned in the present text have preferably the following meanings:

The term “halogen atom”, “halo-” or “Hal-” is to be understood as meaning a fluorine, chlorine, bromine or iodine atom, preferably a fluorine, chlorine or bromine atom.

The term “C₁-C₁₀-alkyl” is to be understood as preferably meaning a linear or branched, saturated, monovalent hydrocarbon group having 1, 2, 3, 4, 5, 6, 7, 8, 9 or 10 carbon atoms, e.g. a methyl, ethyl, propyl, butyl, pentyl, hexyl, iso-propyl, iso-butyl, sec-butyl, tert-butyl, iso-pentyl, 2-methylbutyl, 1-methylbutyl, 1-ethylpropyl, 1,2-dimethylpropyl, neo-pentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethylbutyl, 1,1-dimethylbutyl, 2,3-dimethylbutyl, 1,3-dimethylbutyl, or 1,2-dimethylbutyl group, or an isomer thereof. Particularly, said group has 1, 2, 3, 4, 5 or 6 carbon atoms (“C₁-C₆-alkyl”), more particularly, said group has 1, 2, 3 or 4 carbon atoms (“C₁-C₄-alkyl”), e.g. a methyl, ethyl, propyl, butyl, iso-propyl, iso-butyl, sec-butyl, tert-butyl group; even more particularly 1, 2 or 3 carbon atoms (“C₁-C₃-alkyl”), e.g. a methyl, ethyl, n-propyl- or iso-propyl group.

The term “C₁-C₁₀-alkylene” is to be understood as preferably meaning a linear or branched, saturated, bivalent hydrocarbon group having 1, 2, 3, 4, 5, 6, 7, 8, 9 or 10 carbon atoms, e.g. a methylene, ethylene, n-propylene, n-butylene, n-pentylene, 2-methylbutylene, n-hexylene, 3-methylpentalene group, or an isomer thereof. Particularly, said group is linear and has 2, 3, 4 or 5 carbon atoms (“C₂-C₅-alkylene”), e.g. an ethylene, n-propylene, n-butylene, n-pentylene group, more particularly 3 or 4 carbon atoms (“C₃-C₄-alkylene”), e.g. an n-propylene or n-butylene group.

The term “halo-C₁-C₆-alkyl” is to be understood as preferably meaning a linear or branched, saturated, monovalent hydrocarbon group in which the term “C₁-C₆-alkyl” is defined supra, and in which one or more hydrogen atoms is replaced by a halogen atom, in identically or differently, i.e. one halogen atom being independent from another. Particularly, said halogen atom is F. Said halo-C₁-C₆-alkyl group is, for example, —CF₃, —CHF₂, —CH₂F, —CF₂CF₃, or —CH₂CF₃.

The term “hydroxy-C₁-C₆-alkyl-” is to be understood as preferably meaning a linear or branched, saturated, monovalent hydrocarbon group in which the term “C₁-C₆-alkyl-” is defined supra, and in which one or more of the hydrogen atoms is replaced by a hydroxy group with the proviso that not more than one hydrogen atom attached to a single carbon atom is being replaced. Said hydroxy-C₁-C₆-alkyl-group is, for example, —CH₂OH, —CH₂CH₂—OH, —C(OH)H—CH₃, or —C(OH)H—CH₂OH.

The term “C₁-C₆-alkoxy” is to be understood as preferably meaning a linear or branched, saturated, monovalent, hydrocarbon group of formula —O—(C₁-C₆-alkyl), in which the term “C₁-C₆-alkyl” is defined supra, e.g. a methoxy, ethoxy, n-propoxy, iso-propoxy, n-butoxy, iso-butoxy, tert-butoxy, sec-butoxy, pentoxy, iso-pentoxy, or n-hexoxy group, or an isomer thereof.

The term “halo-C₁-C₆-alkoxy” is to be understood as preferably meaning a linear or branched, saturated, monovalent C₁-C₆-alkoxy group, as defined supra, in which one or more of the hydrogen atoms is replaced, in identically or differently, by a halogen atom. Particularly, said halogen atom is F. Said halo-C₁-C₆-alkoxy group is, for example, —OCF₃, —OCHF₂, —OCH₂F, —OCF₂CF₃, or —OCH₂CF₃.

The term “C₁-C₆-alkoxy-C₁-C₆-alkyl” is to be understood as preferably meaning a linear or branched, saturated, monovalent C₁-C₆-alkyl group, as defined supra, in which one or more of the hydrogen atoms is replaced, in identically or differently, by a C₁-C₆-alkoxy group, as defined supra, e.g. methoxyalkyl, ethoxyalkyl, propyloxyalkyl, iso-propoxyalkyl, butoxyalkyl, iso-butoxyalkyl, tert-butoxyalkyl, sec-butoxyalkyl, pentyloxyalkyl, iso-pentyloxyalkyl, hexyloxyalkyl group, or an isomer thereof.

The term “halo-C₁-C₆-alkoxy-C₁-C₆-alkyl” is to be understood as preferably meaning a linear or branched, saturated, monovalent C₁-C₆-alkoxy-C₁-C₆-alkyl group, as defined supra, in which one or more of the hydrogen atoms is replaced, in identically or differently, by a halogen atom. Particularly, said halogen atom is F. Said halo-C₁-C₆-alkoxy-C₁-C₆-alkyl group is, for example, —CH₂CH₂OCF₃, —CH₂CH₂OCHF₂, —CH₂CH₂OCH₂F, —CH₂CH₂OCF₂CF₃, or —CH₂CH₂OCH₂CF₃.

The term “C₂-C₁₀-alkenyl” is to be understood as preferably meaning a linear or branched, monovalent hydrocarbon group, which contains one or more double bonds, and which has 2, 3, 4, 5, 6, 7, 8, 9 or 10 carbon atoms, particularly 2, 3, 4, 5 or 6 carbon atoms (“C₂-C₆-alkenyl”), more particularly 2 or 3 carbon atoms (“C₂-C₃-alkenyl”), it being understood that in the case in which said alkenyl group contains more than one double bond, then said double bonds may be isolated from, or conjugated with, each other. Said alkenyl group is, for example, a vinyl, allyl, (E)-2-methylvinyl, (Z)-2-methylvinyl, homoallyl, (E)-but-2-enyl, (Z)-but-2-enyl, (E)-but-1-enyl, (Z)-but-1-enyl, pent-4-enyl, (E)-pent-3-enyl, (Z)-pent-3-enyl, (E)-pent-2-enyl, (Z)-pent-2-enyl, (E)-pent-1-enyl, (Z)-pent-1-enyl, hex-5-enyl, (E)-hex-4-enyl, (Z)-hex-4-enyl, (E)-hex-3-enyl, (Z)-hex-3-enyl, (E)-hex-2-enyl, (Z)-hex-2-enyl, (E)-hex-1-enyl, (Z)-hex-1-enyl, iso-propenyl, 2-methylprop-2-enyl, 1-methylprop-2-enyl, 2-methylprop-1-enyl, (E)-1-methylprop-1-enyl, (Z)-1-methylprop-1-enyl, 3-methylbut-3-enyl, 2-methylbut-3-enyl, 1-methylbut-3-enyl, 3-methylbut-2-enyl, (E)-2-methylbut-2-enyl, (Z)-2-methylbut-2-enyl, (E)-1-methylbut-2-enyl, (Z)-1-methylbut-2-enyl, (E)-3-methylbut-1-enyl, (Z)-3-methylbut-1-enyl, (E)-2-methylbut-1-enyl, (Z)-2-methylbut-1-enyl, (E)-1-methylbut-1-enyl, (Z)-1-methylbut-1-enyl, 1,1-dimethylprop-2-enyl, 1-ethylprop-1-enyl, 1-propylvinyl, 1-isopropylvinyl, 4-methylpent-4-enyl, 3-methylpent-4-enyl, 2-methylpent-4-enyl, 1-methylpent-4-enyl, 4-methylpent-3-enyl, (E)-3-methylpent-3-enyl, (Z)-3-methylpent-3-enyl, (E)-2-methylpent-3-enyl, (Z)-2-methylpent-3-enyl, (E)-1-methylpent-3-enyl, (Z)-1-methylpent-3-enyl, (E)-4-methylpent-2-enyl, (Z)-4-methylpent-2-enyl, (E)-3-methylpent-2-enyl, (Z)-3-methylpent-2-enyl, (E)-2-methylpent-2-enyl, (Z)-2-methylpent-2-enyl, (E)-1-methylpent-2-enyl, (Z)-1-methylpent-2-enyl, (E)-4-methylpent-1-enyl, (Z)-4-methylpent-1-enyl, (E)-3-methylpent-1-enyl, (Z)-3-methylpent-1-enyl, (E)-2-methylpent-1-enyl, (Z)-2-methylpent-1-enyl, (E)-1-methylpent-1-enyl, (Z)-1-methylpent-1-enyl, 3-ethylbut-3-enyl, 2-ethylbut-3-enyl, 1-ethylbut-3-enyl, (E)-3-ethylbut-2-enyl, (Z)-3-ethylbut-2-enyl, (E)-2-ethylbut-2-enyl, (Z)-2-ethylbut-2-enyl, (E)-1-ethylbut-2-enyl, (Z)-1-ethylbut-2-enyl, (E)-3-ethylbut-1-enyl, (Z)-3-ethylbut-1-enyl, 2-ethylbut-1-enyl, (E)-1-ethylbut-1-enyl, (Z)-1-ethylbut-1-enyl, 2-propylprop-2-enyl, 1-propylprop-2-enyl, 2-isopropylprop-2-enyl, 1-isopropylprop-2-enyl, (E)-2-propylprop-1-enyl, (Z)-2-propylprop-1-enyl, (E)-1-propylprop-1-enyl, (Z)-1-propylprop-1-enyl, (E)-2-isopropylprop-1-enyl, (Z)-2-isopropylprop-1-enyl, (E)-1-isopropylprop-1-enyl, (Z)-1-isopropylprop-1-enyl, (E)-3,3-dimethylprop-1-enyl, (Z)-3,3-dimethylprop-1-enyl, 1-(1,1-dimethylethyl)ethenyl, buta-1,3-dienyl, penta-1,4-dienyl, hexa-1,5-dienyl, or methylhexadienyl group. Particularly, said group is vinyl or allyl.

The term “C₂-C₁₀-alkynyl” is to be understood as preferably meaning a linear or branched, monovalent hydrocarbon group which contains one or more triple bonds, and which contains 2, 3, 4, 5, 6, 7, 8, 9 or 10 carbon atoms, particularly 2, 3, 4, 5 or 6 carbon atoms (“C₂-C₆-alkynyl”), more particularly 2 or 3 carbon atoms (“C₂-C₃-alkynyl”). Said C₂-C₁₀-alkynyl group is, for example, ethynyl, prop-1-ynyl, prop-2-ynyl, but-1-ynyl, but-2-ynyl, but-3-ynyl, pent-1-ynyl, pent-2-ynyl, pent-3-ynyl, pent-4-ynyl, hex-1-ynyl, hex-2-ynyl, hex-3-ynyl, hex-4-ynyl, hex-5-ynyl, 1-methylprop-2-ynyl, 2-methylbut-3-ynyl, 1-methylbut-3-ynyl, 1-methylbut-2-ynyl, 3-methylbut-1-ynyl, 1-ethylprop-2-ynyl, 3-methylpent-4-ynyl, 2-methylpent-4-ynyl, 1-methylpent-4-ynyl, 2-methylpent-3-ynyl, 1-methylpent-3-ynyl, 4-methylpent-2-ynyl, 1-methylpent-2-ynyl, 4-methylpent-1-ynyl, 3-methylpent-1-ynyl, 2-ethylbut-3-ynyl, 1-ethylbut-3-ynyl, 1-ethylbut-2-ynyl, 1-propylprop-2-ynyl, 1-isopropylprop-2-ynyl, 2,2-dimethylbut-3-ynyl, 1,1-dimethylbut-3-ynyl, 1,1-dimethylbut-2-ynyl, or 3,3-dimethylbut-1-ynyl group. Particularly, said alkynyl group is ethynyl, prop-1-ynyl, or prop-2-ynyl.

The term “C₃-C₁₀-cycloalkyl” is to be understood as meaning a saturated, monovalent, mono-, or bicyclic hydrocarbon ring which contains 3, 4, 5, 6, 7, 8, 9 or 10 carbon atoms (“C₃-C₁₀-cycloalkyl”). Said C₃-C₁₀-cycloalkyl group is for example, a monocyclic hydrocarbon ring, e.g. a cyclopropyl, cyclobutyl, cyclopentyl, cyclohexyl, cycloheptyl, cyclooctyl, cyclononyl or cyclodecyl, or a bicyclic hydrocarbon ring, e.g. a perhydropentalenylene or decalin ring. Particularly, said ring contains 3, 4, 5 or 6 carbon atoms (“C₃-C₆-cycloalkyl”).

The term “C₃-C₆-cycloalkyloxy” refers to a (C₃-C₆-cycloalkyl)-O— group in which “C₃-C₆-cycloalkyl” is as defined herein. Examples include, but are not limited to, cyclopropanoxy and cyclobutanoxy.

The term “C₄-C₁₀-cycloalkenyl” is to be understood as preferably meaning a non-aromatic, monovalent, mono-, or bicyclic hydrocarbon ring which contains 4, 5, 6, 7, 8, 9 or 10 carbon atoms and one, two, three or four double bonds, in conjugation or not, as the size of said cycloalkenyl ring allows. Said C₄-C₁₀-cycloalkenyl group is for example, a monocyclic hydrocarbon ring, e.g. a cyclobutenyl, cyclopentenyl, or cyclohexenyl or a bicyclic hydrocarbon, e.g.

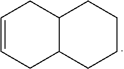

The term “C₅-C₈-cycloalkenyloxy” refers to a (C₅-C₈-cycloalkenyl)-O— group in which “C₅-C₈-cycloalkenyl” is as defined herein.

The term “3- to 10-membered heterocycloalkyl”, is to be understood as meaning a saturated, monovalent, mono- or bicyclic hydrocarbon ring which contains 2, 3, 4, 5, 6, 7, 8 or 9 carbon atoms, and one or more heteroatom-containing groups selected from: —C(═O)—, —O—, —S—, —S(═O)—, —S(═O)₂—, —N(R^{a})—, in which R^{a} represents a hydrogen atom or a C₁-C₆-alkyl-group; it being possible for said heterocycloalkyl group to be attached to the rest of the molecule via any one of the carbon atoms or, if present, the nitrogen atom.

Particularly, said 3- to 10-membered heterocycloalkyl can contain 2, 3, 4, or 5 carbon atoms, and one or more of the above-mentioned heteroatom-containing groups (a “3- to 6-membered heterocycloalkyl”), more particularly said heterocycloalkyl can contain 4 or 5 carbon atoms, and one or more of the above-mentioned heteroatom-containing groups (a “5- to 6-membered heterocycloalkyl”).

Particularly, without being limited thereto, said heterocycloalkyl can be a 4-membered ring, such as an azetidinyl, oxetanyl, or a 5-membered ring, such as tetrahydrofuranyl, dioxolinyl, pyrrolidinyl, imidazolidinyl, pyrazolidinyl, or a 6-membered ring, such as tetrahydropyranyl, piperidinyl, morpholinyl, dithianyl, thiomorpholinyl, piperazinyl, or trithianyl, or a 7-membered ring, such as a diazepanyl ring, for example.

Said heterocycloalkyl can be bicyclic, such as, without being limited thereto, a 5,5-membered ring, e.g. a hexahydrocyclopenta[c]pyrrol-2(1H)-yl ring, or a 5,6-membered bicyclic ring, e.g. a hexahydropyrrolo[1,2-a]pyrazin-2(1H)-yl ring.

Said heterocycloalkyl can be spirocyclic, such as, without being limited thereto, e.g. a 2-oxa-6-azaspiro[3.3]heptane ring or a 2-oxa-6-azaspiro[3.4]octane ring or a 2-oxa-7-azaspiro[4.4]nonane ring.

The term “4- to 10-membered heterocycloalkenyl”, is to be understood as meaning an non-aromatic, unsaturated, monovalent, mono- or bicyclic hydrocarbon ring which contains 3, 4, 5, 6, 7, 8 or 9 carbon atoms, and one or more heteroatom-containing groups selected from:

—C(═O)—, —O—, —S—, —S(═O)—, —S(═O)₂—, —N(R^{a})—, in which R^{a} represents a hydrogen atom or a C₁-C₆-alkyl-group; it being possible for said heterocycloalkenyl group to be attached to the rest of the molecule via any one of the carbon atoms or, if present, the nitrogen atom. Examples of said heterocycloalkenyl are e.g. 4H-pyranyl, 2H-pyranyl, 3H-diazirinyl, 2,5-dihydro-1H-pyrrolyl, [1,3]dioxolyl, 4H-[1,3,4]thiadiazinyl, 2,5-dihydrofuranyl, 2,3-dihydrofuranyl, 2,5-dihydrothiophenyl, 2,3-dihydrothiophenyl, 4,5-dihydrooxazolyl, or 4H-[1,4]thiazinyl group.

The term “heterocyclic ring”, as used in the term “4-, 5- or 6-membered heterocyclic ring”, or “4- to 6-membered heterocyclic ring” or “4- to 5-membered heterocyclic ring”, for example, as used in the definition of compounds of general formula (I) as defined herein, is to be understood as meaning a saturated, partially unsaturated or aromatic monocyclic hydrocarbon ring which contains 1, 2, 3, 4 or 5 carbon atoms, and one or more heteroatom-containing groups selected from —C(═O)—, —O—, —S—, —S(═O)—, —S(═O)₂—, ═N—, —N(H)—, —N(R″)—, wherein R″ represents a C₁-C₆-alkyl, C₃-C₆-cycloalkyl, —C(═O)—(C₁-C₆-alkyl) or —C(═O)—(C₁-C₆-cycloalkyl) group.

The term “aryl” is to be understood as preferably meaning a monovalent, aromatic or partially aromatic, mono-, or bi- or tricyclic hydrocarbon ring having 6, 7, 8, 9, 10, 11, 12, 13 or 14 carbon atoms (a “C₆-C₁₄-aryl” group), particularly a ring having 6 carbon atoms (a “C₆-aryl” group), e.g. a phenyl group; or a biphenyl group, or a ring having 9 carbon atoms (a “C₉-aryl” group), e.g. an indanyl or indenyl group, or a ring having 10 carbon atoms (a “C₁₀-aryl” group), e.g. a tetralinyl, dihydronaphthyl, or naphthyl group, or a ring having 13 carbon atoms, (a “C₁₃-aryl” group), e.g. a fluorenyl group, or a ring having 14 carbon atoms, (a “C₁₄-aryl” group), e.g. an anthranyl group. Preferably, the aryl group is a phenyl group.

The term “heteroaryl” is understood as preferably meaning a monovalent, monocyclic, bicyclic or tricyclic aromatic ring system having 5, 6, 7, 8, 9, 10, 11, 12, 13 or 14 ring atoms (a “5- to 14-membered heteroaryl” group), particularly 5 or 6 or 9 or 10 atoms, and which contains at least one heteroatom which may be identical or different, said heteroatom being such as oxygen, nitrogen or sulfur, and in addition in each case can be benzocondensed. Particularly, heteroaryl is selected from thienyl, furanyl, pyrrolyl, oxazolyl, thiazolyl, imidazolyl, pyrazolyl, isoxazolyl, isothiazolyl, oxadiazolyl, triazolyl, thiadiazolyl, thia-4H-pyrazolyl etc., and benzo derivatives thereof, such as, for example, benzofuranyl, benzothienyl, benzoxazolyl, benzisoxazolyl, benzimidazolyl, benzotriazolyl, indazolyl, indolyl, isoindolyl, etc.; or pyridyl, pyridazinyl, pyrimidinyl, pyrazinyl, triazinyl, etc., and benzo derivatives thereof, such as, for example, quinolinyl, quinazolinyl, isoquinolinyl, etc.; or azocinyl, indolizinyl, purinyl, etc., and benzo derivatives thereof; or cinnolinyl, phthalazinyl, quinazolinyl, quinoxalinyl, naphthpyridinyl, pteridinyl, carbazolyl, acridinyl, phenazinyl, phenothiazinyl, phenoxazinyl, xanthenyl, or oxepinyl, etc.

The term “C₁-C₆”, as used throughout this text, e.g. in the context of the definition of “C₁-C₆-alkyl”, “C₁-C₆-haloalkyl”, “C₁-C₆-alkoxy”, or “C₁-C₆-haloalkoxy” is to be understood as meaning an alkyl group having a finite number of carbon atoms of 1 to 6, i.e. 1, 2, 3, 4, 5, or 6 carbon atoms. It is to be understood further that said term “C₁-C₆” is to be interpreted as any sub-range comprised therein, e.g. C₁-C₆, C₂-C₅, C₃-C₄, C₁-C₂, C₁-C₃, C₁-C₄, C₁-C₅; particularly C₁-C₂, C₁-C₃, C₁-C₄, C₁-C₅, C₁-C₆; more particularly C₁-C₄; in the case of “C₁-C₆-haloalkyl” or “C₁-C₆-haloalkoxy” even more particularly C₁-C₂.

Similarly, as used herein, the term “C₂-C₆”, as used throughout this text, e.g. in the context of the definitions of “C₂-C₆-alkenyl” and “C₂-C₆-alkynyl”, is to be understood as meaning an alkenyl group or an alkynyl group having a finite number of carbon atoms of 2 to 6, i.e. 2, 3, 4, 5, or 6 carbon atoms. It is to be understood further that said term “C₂-C₆” is to be interpreted as any sub-range comprised therein, e.g. C₂-C₆, C₃-C₅, C₃-C₄, C₂-C₃, C₂-C₄, C₂-C₅; particularly C₂-C₃.

Further, as used herein, the term “C₃-C₆”, as used throughout this text, e.g. in the context of the definition of “C₃-C₆-cycloalkyl”, is to be understood as meaning a cycloalkyl group having a finite number of carbon atoms of 3 to 6, i.e. 3, 4, 5 or 6 carbon atoms. It is to be understood further that said term “C₃-C₆” is to be interpreted as any sub-range comprised therein, e.g. C₃-C₆, C₄-C₅, C₃-C₅, C₃-C₄, C₄-C₆, C₅-C₆; particularly C₃-C₆.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds.

The term “optionally substituted” means optional substitution with the specified groups, radicals or moieties.

As used herein, the term “Leaving group” refers to an atom or a group of atoms that is displaced in a chemical reaction as stable species taking with it the bonding electrons. Preferably, a leaving group is selected from the group comprising: halo, in particular chloro, bromo or iodo, methanesulfonyloxy, p-toluenesulfonyloxy, trifluoromethanesulfonyloxy, nonafluorobutanesulfonyloxy, (4-bromo-benzene)sulfonyloxy, (4-nitro-benzene)sulfonyloxy, (2-nitro-benzene)-sulfonyloxy, (4-isopropyl-benzene)sulfonyloxy, (2,4,6-tri-isopropyl-benzene)-sulfonyloxy, (2,4,6-trimethyl-benzene)sulfonyloxy, (4-tertbutyl-benzene)sulfonyloxy, benzenesulfonyloxy, and (4-methoxy-benzene)sulfonyloxy.

As used herein, the term “protective group” is a protective group attached to a nitrogen in intermediates used for the preparation of compounds of the general formula I. Such groups are introduced e.g. by chemical modification of the respective amino group in order to obtain chemoselectivity in a subsequent chemical reaction. Protective groups for amino groups are described for example in T. W. Greene and P. G. M. Wuts in Protective Groups in Organic Synthesis, 3^{rd} edition, Wiley 1999; more specifically, said groups can be selected from substituted sulfonyl groups, such as mesyl-, tosyl- or phenylsulfonyl-, acyl groups such as benzoyl, acetyl or tetrahydropyranyl-, or carbamate based groups, such as tert.-butoxycarbonyl (Boc), or can include silicon, as in e.g. 2-(trimethylsilyl)ethoxymethyl (SEM).

As used herein, the term “one or more”, e.g. in the definition of the substituents of the compounds of the general formulae of the present invention, is understood as meaning “one, two, three, four or five, particularly one, two, three or four, more particularly one, two or three, even more particularly one or two”.

The invention also includes all suitable isotopic variations of a compound of the invention. An isotopic variation of a compound of the invention is defined as one in which at least one atom is replaced by an atom having the same atomic number but an atomic mass different from the atomic mass usually or predominantly found in nature. Examples of isotopes that can be incorporated into a compound of the invention include isotopes of hydrogen, carbon, nitrogen, oxygen, phosphorus, sulphur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I, respectively. Certain isotopic variations of a compound of the invention, for example, those in which one or more radioactive isotopes such as ³H or ¹⁴C are incorporated, are useful in drug and/or substrate tissue distribution studies. Tritiated and carbon-14, i.e., ¹⁴C, isotopes are particularly preferred for their ease of preparation and detectability. Further, substitution with isotopes such as deuterium may afford certain therapeutic advantages resulting from greater metabolic stability, for example, increased in vivo half-life or reduced dosage requirements and hence may be preferred in some circumstances. Isotopic variations of a compound of the invention can generally be prepared by conventional procedures known by a person skilled in the art such as by the illustrative methods or by the preparations described in the examples hereafter using appropriate isotopic variations of suitable reagents.

Where the plural form of the word compounds, salts, polymorphs, hydrates, solvates and the like, is used herein, this is taken to mean also a single compound, salt, polymorph, isomer, hydrate, solvate or the like.

By “stable compound” or “stable structure” is meant a compound that is sufficiently robust to survive isolation to a useful degree of purity from a reaction mixture, and formulation into an efficacious therapeutic agent.

The compounds of this invention may contain one or more asymmetric centre, depending upon the location and nature of the various substituents desired. Asymmetric carbon atoms may be present in the (R) or (S) configuration, resulting in racemic mixtures in the case of a single asymmetric centre, and diastereomeric mixtures in the case of multiple asymmetric centres. In certain instances, asymmetry may also be present due to restricted rotation about a given bond, for example, the central bond adjoining two substituted aromatic rings of the specified compounds.

The compounds of the present invention may contain sulphur atoms which are asymmetric, such as an asymmetric sulphoxide or sulphoximine group, of structure:

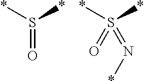

for example, in which * indicates atoms to which the rest of the molecule can be bound.

Substituents on a ring may also be present in either cis or trans form. It is intended that all such configurations (including enantiomers and diastereomers), are included within the scope of the present invention.

Preferred compounds are those which produce the more desirable biological activity. Separated, pure or partially purified isomers and stereoisomers or racemic or diastereomeric mixtures of the compounds of this invention are also included within the scope of the present invention. The purification and the separation of such materials can be accomplished by standard techniques known in the art.

Pure stereoisomers can be obtained by resolution of racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., chiral HPLC columns), with or without conventional derivatisation, optimally chosen to maximise the separation of the enantiomers. Suitable chiral HPLC columns are manufactured by Daicel, e.g., Chiracel OD and Chiracel OJ among many others, all routinely selectable. Enzymatic separations, with or without derivatisation, are also useful. The optically active compounds of this invention can likewise be obtained by chiral syntheses utilizing optically active starting materials.

In order to limit different types of isomers from each other reference is made to IUPAC Rules Section E (Pure Appl Chem 45, 11-30, 1976).

The present invention includes all possible stereoisomers of the compounds of the present invention as single stereoisomers, or as any mixture of said stereoisomers, e.g. (R) or (S) isomers, or (E) or (Z) isomers, in any ratio. Isolation of a single stereoisomer, e.g. a single enantiomer or a single diastereomer, of a compound of the present invention may be achieved by any suitable state of the art method, such as chromatography, especially chiral chromatography, for example.

Further, the compounds of the present invention may exist as tautomers. For example, any compound of the present invention which contains a pyrazole moiety as a heteroaryl group for example can exist as a 1H tautomer, or a 2H tautomer, or even a mixture in any amount of the two tautomers, or a triazole moiety for example can exist as a 1H tautomer, a 2H tautomer, or a 4H tautomer, or even a mixture in any amount of said 1H, 2H and 4H tautomers, namely:

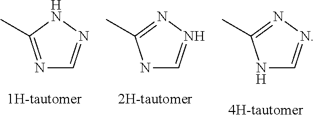

The present invention includes all possible tautomers of the compounds of the present invention as single tautomers, or as any mixture of said tautomers, in any ratio.

Further, the compounds of the present invention can exist as N-oxides, which are defined in that at least one nitrogen of the compounds of the present invention is oxidised. The present invention includes all such possible N-oxides.

The present invention also relates to useful forms of the compounds as disclosed herein, such as metabolites, hydrates, solvates, salts, in particular pharmaceutically acceptable salts, and co-precipitates.

The compounds of the present invention can exist as a hydrate, or as a solvate, wherein the compounds of the present invention contain polar solvents, in particular water, methanol or ethanol for example as structural element of the crystal lattice of the compounds. The amount of polar solvents, in particular water, may exist in a stoichiometric or non-stoichiometric ratio. In the case of stoichiometric solvates, e.g. a hydrate, hemi-, (semi-), mono-, sesqui-, di-, tri-, tetra-, penta-etc. solvates or hydrates, respectively, are possible. The present invention includes all such hydrates or solvates.

Further, the compounds of the present invention can exist in free form, e.g. as a free base, or as a free acid, or as a zwitterion, or can exist in the form of a salt. Said salt may be any salt, either an organic or inorganic addition salt, particularly any pharmaceutically acceptable organic or inorganic addition salt, customarily used in pharmacy.

The term “pharmaceutically acceptable salt” refers to a relatively non-toxic, inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, such as hydrochloric, hydrobromic, hydroiodic, sulfuric, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, persulfuric, 3-phenylpropionic, picric, pivalic, 2-hydroxyethanesulfonate, itaconic, sulfamic, trifluoromethanesulfonic, dodecylsulfuric, ethansulfonic, benzenesulfonic, para-toluenesulfonic, methansulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, hemisulfuric, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium or magnesium salt, an ammonium salt or a salt with an organic base which affords a physiologically acceptable cation, for example a salt with N-methyl-glucamine, dimethyl-glucamine, ethyl-glucamine, tysine, dicyclohexylamine, 1,6-hexadiamine, ethanolamine, glucosamine, sarcosine, serinol, tris-hydroxy-methyl-aminomethane, aminopropandiol, sovak-base, 1-amino-2,3,4-butantriol, or with a quaternary ammonium salt, such as tetramethylammonium, tetraethylammonium, tetra(n-propyl)ammonium, tetra (n-butyl)ammonium, or N-benzyl-N,N,N-trimethylammonium.

Those skilled in the art will further recognise that acid addition salts of the claimed compounds may be prepared by reaction of the compounds with the appropriate inorganic or organic acid via any of a number of known methods. Alternatively, alkali and alkaline earth metal salts of acidic compounds of the invention are prepared by reacting the compounds of the invention with the appropriate base via a variety of known methods.

The present invention includes all possible salts of the compounds of the present invention as single salts, or as any mixture of said salts, in any ratio.

Furthermore, the present invention includes all possible crystalline forms, or polymorphs, of the compounds of the present invention, either as single polymorphs, or as a mixture of more than one polymorphs, in any ratio.

In accordance with a first aspect, the present invention covers compounds of general formula (I):

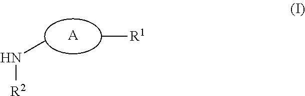

in which:
A is selected from:

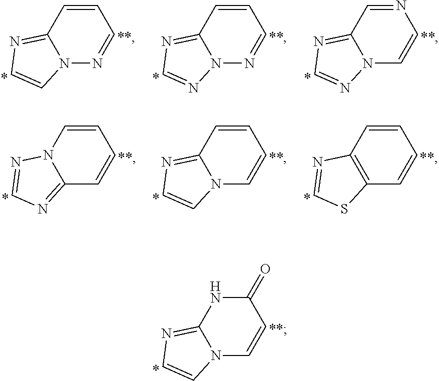

wherein * represents the point of attachment to the nitrogen atom and ** represents the point of attachment to the R¹ group;
R¹ represents a phenyl-group
   which is substituted, one or more times, identically or differently, with a substituent selected from:
   —OH, —N(H)C(═O)R⁶, —N(R⁷)C(═O)R⁶, —N(H)C(═O)NR⁶R⁷, —N(R⁷)C(═O)NR⁶R⁷, —NH₂, —NR⁶R⁷, —C(═O)N(H)R⁶, —C(═O)NR⁶R⁷;
   and
   which is optionally substituted, one or more times, identically or differently, with a C₁-C₆-alkyl-group;
R² represents a hydrogen atom or a group selected from phenyl-, pyridyl-;
   said group being substituted, one or more times, identically or differently, with a substituent selected from:
   halo-, hydroxy-, cyano-, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-,
   R⁹—, R⁹—(C₁-C₆-alkyl)-, R⁹—(CH₂)ₙ(CHOH)(CH₂)ₘ—, R⁹—(C₁-C₆-alkoxy)-, R⁹—(CH₂)ₙ(CHOH)(CH₂)ₚ—O—, R⁹—(C₁-C₆-alkoxy-C₁-C₆-alkyl)-, R⁹—(C₁-C₆-alkoxy-C₁-C₆-alkyl)-O—, —O—(CH₂)ₙ—C(═O)NR⁹R⁷, R⁹—O—, —C(═O)R⁹, —C(═O)O—R⁹, —OC(═O)—R⁹, —N(H)C(═O)R⁹, —N(R⁷)C(═O)R⁹, —N(H)C(═O)NR⁹R⁷, —N(R⁷)C(═O)NR⁹R⁷, —NR⁹R⁷, —C(═O)N(H)R⁹, —C(═O)NR⁹R⁷, R⁹—S—, R⁹—S(═O)—, R⁹—S(═O)₂—, —N(H)S(═O)R⁹, —N(R⁷)S(═O)R⁹, —S(═O)N(H)R⁹, —S(═O)NR⁹R⁷, —N(H)S(═O)₂R⁹, —N(R⁷)S(═O)₂R⁹, —S(═O)₂N(H)R⁹, —S(═O)₂NR⁹R⁷, —S(═O)(═NR⁹)R⁷, —S(═O)(═NR⁷)R⁹ or —N═S(═O)(R⁹)R⁷;
or
R² represents a group selected from:

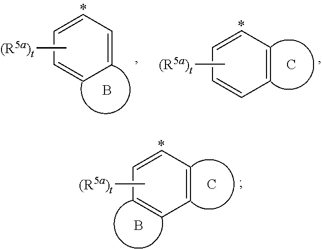

wherein * indicates the point of attachment of said group with the rest of the molecule;
B represents a 4- to 6-membered heterocyclic ring; which is optionally substituted, one or more times, identically or differently, with halo-,
   —CN, —OH, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R⁸—(C₁-C₆-alkoxy)-, R⁸—O—, —NR⁸R⁷, R⁸—S—, R⁸—S(═O)—, R⁸—S(═O)₂—, (C₃-C₆-cycloalkyl)-(CH₂)ₙ—O—;
C represents a 4- to 6-membered heterocyclic ring; which is optionally substituted, one or more times, identically or differently, with halo-,
   —CN, —OH, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R⁸—(C₁-C₆-alkoxy)-, R⁸—O—, —NR⁸R⁷, R⁸—S—, R⁸—S(═O)—, R⁸—S(═O)₂—, (C₃-C₆-cycloalkyl)-(CH₂)ₙ—O—;
each R^{5a}
   independently represents a group selected from:
   halo-, cyano-, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R⁸—(C₁-C₆-alkoxy)-, R⁸—O—, —NR⁸R⁷, R⁸—S—, R⁸—S(═O)—, R⁸—S(═O)₂—, (C₃-C₆-cycloalkyl)-(CH₂)ₙ—O—;
R⁶ represents a group selected from:
   C₁-C₆-alkyl-, C₃-C₆-cycloalkyl-, 3- to 10-membered heterocycloalkyl-, aryl-, heteroaryl-, —(CH₂)_{q}—(C₃-C₆-cycloalkyl), —(CH₂)_{q}-heteroaryl, —(CH₂)_{q}-(3- to 10-membered heterocycloalkyl), —(CH₂)_{q}-aryl;
   said group being optionally substituted, one or more times, identically or differently, with a substituent selected from:
   halo-, hydroxy-, cyano-, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R⁸—(C₁-C₆-alkyl)-, R⁸—(CH₂)ₙ(CHOH)(CH₂)ₘ—, R⁸—(C₁-C₆-alkoxy)-, R⁸—(CH₂)ₙ(CHOH)(CH₂)ₚ—O—, R⁸—(C₁-C₆-alkoxy-C₁-C₆-alkyl)-, R⁸—(C₁-C₆-alkoxy-C₁-C₆-alkyl)-O—, aryl-, R⁸—O—, —C(═O)R⁸, —C(═O)O—R⁸, —OC(═O)—R⁸, —N(H)C(═O)R⁸, —N(R⁷)C(═O)R⁸, —N(H)C(═O)NR⁸R⁷, —N(R⁷)C(═O)NR⁸R⁷, —NR⁸R⁷, —C(═O)N(H)R⁸, —C(═O)NR⁸R⁷, R⁸—S—, R⁸—S(═O)—, R⁸—S(═O)₂—, —N(H)S(═O)R⁸, —N(R⁷)S(═O)R⁸, —S(═O)N(H)R⁸, —S(═O)NR⁸R⁷, —N(H)S(═O)₂R⁸, —N(R⁷)S(═O)₂R⁸, —S(═O)₂N(H)R⁸, —S(═O)₂NR⁸R⁷, —S(═O)(═NR⁸)R⁷, —S(═O)(═NR⁷)R⁸, —N═S(═O)(R⁸)R⁷;
R⁷ represents a hydrogen atom, a C₁-C₆-alkyl-, or C₃-C₆-cycloalkyl-group;
or
R⁶ and R⁷,
   together with the nitrogen atom to which they are attached, represent a 3- to 10-membered heterocycloalkyl-group;
R⁸ represents a hydrogen atom, a C₁-C₆-alkyl- or C₃-C₆-cycloalkyl-group;
R⁹ represents a C₁-C₆-alkyl- or C₃-C₆-cycloalkyl-group;
or
R⁹ and R⁷,
   together with the nitrogen atom to which they are attached, represent a 3- to 10-membered heterocycloalkyl-group;
   which is optionally substituted with a halogen atom, preferably with fluoro;
n, m, p
   represent, independently from each other, an integer of 0, 1, 2, 3, 4, or 5;
q represents an integer of 0, 1, 2 or 3;
and
t represents an integer of 0, 1 or 2;
or a stereoisomer, a tautomer, an N-oxide, a hydrate, a solvate, or a salt thereof, or a mixture of same.

In a preferred embodiment, the invention relates to compounds of formula (I), wherein:
A represents:

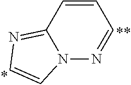

wherein * represents the point of attachment to the nitrogen atom and ** represents the point of attachment to the R¹ group.

In another preferred embodiment, the invention relates to compounds of formula (I), wherein:
A represents:

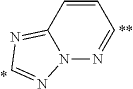

wherein * represents the point of attachment to the nitrogen atom and ** represents the point of attachment to the R¹ group.

In another preferred embodiment, the invention relates to compounds of formula (I), wherein:
A represents:

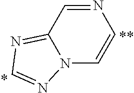

wherein * represents the point of attachment to the nitrogen atom and ** represents the point of attachment to the R¹ group.

In another preferred embodiment, the invention relates to compounds of formula (I), wherein:
A represents:

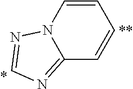

wherein * represents the point of attachment to the nitrogen atom and ** represents the point of attachment to the R¹ group.

In another preferred embodiment, the invention relates to compounds of formula (I), wherein:
A represents:

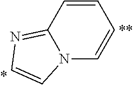

wherein * represents the point of attachment to the nitrogen atom and ** represents the point of attachment to the R¹ group.

In another preferred embodiment, the invention relates to compounds of formula (I), wherein:
A represents:

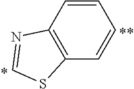

wherein * represents the point of attachment to the nitrogen atom and ** represents the point of attachment to the R¹ group.

In another preferred embodiment, the invention relates to compounds of formula (I), wherein:
A represents:

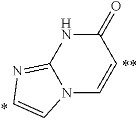

wherein * represents the point of attachment to the nitrogen atom and ** represents the point of attachment to the R¹ group.

In another preferred embodiment, the invention relates to compounds of formula (I), wherein:
R¹ represents a phenyl group
   which is substituted, one or more times, identically or differently, with a substituent selected from:
   —OH, —N(H)C(═O)R⁶, —NH₂, —C(═O)N(H)R⁶;
   and
   which is optionally substituted, one or more times, identically or differently, with a C₁-C₆-alkyl-group.

In another preferred embodiment, the invention relates to compounds of formula (I), wherein:
R¹ represents a phenyl group
   which is substituted, one or more times, identically or differently, with a substituent selected from:
   —N(H)C(═O)R⁶, —C(═O)N(H)R⁶.

In another preferred embodiment, the invention relates to compounds of formula (I), wherein:
R¹ represents

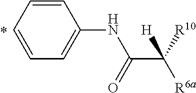

wherein * indicates the point of attachment of said group with the rest of the molecule;
R¹⁰ represents a group selected from: C₁-C₃-alkyl-, hydroxy-C₁-C₃-alkyl-, N(H)(R⁸)—C₁-C₃-alkyl-; and
R^{6a} represents a

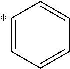

group;
wherein * indicates the point of attachment of said group with the rest of the molecule;
wherein said group is optionally substituted, one or more times, identically or differently, with a halogen atom or a methyl-group.

In another preferred embodiment, the invention relates to compounds of formula (I), wherein:
R¹ represents

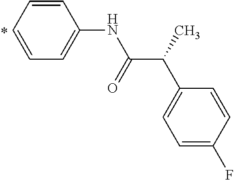

wherein * indicates the point of attachment of said group with the rest of the molecule.

In a preferred embodiment, the invention relates to compounds of formula (I), wherein:
R² represents a phenyl group or a pyridyl group
   which is substituted, one or more times, identically or differently, with a substituent selected from:
   halo-, hydroxy-, cyano-, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-,
   —C(═O)R⁹, —C(═O)O—R⁹, —OC(═O)—R⁹, —N(H)C(═O)R⁹, —N(R⁷)C(═O)R⁹, —N(H)C(═O)NR⁹R⁷, —N(R⁷)C(═O)NR⁹R⁷, —NR⁹R⁷, —C(═O)N(H)R⁹, —C(═O)NR⁹R⁷, R⁹—S—, R⁹—S(═O)—, R⁹—S(═O)₂—, —N(H)S(═O)R⁹, —N(R⁷)S(═O)R⁹, —S(═O)N(H)R⁹, —S(═O)NR⁹R⁷, —N(H)S(═O)₂R⁹, —N(R⁷)S(═O)₂R⁹, —S(═O)₂N(H)R⁹, —S(═O)₂NR⁹R⁷, —S(═O)(═NR⁹)R⁷, —S(═O)(═NR⁷)R⁹ or —N═S(═O)(R⁹)R⁷.

In another preferred embodiment, the invention relates to compounds of formula (I), wherein:
R² represents a phenyl group
   which is substituted, one or more times, identically or differently, with a substituent selected from:
   halo-, hydroxy-, cyano-, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-,
   —C(═O)R⁹, —C(═O)O—R⁹, —OC(═O)—R⁹, —N(H)C(═O)R⁹, —N(R⁷)C(═O)R⁹, —N(H)C(═O)NR⁹R⁷, —N(R⁷)C(═O)NR⁹R⁷, —NR⁹R⁷, —C(═O)N(H)R⁹, —C(═O)NR⁹R⁷, R⁹—S—, R⁹—S(═O)—, R⁹—S(═O)₂—, —N(H)S(═O)R⁹, —N(R⁷)S(═O)R⁹, —S(═O)N(H)R⁹, —S(═O)NR⁹R⁷, —N(H)S(═O)₂R⁹, —N(R⁷)S(═O)₂R⁹, —S(═O)₂N(H)R⁹, —S(═O)₂NR⁹R⁷, —S(═O)(═NR⁹)R⁷, —S(═O)(═NR⁷)R⁹ or —N═S(═O)(R⁹)R⁷.

In another preferred embodiment, the invention relates to compounds of formula (I), wherein:
R² represents a phenyl group
   which is substituted, one or more times, identically or differently, with a substituent selected from:
   halo-, cyano-, C₁-C₆-alkoxy-, hydroxy-C₁-C₆-alkyl-, —NR⁹R⁷, —C(═O)NR⁹R⁷, R⁹—S(═O)₂—.

In another preferred embodiment, the invention relates to compounds of formula (I), wherein:
R² represents

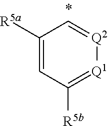

wherein * indicates the point of attachment of said group with the rest of the molecule;
R^{5a} represents a group selected from: C₁-C₄-alkoxy-, halo-C₁-C₄-alkoxy-, C₁-C₄-alkyl;
R^{5b} represents a group selected from: —C(═O)N(H)R⁹, —C(═O)NR⁹R⁷, —NR⁹R⁷, R⁹—S(═O)₂—;
Q¹ represents CH or N;
Q² represents CH or N;
   with the proviso that Q¹ represents CH if Q² represents N, and Q² represents CH if Q¹ represents N.

In another preferred embodiment, the invention relates to compounds of formula (I), wherein:
R² represents

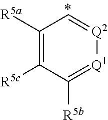

wherein * indicates the point of attachment of said group with the rest of the molecule;
R^{5a} represents a group selected from:
   C₁-C₄-alkoxy-, preferably methoxy, —CN;
R^{5b} represents a hydrogen atom or a group selected from:
   —NR⁹R⁷, —C(═O)NR⁹R⁷, R⁷—S(═O)₂—, hydroxy-C₁-C₆-alkyl-;
R^{5C} represents halo, preferably fluoro;
Q¹ represents CH or N;
Q² represents CH or N;
with the proviso that Q¹ represents CH if Q² represents N, and Q² represents CH if Q¹ represents N.

In another preferred embodiment, the invention relates to compounds of formula (I), wherein:
R² represents

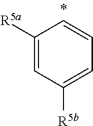

wherein * indicates the point of attachment of said group with the rest of the molecule;
R^{5a} represents a group selected from: C₁-C₄-alkoxy-, halo-C₁-C₄-alkoxy-, C₁-C₄-alkyl;
R^{5b} represents a group selected from: —C(═O)N(H)R⁹, —C(═O)NR⁹R⁷, —NR⁹R⁷, R⁹—S(═O)₂—.

In another preferred embodiment, the invention relates to compounds of formula (I), wherein:
R² represents

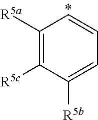

wherein * indicates the point of attachment of said group with the rest of the molecule;
R^{5a} represents a group selected from:
   C₁-C₄-alkoxy-, preferably methoxy, —CN;
R^{5b} represents a hydrogen atom or a group selected from:
   —NR⁹R⁷, —C(═O)NR⁹R⁷, R⁷—S(═O)₂—, hydroxy-C₁-C₆-alkyl-;
R^{5c} represents halo, preferably fluoro.

In another preferred embodiment, the invention relates to compounds of formula (I), wherein:
R² represents a group selected from:

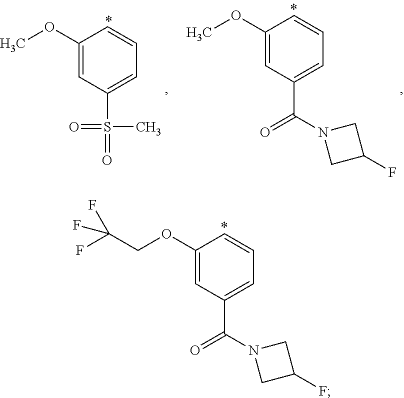

wherein * indicates the point of attachment of said group with the rest of the molecule.

In another preferred embodiment, the invention relates to compounds of formula (I), wherein:
R² represents a group selected from:

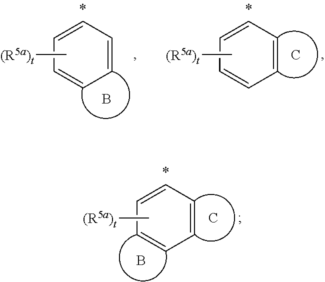

wherein * indicates the point of attachment of said group with the rest of the molecule.

In another preferred embodiment, the invention relates to compounds of formula (I), wherein:
R² represents

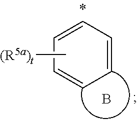

wherein * indicates the point of attachment of said group with the rest of the molecule.

In another preferred embodiment, the invention relates to compounds of formula (I), wherein R² represents:

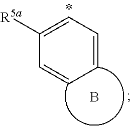

wherein * indicates the point of attachment of said group with the rest of the molecule.

In another preferred embodiment, the invention relates to compounds of formula (I), wherein R² is selected from:

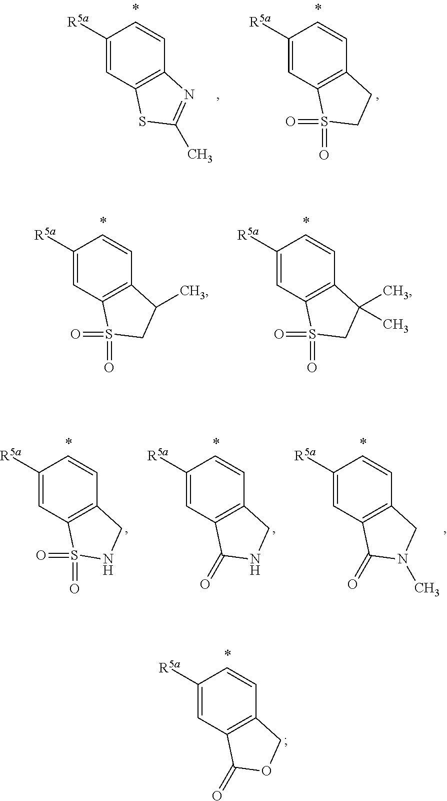

wherein * indicates the point of attachment of said groups with the rest of the molecule.

In another preferred embodiment, the invention relates to compounds of formula (I), wherein R² is selected from:

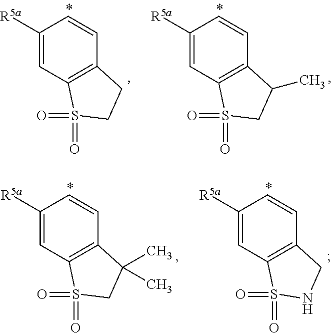

wherein * indicates the point of attachment of said groups with the rest of the molecule.

In another preferred embodiment, the invention relates to compounds of formula (I), wherein R² is selected from:

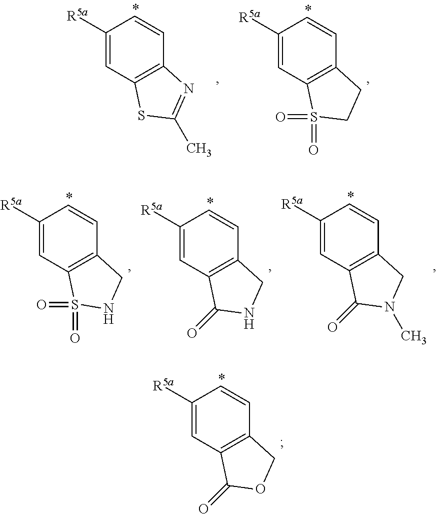

wherein * indicates the point of attachment of said groups with the rest of the molecule.

In another preferred embodiment, the invention relates to compounds of formula (I), wherein R² represents:

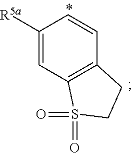

wherein * indicates the point of attachment of said groups with the rest of the molecule.

In another preferred embodiment, the invention relates to compounds of formula (I), wherein R² represents a group selected from:

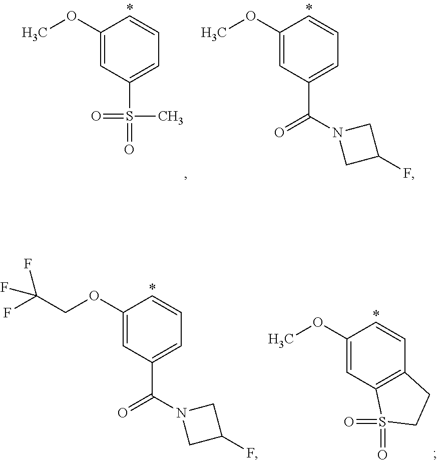

wherein * indicates the point of attachment of said group with the rest of the molecule.

In another preferred embodiment, the invention relates to compounds of formula (I), wherein B represents a 5- to 6-membered heterocyclic ring; which is optionally, one or more times, identically or differently, substituted with C₁-C₃-alkyl-, halo-C₁-C₃-alkyl-.

In another preferred embodiment, the invention relates to compounds of formula (I), wherein B represents a 5- to 6-membered heterocyclic ring.

In another preferred embodiment, the invention relates to compounds of formula (I), wherein B represents a 5-membered heterocyclic ring.

In another preferred embodiment, the invention relates to compounds of formula (I), wherein C represents a 5- to 6-membered heterocyclic ring; which is optionally substituted, one or more times, identically or differently, with halo-, —CN, —OH, C₁-C₃-alkyl-, halo-C₁-C₃-alkyl-, C₁-C₃-alkoxy-, halo-C₁-C₃-alkoxy-, hydroxy-C₁-C₃-alkyl-, C₁-C₃-alkoxy-C₁-C₃-alkyl-, halo-C₁-C₃-alkoxy-C₁-C₃-alkyl-, R⁸—(C₁-C₃-alkoxy)-, R⁸—O—, —NR⁸R⁷, R⁸—S—, R⁸—S(═O)—, R⁸—S(═O)₂—, (C₃-C₆-cycloalkyl)-(CH₂)ₙ—O—.

In another preferred embodiment, the invention relates to compounds of formula (I), wherein C represents a 5- to 6-membered heterocyclic ring; which is optionally, one or more times, identically or differently, substituted with C₁-C₃-alkyl-, halo-C₁-C₃-alkyl-.

In another preferred embodiment, the invention relates to compounds of formula (I), wherein C represents a 5- to 6-membered heterocyclic ring.

In another preferred embodiment, the invention relates to compounds of formula (I), wherein C represents a 5-membered heterocyclic ring.

In another preferred embodiment, the invention relates to compounds of formula (I), wherein:
t=1; and
R^{5a} represents a group selected from:
   halo-, C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R⁸—(C₁-C₆-alkoxy)-, R⁸—O—, R⁸—S—, R⁸—S(═O)₂—, (C₃-C₆-cycloalkyl)-(CH₂)ₙ—O—.
Preferably, R^{5a} is selected from:
   halo-, C₁-C₆-alkyl-, C₁-C₆-alkoxy-, halo-C₁-C₆-alkoxy-, C₁-C₆-alkoxy-C₁-C₆-alkyl-, (C₃-C₆-cycloalkyl)-(CH₂)ₙ—O—.
More preferably, R^{5a} is selected from:

F-, methyl-, methoxy-, ethoxy-, n-propoxy-, iso-propoxy-, cyclopropyl-O—, cyclopropyl-CH₂—O—, CH₃—O—CH₂CH₂—O—, CHF₂—O—, CF₃—O—, CF₃CH₂—O—.

In another preferred embodiment, the invention relates to compounds of formula (I), wherein:
t=1; and
R^{5a} represents a C₁-C₆-alkoxy-group.

In another preferred embodiment, the invention relates to compounds of formula (I), wherein:
t=1; and
R^{5a} represents a C₁-C₃-alkoxy-group.

In another preferred embodiment, the invention relates to compounds of formula (I), wherein:
t=1; and
R^{5a} represents a halo-C₁-C₆-alkoxy-group.

In another preferred embodiment, the invention relates to compounds of formula (I), wherein:
t=1; and
R^{5a} represents a halo-C₁-C₃-alkoxy-group.

In another preferred embodiment, the invention relates to compounds of formula (I), wherein:
t=1; and
R^{5a} represents a (C₃-C₆-cycloalkyl)-(CH₂)ₙ—O— group.

In another preferred embodiment, the invention relates to compounds of formula (I), supra, wherein t=1, and R^{5a} represents a group selected from:
C₁-C₃-alkoxy-, halo-C₁-C₃-alkoxy-, C₁-C₃-alkyl-.

In another preferred embodiment, the invention relates to compounds of formula (I), supra, wherein t=1, and R^{5a} represents a group selected from:
C₁-C₂-alkoxy-, halo-C₁-C₂-alkoxy-, C₁-C₂-alkyl-.

In another preferred embodiment, the invention relates to compounds of formula (I), supra, wherein t=1, and R^{5a} represents a group selected from:
C₁-C₃-alkoxy-, halo-C₁-C₃-alkoxy-.

In another preferred embodiment, the invention relates to compounds of formula (I), supra, wherein t=1, and R^{5a} represents a group selected from:
C₁-C₂-alkoxy-, halo-C₁-C₂-alkoxy-.

In another preferred embodiment, the invention relates to compounds of formula (I), supra, wherein t=1, and R^{5a} represents a methoxy- or ethoxy-group which is optionally substituted, one or more times, identically or differently, with a halogen atom. The preferred halogen atom is F.

In another preferred embodiment, the invention relates to compounds of formula (I), supra, wherein t=1, and R^{5a} represents a group selected from: methoxy-, ethoxy-, F₃C—CH₂—O—.

In another preferred embodiment, the invention relates to compounds of formula (I), supra, wherein t=1, and R^{5a} represents a group selected from: methoxy-, F₃C—CH₂—O—.

In another preferred embodiment, the invention relates to compounds of formula (I), supra, wherein t=1, and R^{5a} represents methoxy-.

In another preferred embodiment, the invention relates to compounds of formula (I), supra, wherein t=1, and R^{5a} represents F₃C—CH₂—O—.

In another preferred embodiment, the invention relates to compounds of formula (I), supra, wherein R^{5b} represents a group selected from:
—C(═O)N(H)R⁹, —C(═O)NR⁹R⁷, —NR⁹R⁷, R⁹—S(═O)₂—.

In another preferred embodiment, the invention relates to compounds of formula (I), supra, wherein R^{5b} represents a group selected from:
—C(═O)N(H)R⁹, —C(═O)NR⁹R⁷.

In another preferred embodiment, the invention relates to compounds of formula (I), supra, wherein R^{5b} represents a group:
—NR⁹R⁷.

In another preferred embodiment, the invention relates to compounds of formula (I), supra, wherein R^{5b} represents a group:
R⁹—S(═O)₂—.

In another preferred embodiment, the invention relates to compounds of formula (I), supra, wherein R^{5b} represents a hydrogen atom or a group selected from:
—NR⁹R⁷, —C(═O)NR⁹R⁷, R⁷—S(═O)₂—, hydroxy-C₁-C₆-alkyl-.

In another preferred embodiment, the invention relates to compounds of formula (I), supra, wherein R^{5b} represents a hydrogen atom.

In another preferred embodiment, the invention relates to compounds of formula (I), supra, wherein R^{5b} represents a group:
hydroxy-C₁-C₆-alkyl-.

In another preferred embodiment, the invention relates to compounds of formula (I), wherein R^{5c} represents halo.

In another preferred embodiment, the invention relates to compounds of formula (I), wherein R^{5c} represents fluoro.
R⁶ represents a group selected from:
   C₃-C₆-cycloalkyl-, —(CH₂)_{q}—(C₃-C₆-cycloalkyl),
   —(CH₂)_{q}-(3- to 10-membered heterocycloalkyl), —(CH₂)_{q}-aryl, or —(CH₂)_{q}-heteroaryl;
   said group being optionally substituted, one or more times, identically or differently, with a substituent selected from:
   halo-, C₁-C₆-alkyl-, halo-C₁-C₆-alkyl-, C₁-C₆-alkoxy-, halo-C₁-C₆-alkoxy-.

In another preferred embodiment, the invention relates to compounds of formula (I), wherein:
R⁶ represents —(CH₂)_{q}—(C₃-C₆-cycloalkyl);
   said group being optionally substituted, one or more times, identically or differently, with a substituent selected from:
   halo-, C₁-C₆-alkyl-, halo-C₁-C₆-alkyl-, C₁-C₆-alkoxy-, halo-C₁-C₆-alkoxy-.

In another preferred embodiment, the invention relates to compounds of formula (I), wherein:
R⁶ represents —(CH₂)_{q}-aryl;
   said group being optionally substituted, one or more times, identically or differently, with a substituent selected from:
   halo-, C₁-C₆-alkyl-, halo-C₁-C₆-alkyl-, C₁-C₆-alkoxy-, halo-C₁-C₆-alkoxy-.

In another preferred embodiment, the invention relates to compounds of formula (I), wherein:
R⁶ represents a group selected from:
   —(CH₂)_{q}— (C₃-C₆-cycloalkyl), —(CH₂)_{q}-aryl;
   said group being optionally substituted, one or more times, identically or differently, with a substituent selected from:
   halo-, C₁-C₆-alkyl-.

In another preferred embodiment, the invention relates to compounds of formula (I), wherein:
R⁶ represents —(CH₂)_{q}—(C₃-C₆-cycloalkyl);
   said group being optionally substituted, one or more times, identically or differently, with a substituent selected from:
   halo-, C₁-C₆-alkyl-.

In another preferred embodiment, the invention relates to compounds of formula (I), wherein:
R⁶ represents —(CH₂)_{q}-aryl;
   said group being optionally substituted, one or more times, identically or differently, with a substituent selected from:
   halo-, C₁-C₆-alkyl-.

In another preferred embodiment, the invention relates to compounds of formula (I), wherein:
R⁶ represents C₁-C₆-alkyl, —(CH₂)_{q}—(C₃-C₆-cycloalkyl) or —(CH₂)_{q}-aryl;
   said group being optionally substituted, one or more times, identically or differently, with halo-.

In another preferred embodiment, the invention relates to compounds of formula (I), supra, wherein R⁶ represents a group selected from:

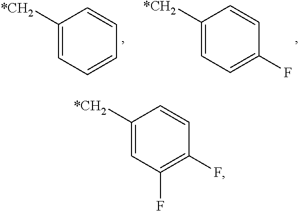

wherein * indicates the point of attachment of said group with the rest of the molecule.

In another preferred embodiment, the invention relates to compounds of formula (I), supra, wherein R⁶ represents a group selected from:

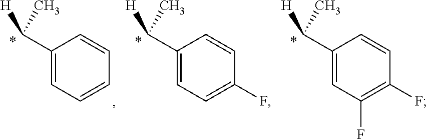

wherein * indicates the point of attachment of said group with the rest of the molecule.

In another preferred embodiment, the invention relates to compounds of formula (I), supra, wherein R⁶ represents a group:

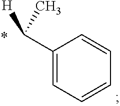

wherein * indicates the point of attachment of said group with the rest of the molecule.

In another preferred embodiment, the invention relates to compounds of formula (I), supra, wherein R⁶ represents a group:

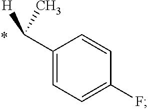

wherein * indicates the point of attachment of said group with the rest of the molecule.

In another preferred embodiment, the invention relates to compounds of formula (I), supra, wherein R⁶ represents a group:

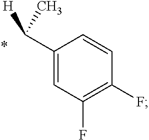

wherein * indicates the point of attachment of said group with the rest of the molecule.

In another preferred embodiment, the invention relates to compounds of formula (I), wherein:
R⁷ represents a hydrogen atom, a C₁-C₆-alkyl-, or C₃-C₆-cycloalkyl-group.

Preferably, R⁷ represents a hydrogen atom or a C₁-C₆-alkyl-group. More preferably, R⁷ represents a hydrogen atom.

In another preferred embodiment, the invention relates to compounds of formula (I), wherein:
R⁶ and R⁷,
   together with the nitrogen atom to which they are attached, represent a 3- to 10-membered heterocycloalkyl-group.

In another preferred embodiment, the invention relates to compounds of formula (I), wherein:
R⁸ represents a hydrogen atom or a C₁-C₆-alkyl-group.
Preferably, R⁸ represents a C₁-C₆-alkyl-group

In another preferred embodiment, the invention relates to compounds of formula (I), wherein:
R⁹ represents a C₁-C₆-alkyl-group.

In another preferred embodiment, the invention relates to compounds of formula (I), wherein:
R⁹ and R⁷,
   together with the nitrogen atom to which they are attached, represent a 3- to 10-membered heterocycloalkyl-group which is optionally substituted with a halogen atom, preferably with fluoro.

In another preferred embodiment, the invention relates to compounds of formula (I), wherein:
R⁹ and R⁷,
   together with the nitrogen atom to which they are attached, represent a group selected from:

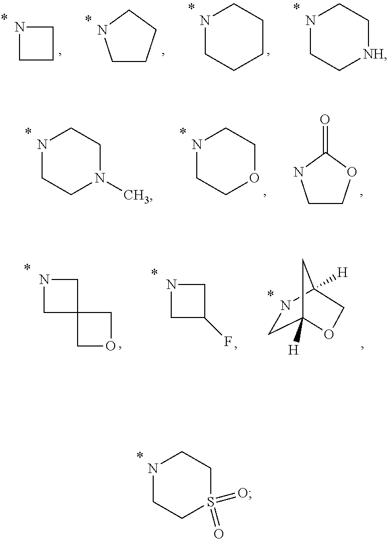

wherein * indicates the point of attachment of said group with the rest of the molecule.

In another preferred embodiment, the invention relates to compounds of formula (I), wherein:
R⁹ and R⁷,
   together with the nitrogen atom to which they are attached, represent a group selected from:

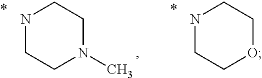

wherein * indicates the point of attachment of said group with the rest of the molecule.

In another preferred embodiment, the invention relates to compounds of formula (I), wherein:
R⁹ and R⁷,
   together with the nitrogen atom to which they are attached, represent a group selected from:

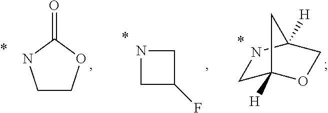

wherein * indicates the point of attachment of said group with the rest of the molecule.

In another preferred embodiment, the invention relates to compounds of formula (I), wherein Q¹ represents CH and Q² represents CH.

In another preferred embodiment, the invention relates to compounds of formula (I), wherein:
n, m, p
   represent, independently from each other, an integer of 0, 1, 2 or 3.

In another preferred embodiment, the invention relates to compounds of formula (I), wherein:
q represents an integer of 1 or 2.
Preferably, q is 1.

In another preferred embodiment, the invention relates to compounds of formula (I), wherein:
t represents an integer of 1 or 2.
Preferably, t represents 1.

It is to be understood that the present invention relates also to any combination of the preferred embodiments described above.

Some examples of combinations are given hereinafter. However, the invention is not limited to these combinations.

In a preferred embodiment, the invention relates to compounds of formula (I):

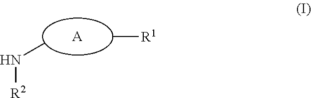

in which
A is selected from:

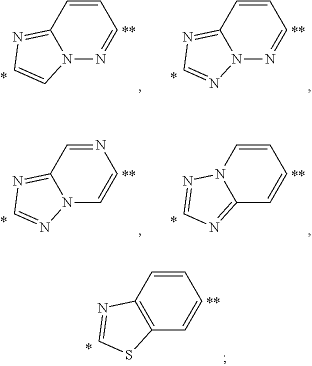

wherein * represents the point of attachment to the nitrogen atom and ** represents the point of attachment to the R¹ group;
R¹ represents a phenyl-group
   which is substituted, one or more times, identically or differently, with a substituent selected from:
   —OH, —N(H)C(═O)R⁶, —NH₂, —C(═O)N(H)R⁶;
   and
   which is optionally substituted, one or more times, identically or differently, with a C₁-C₆-alkyl-group;
R² represents a hydrogen atom or a phenyl-group; said phenyl-group being substituted, one or more times, identically or differently, with a substituent selected from:
   halo-, cyano-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, —NR⁹R⁷, —C(═O)NR⁹R⁷, R⁹—S(═O)₂—;
or
R² represents:

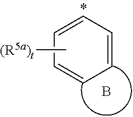

wherein * indicates the point of attachment of said group with the rest of the molecule;
B represents a 5- to 6-membered heterocyclic ring; which is optionally, one or more times, identically or differently, substituted with C₁-C₃-alkyl-, halo-C₁-C₃-alkyl-.
R^{5a} represents a group selected from:
   halo-, C₁-C₆-alkyl-, halo-C₁-C₆-alkyl-, C₁-C₆-alkoxy-, halo-C₁-C₆-alkoxy-;
R⁶ represents a group selected from:

C₁-C₆-alkyl-, C₃-C₆-cycloalkyl-, —(CH₂)_{q}—(C₃-C₆-cycloalkyl), —(CH₂)_{q}-aryl;
said group being optionally substituted, one or more times, identically or differently, with a substituent selected from:
fluoro-;
R⁹ and R⁷,
   together with the nitrogen atom to which they are attached, represent a 3- to 10-membered heterocycloalkyl-group;
q represents an integer of 1;
and
t represents an integer of 1;
or a stereoisomer, a tautomer, an N-oxide, a hydrate, a solvate, or a salt thereof, or a mixture of same.

In another preferred embodiment, the invention relates to compounds of formula (I):

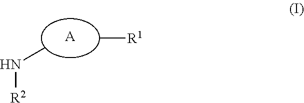

in which:
A is selected from:

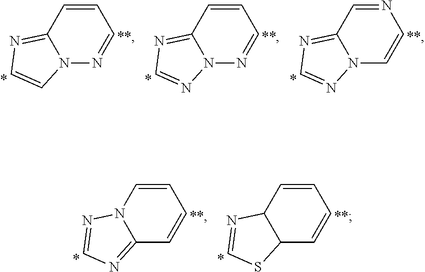

wherein * represents the point of attachment to the nitrogen atom and ** represents the point of attachment to the R¹ group;
R¹ represents a phenyl-group
   which is substituted, one or more times, identically or differently, with a substituent selected from:
   —OH, —N(H)C(═O)R⁶, —NH₂, —C(═O)N(H)R⁶;
   and
   which is optionally substituted, one or more times, identically or differently, with a C₁-C₆-alkyl-group;
R² represents a hydrogen atom or a phenyl-group; said phenyl-group being substituted, one or more times, identically or differently, with a substituent selected from:
   halo-, cyano-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, —NR⁹R⁷, —C(═O)NR⁹R⁷, R⁹—S(═O)₂—;
or
R² represents:

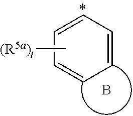

wherein * indicates the point of attachment of said group with the rest of the molecule;
B represents a 5- to 6-membered heterocyclic ring; which is optionally, one or more times, identically or differently, substituted with C₁-C₃-alkyl-, halo-C₁-C₃-alkyl-.
R^{5a} represents a group selected from:
   halo-, C₁-C₆-alkyl-, halo-C₁-C₆-alkyl-, C₁-C₆-alkoxy-, halo-C₁-C₆-alkoxy-;
R⁶ represents a group selected from:
   C₁-C₆-alkyl-, C₃-C₆-cycloalkyl-, —(CH₂)_{q}—(C₃-C₆-cycloalkyl), —(CH₂)_{q}-aryl;
   said group being optionally substituted, one or more times, identically or differently, with a substituent selected from:
   fluoro-, methyl-;
R⁹ and R⁷,
   together with the nitrogen atom to which they are attached, represent a 3- to 10-membered heterocycloalkyl-group;
q represents an integer of 1;
and
t represents an integer of 1;
or a stereoisomer, a tautomer, an N-oxide, a hydrate, a solvate, or a salt thereof, or a mixture of same.

In another preferred embodiment, the invention relates to compounds of formula (I):

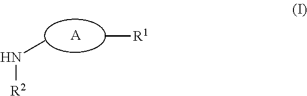

in which:
A represents

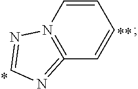

wherein * represents the point of attachment to the nitrogen atom and ** represents the point of attachment to the R¹ group;
R¹ represents a phenyl-group
   which is substituted one time with a substituent selected from:
   —N(H)C(═O)R⁶, —C(═O)N(H)R⁶;
R² represents a phenyl-group; said phenyl-group being substituted, one or more times, identically or differently, with a substituent selected from:
   halo-, hydroxy-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, —NR⁹R⁷, —C(═O)N(H)R⁹, —C(═O)NR⁹R⁷, R⁹—S—, R⁹—S(═O)—, R⁹—S(═O)₂—, —N(H)S(═O)R⁹, —N(R⁷)S(═O)R⁹, —S(═O)N(H)R⁹, —S(═O)NR⁹R⁷, —N(H)S(═O)₂R⁹, —N(R⁷)S(═O)₂R⁹, —S(═O)₂N(H)R⁹, —S(═O)₂NR⁹R⁷, —S(═O)(═NR⁹)R⁷, —S(═O)(═NR⁷)R⁹ or —N═S(═O)(R⁹)R⁷;
or
R² represents

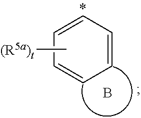

wherein * indicates the point of attachment of said group with the rest of the molecule;
B represents a 4- to 6-membered heterocyclic ring; which is optionally substituted, one or more times, identically or differently, with halo-, —CN, —OH, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R⁸—(C₁-C₆-alkoxy)-, R⁸—O—, —NR⁸R⁷, R⁸—S—, R⁸—S(═O)—, R⁸—S(═O)₂—, (C₃-C₆-cycloalkyl)-(CH₂)ₙ—O—;
each R^{5a}
   independently represents a group selected from:
   halo-, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R⁸—(C₁-C₆-alkoxy)-, R⁸—O—, —NR⁸R⁷, R⁸—S—, R⁸—S(═O)—, R⁸—S(═O)₂—, (C₃-C₆-cycloalkyl)-(CH₂)ₙ—O—;
R⁶ represents a group selected from:
   C₁-C₆-alkyl-, C₃-C₆-cycloalkyl-, 3- to 10-membered heterocycloalkyl-, aryl-, heteroaryl-, —(CH₂)_{q}—(C₃-C₆-cycloalkyl), —(CH₂)_{q}-heteroaryl, —(CH₂)_{q}-(3- to 10-membered heterocycloalkyl), —(CH₂)_{q}-aryl;
   said group being optionally substituted, one or more times, identically or differently, with a substituent selected from:
   halo-, hydroxy-, cyano-, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R⁸—(C₁-C₆-alkyl)-, R⁸—(CH₂)ₙ(CHOH)(CH₂)ₘ—, R⁸—(C₁-C₆-alkoxy)-, R⁸—(CH₂)ₙ(CHOH)(CH₂)ₚ—O—, R⁸—(C₁-C₆-alkoxy-C₁-C₆-alkyl)-, R⁸—(C₁-C₆-alkoxy-C₁-C₆-alkyl)-O—, aryl-, R⁸—O—, —C(═O)R⁸, —C(═O)O—R⁸, —OC(═O)—R⁸, —N(H)C(═O)R⁸, —N(R⁷)C(═O)R⁸, —N(H)C(═O)NR⁸R⁷, —N(R⁷)C(═O)NR⁸R⁷, —NR⁸R⁷, —C(═O)N(H)R⁸, —C(═O)NR⁸R⁷, R⁸—S—, R⁸—S(═O)—, R⁸—S(═O)₂—, —N(H)S(═O)R⁸, —N(R⁷)S(═O)R⁸, —S(═O)N(H)R⁸, —S(═O)NR⁸R⁷, —N(H)S(═O)₂R⁸, —N(R⁷)S(═O)₂R⁸, —S(═O)₂N(H)R⁸, —S(═O)₂NR⁸R⁷, —S(═O)(═NR⁸)R⁷, —S(═O)(═NR⁷)R⁸, —N═S(═O)(R⁸)R⁷;
R⁷ represents a hydrogen atom, a C₁-C₆-alkyl-, or C₃-C₆-cycloalkyl-group;
R⁸ represents a hydrogen atom, a C₁-C₆-alkyl- or C₃-C₆-cycloalkyl-group;
R⁹ represents a C₁-C₆-alkyl-group;
or
R⁹ and R⁷,
   together with the nitrogen atom to which they are attached, represent a 3- to 10-membered heterocycloalkyl-group;
n, m, p
   represent, independently from each other, an integer of 0, 1, 2, 3, 4, or 5;
q represents an integer of 1;
and
t represents an integer of 0, 1 or 2;
or a stereoisomer, a tautomer, an N-oxide, a hydrate, a solvate, or a salt thereof, or a mixture of same.

In another preferred embodiment, the invention relates to compounds of formula (I):

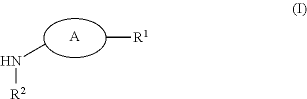

in which:
A is selected from:

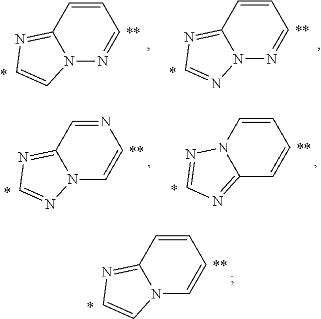

wherein * represents the point of attachment to the nitrogen atom and ** represents the point of attachment to the R¹ group;
R¹ represents

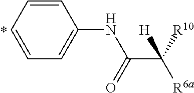

wherein * indicates the point of attachment of said group with the rest of the molecule;
R² represents a phenyl group which is substituted, one or more times, identically or differently, with a substituent selected from:
   halo-, cyano-, C₁-C₆-alkoxy-, hydroxy-C₁-C₆-alkyl-, —NR⁹R⁷, —C(═O)NR⁹R⁷, R⁹—S(═O)₂—;
or
R² represents:

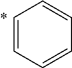

wherein * indicates the point of attachment of said group with the rest of the molecule;
R^{5a} represents a C₁-C₃-alkoxy- or a halo-C₁-C₃-alkoxy-group;
R^{6a} represents a

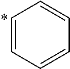

group;
wherein * indicates the point of attachment of said group with the rest of the molecule; wherein said group is optionally substituted, one or more times, identically or differently, with a halogen atom or a methyl-group;
R⁷ represents a hydrogen atom, a C₁-C₆-alkyl-, or C₃-C₆-cycloalkyl-group;
R⁸ represents a hydrogen atom, a C₁-C₆-alkyl- or C₃-C₆-cycloalkyl-group;
R⁹ represents a C₁-C₆-alkyl-group;
or
R⁹ and R⁷,
   together with the nitrogen atom to which they are attached, represent a 3- to 10-membered heterocycloalkyl-group;
and
R¹⁰ represents a group selected from: C₁-C₃-alkyl-, hydroxy-C₁-C₃-alkyl-, N(H)(R⁸)—C₁-C₃-alkyl-;
or a stereoisomer, a tautomer, an N-oxide, a hydrate, a solvate, or a salt thereof, or a mixture of same.

In another preferred embodiment, the invention relates to compounds of formula (I):

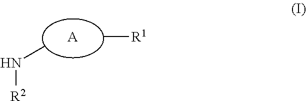

in which:
A represents

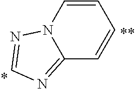

wherein * represents the point of attachment to the nitrogen atom and ** represents the point of attachment to the R¹ group;
R¹ represents

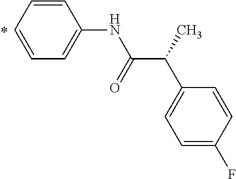

wherein * indicates the point of attachment of said group with the rest of the molecule;
R² represents a group selected from:

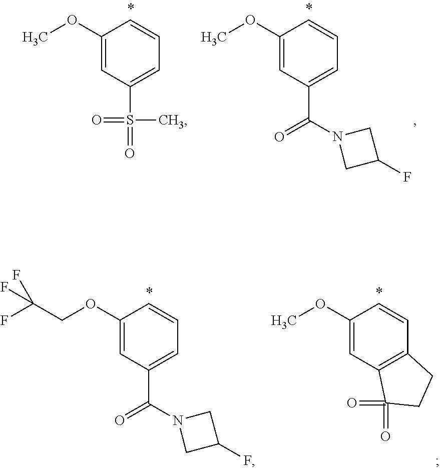

wherein * indicates the point of attachment of said group with the rest of the molecule;
or a stereoisomer, a tautomer, an N-oxide, a hydrate, a solvate, or a salt thereof, or a mixture of same.

The present invention covers compounds of general formula (I) which are disclosed in the Example section of this text, infra.

In an embodiment of the above-mentioned embodiments of the above-mentioned aspects, the invention relates to a stereoisomer, a tautomer, an N-oxide, a hydrate, a solvate, or a salt thereof, or a mixture of same, of any of the compounds of formula (I).

In accordance with another aspect, the present invention covers methods of preparing compounds of the present invention, said methods comprising the steps as described in the Experimental Section herein.

This invention also relates to pharmaceutical compositions containing one or more compounds of the present invention. These compositions can be utilised to achieve the desired pharmacological effect by administration to a patient in need thereof. A patient, for the purpose of this invention, is a mammal, including a human, in need of treatment for the particular condition or disease. Therefore, the present invention includes pharmaceutical compositions that are comprised of a pharmaceutically acceptable carrier and a pharmaceutically effective amount of a compound, or salt thereof, of the present invention. A pharmaceutically acceptable carrier is preferably a carrier that is relatively non-toxic and innocuous to a patient at concentrations consistent with effective activity of the active ingredient so that any side effects ascribable to the carrier do not vitiate the beneficial effects of the active ingredient. A pharmaceutically effective amount of compound is preferably that amount which produces a result or exerts an influence on the particular condition being treated. The compounds of the present invention can be administered with pharmaceutically-acceptable carriers well known in the art using any effective conventional dosage unit forms, including immediate, stow and timed release preparations, orally, parenterally, topically, nasally, ophthalmically, optically, sublingually, rectally, vaginally, and the like.

The compounds of this invention can be administered as the sole pharmaceutical agent or in combination with one or more other pharmaceutical agents where the combination causes no unacceptable adverse effects. The present invention relates also to such combinations. For example, the compounds of this invention can be combined with known anti-hyper-proliferative or other indication agents, and the like, as well as with admixtures and combinations thereof. Other indication agents include, but are not limited to, anti-angiogenic agents, mitotic inhibitors, alkylating agents, anti-metabolites, DNA-intercalating antibiotics, growth factor inhibitors, cell cycle inhibitors, enzyme inhibitors, toposisomerase inhibitors, biological response modifiers, or anti-hormones.

Preferred additional pharmaceutical agents are: 131I-chTNT, abarelix, abiraterone, aclarubicin, aldesleukin, alemtuzumab, alitretinoin, altretamine, aminoglutethimide, amrubicin, amsacrine, anastrozole, arglabin, arsenic trioxide, asparaginase, azacitidine, basiliximab, BAY 80-6946, BAY 1000394, BAY 86-9766 (RDEA 119), belotecan, bendamustine, bevacizumab, bexarotene, bicalutamide, bisantrene, bleomycin, bortezomib, buserelin, busulfan, cabazitaxel, calcium folinate, calcium levofolinate, capecitabine, carboplatin, carmofur, carmustine, catumaxomab, celecoxib, celmoleukin, cetuximab, chlorambucil, chlormadinone, chlormethine, cisplatin, cladribine, clodronic acid, clofarabine, crisantaspase, cyctophosphamide, cyproterone, cytarabine, dacarbazine, dactinomycin, darbepoetin alfa, dasatinib, daunorubicin, decitabine, degarelix, denileukin diftitox, denosumab, deslorelin, dibrospidium chloride, docetaxel, doxifluridine, doxorubicin, doxorubicin+estrone, eculizumab, edrecolomab, elliptinium acetate, eltrombopag, endostatin, enocitabine, epirubicin, epitiostanol, epoetin alfa, epoetin beta, eptaplatin, eribulin, erlotinib, estradiol, estramustine, etoposide, everolimus, exemestane, fadrozole, filgrastim, fludarabine, fluorouracil, flutamide, formestane, fotemustine, fulvestrant, gallium nitrate, ganirelix, gefitinib, gemcitabine, gemtuzumab, glutoxim, goserelin, histamine dihydrochloride, histrelin, hydroxycarbamide, I-125 seeds, ibandronic acid, ibritumomab tiuxetan, idarubicin, ifosfamide, imatinib, imiquimod, improsutfan, interferon alfa, interferon beta, interferon gamma, ipilimumab, irinotecan, ixabepilone, Lanreotide, lapatinib, lenalidomide, lenograstim, lentinan, letrozole, leuprorelin, levamisole, lisuride, lobaplatin, lomustine, lonidamine, masoprocol, medroxyprogesterone, megestrol, melphalan, mepitiostane, mercaptopurine, methotrexate, methoxsalen, Methyl aminolevulinate, methyltestosterone, mifamurtide, miltefosine, miriplatin, mitobronitol, mitoguazone, mitolactol, mitomycin, mitotane, mitoxantrone, nedaplatin, nelarabine, nilotinib, nilutamide, nimotuzumab, nimustine, nitracrine, ofatumumab, omeprazole, oprelvekin, oxaliplatin, p53 gene therapy, paclitaxel, palifermin, palladium-103 seed, pamidronic acid, panitumumab, pazopanib, pegaspargase, PEG-epoetin beta (methoxy PEG-epoetin beta), pegfilgrastim, peginterferon alfa-2b, pemetrexed, pentazocine, pentostatin, peplomycin, perfosfamide, picibanil, pirarubicin, plerixafor, plicamycin, poliglusam, polyestradiol phosphate, polysaccharide-K, porfimer sodium, pralatrexate, prednimustine, procarbazine, quinagolide, raloxifene, raltitrexed, ranimustine, razoxane, regorafenib, risedronic acid, rituximab, romidepsin, romiplostim, sargramostim, sipuleucel-T, sizofiran, sobuzoxane, sodium glycididazole, sorafenib, streptozocin, sunitinib, talaporfin, tamibarotene, tamoxifen, tasonermin, teceleukin, tegafur, tegafur+gimeracil+oteracil, temoporfin, temozolomide, temsirolimus, teniposide, testosterone, tetrofosmin, thalidomide, thiotepa, thymalfasin, tioguanine, tocilizumab, topotecan, toremifene, tositumomab, trabectedin, trastuzumab, treosulfan, tretinoin, trilostane, triptorelin, trofosfamide, tryptophan, ubenimex, valrubicin, vandetanib, vapreotide, vemurafenib, vinblastine, vincristine, vindesine, vinflunine, vinorelbine, vorinostat, vorozole, yttrium-90 glass microspheres, zinostatin, zinostatin stimalamer, zoledronic acid, zorubicin.

Optional anti-hyper-proliferative agents which can be added to the composition include but are not limited to compounds listed on the cancer chemotherapy drug regimens in the 11^{th} Edition of the Merck Index, (1996), which is hereby incorporated by reference, such as asparaginase, bleomycin, carboplatin, carmustine, chlorambucil, cisplatin, colaspase, cyclophosphamide, cytarabine, dacarbazine, dactinomycin, daunorubicin, doxorubicin (adriamycine), epirubicin, etoposide, 5-fluorouracil, hexamethylmelamine, hydroxyurea, ifosfamide, irinotecan, leucovorin, lomustine, mechlorethamine, 6-mercaptopurine, mesna, methotrexate, mitomycin C, mitoxantrone, prednisolone, prednisone, procarbazine, raloxifen, streptozocin, tamoxifen, thioguanine, topotecan, vinblastine, vincristine, and vindesine.

Other anti-hyper-proliferative agents suitable for use with the composition of the invention include but are not limited to those compounds acknowledged to be used in the treatment of neoplastic diseases in Goodman and Gilman's The Pharmacological Basis of Therapeutics (Ninth Edition), editor Motinoff et al., publ. by McGraw-Hill, pages 1225-1287, (1996), which is hereby incorporated by reference, such as aminoglutethimide, L-asparaginase, azathioprine, 5-azacytidine cladribine, busulfan, diethylstilbestrol, 2′,2′-difluorodeoxycytidine, docetaxel, erythrohydroxynonyl adenine, ethinyl estradiol, 5-fluorodeoxyuridine, 5-fluorodeoxyuridine monophosphate, fludarabine phosphate, fluoxymesterone, flutamide, hydroxyprogesterone caproate, idarubicin, interferon, medroxyprogesterone acetate, megestrol acetate, melphalan, mitotane, paclitaxel, pentostatin, N-phosphonoacetyl-L-aspartate (PALA), plicamycin, semustine, teniposide, testosterone propionate, thiotepa, trimethylmelamine, uridine, and vinorelbine.

Other anti-hyper-proliferative agents suitable for use with the composition of the invention include but are not limited to other anti-cancer agents such as epothilone and its derivatives, irinotecan, raloxifen and topotecan.

The compounds of the invention may also be administered in combination with protein therapeutics. Such protein therapeutics suitable for the treatment of cancer or other angiogenic disorders and for use with the compositions of the invention include, but are not limited to, an interferon (e.g., interferon .alpha., .beta., or .gamma.) supraagonistic monoclonal antibodies, Tuebingen, TRP-1 protein vaccine, Colostrinin, anti-FAP antibody, YH-16, gemtuzumab, infliximab, cetuximab, trastuzumab, denileukin diftitox, rituximab, thymosin alpha 1, bevacizumab, mecasermin, mecasermin rinfabate, oprelvekin, natalizumab, rhMBL, MFE-CP1+ZD-2767-P, ABT-828, ErbB2-specific immunotoxin, SGN-35, MT-103, rinfabate, AS-1402, B43-genistein, L-19 based radioimmunotherapeutics, AC-9301, NY-ESO-1 vaccine, IMC-1C11, CT-322, rhCC10, r(m)CRP, MORAb-009, aviscumine, MDX-1307, Her-2 vaccine, APC-8024, NGR-hTNF, rhH1.3, IGN-311, Endostatin, volociximab, PRO-1762, lexatumumab, SGN-40, pertuzumab, EMD-273063, L19-IL-2 fusion protein, PRX-321, CNTO-328, MDX-214, tigapotide, CAT-3888, Labetuzumab, alpha-particle-emitting radioisotope-llinked lintuzumab, EM-1421, HyperAcute vaccine, tucotuzumab cetmoteukin, galiximab, HPV-16-E7, Javelin—prostate cancer, Javelin—melanoma, NY-ESO-1 vaccine, EGF vaccine, CYT-004-MelQbG10, WT1 peptide, oregovomab, ofatumumab, zalutumumab, cintredekin besudotox, WX-G250, Albuferon, aflibercept, denosumab, vaccine, CTP-37, efungumab, or 131I-chTNT-1/B. Monoclonal antibodies useful as the protein therapeutic include, but are not limited to, muromonab-CD3, abciximab, edrecolomab, daclizumab, gentuzumab, alemtuzumab, ibritumomab, cetuximab, bevicizumab, efalizumab, adalimumab, omalizumab, muromomab-CD3, rituximab, daclizumab, trastuzumab, palivizumab, basiliximab, and infliximab.

Generally, the use of cytotoxic and/or cytostatic agents in combination with a compound or composition of the present invention will serve to:
(1) yield better efficacy in reducing the growth of a tumor or even eliminate the tumor as compared to administration of either agent alone,
(2) provide for the administration of lesser amounts of the administered chemotherapeutic agents,
(3) provide for a chemotherapeutic treatment that is well tolerated in the patient with fewer deleterious pharmacological complications than observed with single agent chemotherapies and certain other combined therapies,
(4) provide for treating a broader spectrum of different cancer types in mammals, especially humans,
(5) provide for a higher response rate among treated patients,
(6) provide for a longer survival time among treated patients compared to standard chemotherapy treatments,
(7) provide a longer time for tumor progression, and/or
(8) yield efficacy and tolerability results at least as good as those of the agents used alone, compared to known instances where other cancer agent combinations produce antagonistic effects.

In accordance with another aspect therefore, the present invention covers a compound of general formula (I), or a stereoisomer, a tautomer, an N-oxide, a hydrate, a solvate, or a salt thereof, particularly a pharmaceutically acceptable salt thereof, or a mixture of same, as described and defined herein, for use in the treatment or prophylaxis of a disease, as mentioned supra.

Another particular aspect of the present invention is therefore the use of a compound of general formula (I), described supra, or a stereoisomer, a tautomer, an N-oxide, a hydrate, a solvate, or a salt thereof, particularly a pharmaceutically acceptable salt thereof, or a mixture of same, for the prophylaxis or treatment of a disease.

Another particular aspect of the present invention is therefore the use of a compound of general formula (I) described supra for manufacturing a pharmaceutical composition for the treatment or prophylaxis of a disease.

The diseases referred to in the two preceding paragraphs are diseases of uncontrolled cell growth, proliferation and/or survival, inappropriate cellular immune responses, or inappropriate cellular inflammatory responses, or diseases which are accompanied with uncontrolled cell growth, proliferation and/or survival, inappropriate cellular immune responses, or inappropriate cellular inflammatory responses, particularly in which the uncontrolled cell growth, proliferation and/or survival, inappropriate cellular immune responses, or inappropriate cellular inflammatory responses is mediated by Mps-1, such as, for example, haematological tumours, solid tumours, and/or metastases thereof, e.g. Leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof.

The term “inappropriate” within the context of the present invention, in particular in the context of “inappropriate cellular immune responses, or inappropriate cellular inflammatory responses”, as used herein, is to be understood as preferably meaning a response which is less than, or greater than normal, and which is associated with, responsible for, or results in, the pathology of said diseases.

Preferably, the use is in the treatment or prophylaxis of diseases, wherein the diseases are haemotological tumours, solid tumours and/or metastases thereof.

The present invention relates to a method for using the compounds of the present invention and compositions thereof, to treat mammalian hyper-proliferative disorders. Compounds can be utilized to inhibit, block, reduce, decrease, etc., cell proliferation and/or cell division, and/or produce apoptosis. This method comprises administering to a mammal in need thereof, including a human, an amount of a compound of this invention, or a pharmaceutically acceptable salt, isomer, polymorph, metabolite, hydrate, solvate or ester thereof; etc. which is effective to treat the disorder. Hyper-proliferative disorders include but are not limited, e.g., psoriasis, keloids, and other hyperplasias affecting the skin, benign prostate hyperplasia (BPH), solid tumors, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukemias.

Examples of breast cancer include, but are not limited to invasive ductal carcinoma, invasive lobular carcinoma, ductal carcinoma in situ, and lobular carcinoma in situ.

Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypothalamic glioma, cerebellar and cerebral astrocytoma, medulloblastoma, ependymoma, as well as neuroectodermal and pineal tumor.

Tumors of the male reproductive organs include, but are not limited to prostate and testicular cancer. Tumors of the female reproductive organs include, but are not limited to endometrial, cervical, ovarian, vaginal, and vulvar cancer, as well as sarcoma of the uterus.

Tumors of the digestive tract include, but are not limited to anal, colon, colorectal, esophageal, gallbladder, gastric, pancreatic, rectal, small-intestine, and salivary gland cancers.

Tumors of the urinary tract include, but are not limited to bladder, penile, kidney, renal pelvis, ureter, urethral and human papillary renal cancers.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to laryngeal, hypopharyngeal, nasopharyngeal, oropharyngeal cancer, lip and oral cavity cancer and squamous cell. Lymphomas include, but are not limited to AIDS-related lymphoma, non-Hodgkin's Lymphoma, cutaneous T-cell Lymphoma, Burkitt Lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

These disorders have been well characterized in humans, but also exist with a similar etiology in other mammals, and can be treated by administering pharmaceutical compositions of the present invention.

The term “treating” or “treatment” as stated throughout this document is used conventionally, e.g., the management or care of a subject for the purpose of combating, alleviating, reducing, relieving, improving the condition of, etc., of a disease or disorder, such as a carcinoma.

The present invention also provides methods for the treatment of disorders associated with aberrant mitogen extracellular kinase activity, including, but not limited to stroke, heart failure, hepatomegaly, cardiomegaly, diabetes, Alzheimer's disease, cystic fibrosis, symptoms of xenograft rejections, septic shock or asthma.

Effective amounts of compounds of the present invention can be used to treat such disorders, including those diseases (e.g., cancer) mentioned in the Background section above. Nonetheless, such cancers and other diseases can be treated with compounds of the present invention, regardless of the mechanism of action and/or the relationship between the kinase and the disorder.

The phrase “aberrant kinase activity” or “aberrant tyrosine kinase activity,” includes any abnormal expression or activity of the gene encoding the kinase or of the polypeptide it encodes. Examples of such aberrant activity, include, but are not limited to, over-expression of the gene or polypeptide; gene amplification; mutations which produce constitutively-active or hyperactive kinase activity; gene mutations, deletions, substitutions, additions, etc.

The present invention also provides for methods of inhibiting a kinase activity, especially of mitogen extracellular kinase, comprising administering an effective amount of a compound of the present invention, including salts, polymorphs, metabolites, hydrates, solvates thereof, and diastereoisomeric forms thereof. Kinase activity can be inhibited in cells (e.g., in vitro), or in the cells of a mammalian subject, especially a human patient in need of treatment.

### EXPERIMENTAL SECTION

The following Table lists the abbreviations used in this paragraph, and in the Examples section.

| Abbreviation | Meaning |
|---|---|
| BINAP | 2,2′-bis(diphenylphosphino)-1,1′-binaphthyl |
| rac-BINAP | rac-(2,2′-bis(diphenylphosphino)-1,1′-binaphthyl |
| (R)-BINAP | (R)-(2,2′-bis(diphenylphosphino)-1,1′-binaphthyl |
| DMF | N,N-dimethylforamide |
| DMSO | dimethyl sulfoxide |
| h | hour |
| HATU | N-[(dimethylamino)(3H-[1,2,3]triazolo[4,5-b]pyridin-3- |
| | yloxy)methylidene]-N-methylmethanaminium |
| | hexafluorophosphate |
| HPLC, LC | high performance liquid chromatography |
| Hünig base | N-ethyl-N-isopropylpropan-2-amine |
| M | Molar (M = mol/L) |
| min | minute |
| MS | mass spectroscopy |
| NMR | nuclear magnetic resonance |
| NMP | N-methylpyrrolidinone |
| Pd(OAc)₂ | Palladium acetate |
| PdCl₂(PPh₃)₂ | dichlorobis(triphenylphosphine)palladium(II) |
| Pd(dba)₂ | (1E,4E)-1,5-diphenylpenta-1,4-dien-3-one-palladium |
| | (2:1) |
| Pd₂dba₃ | Tris(dibenzylideneacetone)dipalladium(0) |
| Pd(dppf)Cl₂ | dichloro[1,1′- |
| | bis(diphenylphosphino)ferrocene]palladium(II) |
| Pd(dppf)Cl₂ | dichloro[1,1′- |
| CH₂Cl₂ | bis(diphenylphosphino)ferrocene]palladium(II) |
| | dichloromethane adduct |
| Pd-Brett- | chloro[2-(dicyclohexylphosphino)-3,6-dimethoxy-2′-4′-6′- |
| Phos- | tri-iso-propyl-1,1′-biphenyl] [2-(2- |
| pre-cat | aminoethyl)phenyl]palladium(II) |
| Pd-tBu-X- | chloro(2-di-tert-butylphosphino-2′,4′,6′-tri-isopropyl-1,1′- |
| Phos- | biphenyl)[2-(2-aminoethyl)phenyl] palladium(II), |
| pre-cat | |
| Pd-X-Phos- | chloro(2-dicyclohexylphosphino-2′,4′,6′-tri-isopropyl-1,1′- |
| pre-cat | biphenyl)[2-(2-aminoethyl)phenyl] palladium(II) methyl- |
| | tert-butylether adduct |
| PPh₃ | triphenylphosphine |
| P(oTol)₃ | tri-o-tolylphosphine |
| Rac | racemic |
| Rₜ | retention time |
| r.t. | room temperature |
| TBAF | Tetrabutylammoniumfluorid |
| TBTU | N-[(1H-benzotriazol-1- |
| | yloxy)(dimethylamino)methylene]- |
| | N-methylmethanaminium tetrafluoroborate |
| THF | tetrahydrofurane |
| TFA | trifluoroacetic acid |
| X-Phos | 2-dicyclohexylphosphino-2′,4′,6′-triisopropylbiphenyl |

NMR peak forms are stated as they appear in the spectra, possible higher order effects have not been considered.

The schemes and procedures described below illustrate general synthetic routes to the compounds of general formula (I) of the invention and are not intended to be limiting. It is clear to the person skilled in the art that the order of transformations as exemplified in the Schemes can be modified in various ways. The order of transformations exemplified in the Schemes is therefore not intended to be limiting. In addition, interconversion of any of the substituents R¹, R², R^{5a}, R^{5b}, R⁶, R⁷, R⁸ or R⁹ can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent paragraphs.

A first reaction scheme is outlined infra:

### Synthesis of Compounds of General Formula (I) of the Present Invention

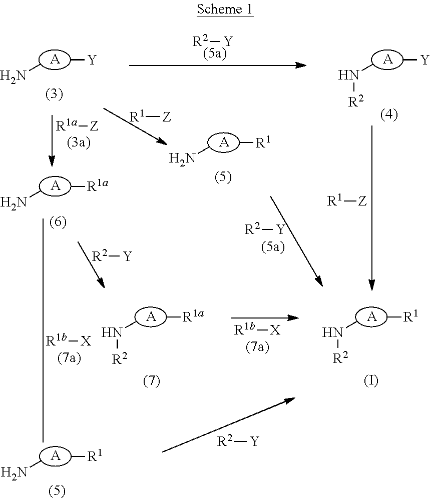

wherein A, R¹, and R² are as defined for the compounds of general formula (I), supra, and Y represents a leaving group, such as a halogen atom or a trifluoromethylsulphonyloxy or nonafluorobutylsulphonyloxy group for example, and Z represents a suitable functional group via which the R¹ of the R¹—Z compound can be coupled, by a coupling reaction, onto the Y-bearing carbon atom of a compound (4), thereby replacing said Y with said R¹ moiety. Many aryl halides of the formula R²—Y may be obtained commercially. Reagents of the general structure R^{1a}—Z and R¹—Z can for example be aryl boronic acids or aryl boronic esters. Many such reagents of the general structures R^{1a}—Z and R¹—Z are also commercially available. Reagents of the general structures R^{1a}—Z and R¹—Z can be prepared from aryl halides [see for example K. L. Billingslay, T. E. Barde, S. L Buchwald, Angew. Chem. 2007, 119, 5455 or T. Graening, Nachrichten aus der Chemie, January 2009, 57, 34].

R^{1a} can be converted to R¹ in one or several steps. Typically, R^{1a} can be a protected phenyl-amine, especially-phenyl-NH-Boc, or a phenyl-carboxylic acid, [-phenyl-C(O)OH] or a-phenyl-carboxylic acid ester [-phenyl-C(O)O-alkyl]. For example, when R^{1a} is a phenyl group to which an —NH₂ substituent is bound, this —NH₂ substituent may be allowed to react with a compound of general formula R^{1b}—X (7a), in which R^{1b} is —C(═O)R⁶ or —C(═O)NR⁶R⁷(R⁶ and R⁷ being as defined as for compounds of general formula (I) of the present invention as defined in the claims), and X is a suitable functional group (e.g. an —OH, —O—C₁-C₆-alkyl group, or a halogen atom), via which the R^{1b} of the R^{1b}—X compound (7a) can be coupled, via a coupling reaction, such as an amide coupling reaction for example, onto the —NH₂ substituent bound to the phenyl group R^{1a} of compound (7), thereby replacing said X with said R^{1a}, thus providing a compound of general formula (I) of the present invention.

Intermediates of general formula (3) can be converted to intermediates of general formula (4) by reaction with suitable aryl compounds R²—Y, preferably aryl bromides, or aryl iodides or for example aryl trifluoromethylsulphonates or aryl nonafluorobutylsulphonates in the presence of a suitable base, such as, for example NaOtBu or caesium carbonate or potassium phosphate, and a suitable catalyst/Ligand system, such as for example Pd₂(dba)₃/rac-BINAP, Pd₂dba₃/X-Phos, Pd₂dba₃/tBu-X-Phos, Pd₂dba₃/Brett-Phos, Pd—X-Phos-pre-cat/X-Phos, Pd-tBu-X-Phos-pre-cat/tBu-X-Phos, Pd-Brett-Phos-pre-cat/Brett-Phos in a suitable solvent such as THF, toluene, xylene, DME, or NMP, or mixtures of these solvents at temperatures ranging from room temperature to the 200° C. The person skilled in the art will recognise that the appropriate choice of reaction conditions, such as temperature, choice of solvent and catalyst system is critical for preferred derivatization at the amino group of intermediates of general formula (3).

Alternatively, intermediates of general formula (3) can be converted to intermediates of general formula (4) by reaction with suitable phenyl or pyridyl compounds R²—Y, preferably phenyl chlorides, and more preferably 2-chloro-pyridines or 6-chloro-pyridines in the presence of a suitable base, such as, for example sodium hydride in a suitable solvent such as THF, DMF, DME, or NMP, preferably THF or NMP or mixtures of these solvents at temperatures ranging from room temperature to the 200° C., preferably 130° C. in a microwave vessel.

Intermediates of general formula (4) can be converted to compounds of general formula (I) by reaction with a suitable reagent, like for example a boronic acid derivative in the presence of a suitable catalyst system, like for example Pd(OAc)₂ and P(oTol)₃, or PdCl₂(PPh₃)₂ and PPh₃ and a suitable base, like for example aqueous potassium carbonate in a suitable solvent, like for example THF, DME, ethanol or 1-propanol or mixtures of these solvents at temperatures ranging from room temperature to 200° C., preferably the boiling point of the used solvent.

In an alternative route for the synthesis of compounds of general formula (I), intermediates of general formula (3) can be reacted with a suitable reagent, like for example a boronic acid derivative in the presence of a suitable catalyst system, like for example Pd(OAc)₂ and P(oTol)₃, or PdCl₂(PPh₃)₂ and PPh₃ and a suitable base, like for example aqueous potassium carbonate in a suitable solvent, like for example THF, DME, ethanol or 1-propanol or mixtures of these solvents at temperatures ranging from room temperature to 200° C., preferably the boiling point of the used solvent to furnish intermediates of the general formula (5).

Intermediates of general formula (5) can be converted to compounds of general formula (I) by reaction with suitable phenyl or pyridyl compounds R²—Y, preferably bromides, iodides, trifluoromethylsulphonates or nonafluorobutylsulphonates in the presence of a suitable base, such as, for example NaOtBu or caesium carbonate or potassium phosphate, and a suitable catalyst/ligand system, such as for example Pd₂(dba)₃/rac-BINAP, Pd₂dba₃/X-Phos, Pd₂dba₃/tBu-X-Phos, Pd₂dba₃/Brett-Phos, Pd—X-Phos-pre-cat/X-Phos, Pd-tBu-X-Phos-pre-cat/tBu-X-Phos, Pd-Brett-Phos-pre-cat/Brett-Phos in a suitable solvent such as THF, toluene, xylene, DME, or NMP, or mixtures of these solvents at temperatures ranging from room temperature to 200° C.

Also as depicted in Scheme 1, is a further alternative route for the synthesis of compounds of general formula (I): Intermediates of general formula (3) can be converted to intermediates of general formula (6) by a coupling reaction as described supra for synthesis of intermediate of general formula (5), thereby replacing said Y of intermediates of general formula (3) with said R^{1a} moiety.

Intermediates of general formula (6) can then be converted to intermediates of general formula (7) by a coupling reaction as described supra for synthesis of intermediates of general formula (4), thereby forming a bond between NH and said R² moiety. Intermediates of general formula (7) can then be converted to compounds of general formula (I) by one or more further transformations. These can be modifications such as cleavage of protecting groups, reduction or oxidation of functional groups, halogenation, metallation, substitution or other reactions known to the person skilled in the art, for example the formation of an amide bond, the formation of a urea, or the formation of a sulfonamide, thereby converting R^{1a} to said R¹ moiety.

Additionally, intermediates of general formula (6) can be converted to intermediates of general formula (5) by one or more further transformations. These can be modifications such as cleavage of protecting groups, reduction or oxidation of functional groups, halogenation, metallation, substitution or other reactions known to the person skilled in the art, for example the formation of an amide bond, the formation of a urea, or the formation of a sulphonamide, thereby converting R^{1a} to said R¹ moiety.

Intermediates of general formula (5) can then be converted to compounds of general formula (I) by a coupling reaction as described supra for synthesis of intermediates of general formula (4), thereby forming a bond between NH and said R² moiety.

The compounds and intermediates produced according to the methods of the invention may require purification. Purification of organic compounds is well known to the person skilled in the art and there may be several ways of purifying the same compound. In some cases, no purification may be necessary. In some cases, the compounds may be purified by crystallisation. In some cases, impurities may be stirred out using a suitable solvent. In some cases, the compounds may be purified by chromatography, particularly flash chromatography, using for example pre-packed silica gel cartridges, e.g. from Separtis such as Isolute® Flash silica gel (silica gel chromatography) or Isolute® Flash NH2 silica gel (aminophase-silica-gel chromatography) in combination with a suitable chromatographic system such as a Flashmaster II (Separtis) or an Isolera system (Biotage) and eluents such as, for example, gradients of hexane/ethyl acetate or DCM/methanol. In some cases, the compounds may be purified by preparative HPLC using, for example, a Waters autopurifier equipped with a diode array detector and/or on-line electrospray ionisation mass spectrometer in combination with a suitable pre-packed reverse phase column and eluants such as, for example, gradients of water and acetonitrile which may contain additives such as trifluoroacetic acid, formic acid or aqueous ammonia.

Names of compounds were generated using ACD/Name Batch ver. 12.00 or ACD/Name Batch ver. 12.01. Names of compounds in table format were generated using ACD/Name Batch ver. 12.00.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown.

Unless specified otherwise, suffixes to chemical names or structural formulae such as “hydrochloride”, “trifluoroacetate”, “sodium salt”, or “x HCl”, “x CF₃COOH”, “x Na⁺”, for example, are to be understood as not a stoichiometric specification, but solely as a salt form.

This applies analogously to cases in which synthesis intermediates or example compounds or salts thereof have been obtained, by the preparation and/or purification processes described, as solvates, such as hydrates with (if defined) unknown stoichiometric composition.

### HPLC Methods:

### Method 1:

Instrument: Waters Acquity UPLCMS ZQ4000; Column: Acquity UPLC BEH C18 1.7 μm, 50×2.1 mm; eluent A: water+0.05 vol % formic acid, Eluent B: acetonitrite+0.05 vol % formic acid gradient: 0-1.6 min 1-99% B, 1.6-2.0 min 99% B; flow 0.8 mL/min; temperature: 60° C.; injection: 2 μL; DAD scan: 210-400 nm; ELSD.

### Method 2:

Instrument: Waters Acquity UPLCMS SQD 3001; Column: Acquity UPLC BEH C18 1.7 μm, 50×2.1 mm; eluent A: water+0.1 vol % formic acid (95%), eluent B: acetonitrile, gradient: 0-1.6 min 1-99% B, 1.6-2.0 min 99% B; flow 0.8 mL/min; temperature: 60° C.; injection: 2 μL; DAD scan: 210-400 nm; ELSD.

### Method 3:

Instrument: Waters Acquity UPLCMS SQD; Column: Acquity UPLC BEH C18 1.7 μm, 50×2.1 mm; eluent A: water+0.05 vol % formic acid (95%), eluent B: acetonitrile+0.05 vol % formic acid (95%), gradient: 0-1.6 min 1-99% B, 1.6-2.0 min 99% B; flow 0.8 mL/min; temperature: 60° C.; injection: 2 μL; DAD scan: 210-400 nm; ELSD.

### Method 4:

Instrument: Waters Acquity UPLC-MS SQD; Column: Acquity UPLC BEH C18 1.7 50×2.1 mm; eluent A: water+0.1 vol % formic acid (99%), eluent B: acetonitrile; gradient: 0-1.6 min 1-99% B, 1.6-2.0 min 99% B; flow 0.8 mL/min; temperature: 60° C.; injection: 2 μL; DAD scan: 210-400 nm; ELSD.

### Method 5:

Instrument: Waters Acquity UPLCMS SQD 3001; Column: Acquity UPLC BEH C18 1.7 μm, 50×2.1 mm; eluent A: water+0.2 vol. % ammonia (32%), eluent B: acetonitrile, gradient: 0-1.6 min 1-99% B, 1.6-2.0 min 99% B; flow 0.8 mL/min; temperature: 60° C.; injection: 2 μL; DAD scan: 210-400 nm; ELSD.

### Method 6

Instrument: Waters Acquity UPLC-MS SQD; Column: Acquity UPLC BEH C18 1.7 50×2.1 mm; eluent A: water+0.2% vol. ammonia (32%), eluent B: acetonitrile; gradient: 0-1.6 min 1-99% B, 1.6-2.0 min 99% B; flow 0.8 mL/min; temperature: 60° C.; injection: 2 μl; DAD scan: 210-400 nm; ELSD.

### Method 7

Instrument: Waters Acquity UPLC-MS ZQ; column: Acquity UPLC BEH C18 1.7 50×2.1 mm; eluent A: water+0.1% vol. formic acid (99%), eluent B: acetonitrile; gradient: 0-1.6 min 1-99% B, 1.6-2.0 min 99% B; flow 0.8 mL/min; temperature: 60° C.; injection: 2 μl; DAD scan: 210-400 nm; ELSD.

### Method 8:

Instrument: Waters Acquity UPLCMS SQD; Column: Acquity UPLC BEH C18 1.7 μm, 50×2.1 mm; Eluent A: water+0.2% vol. ammonia (32%), eluent B: acetonitrile; gradient: 0-1.6 min 1-99% B, 1.6-2.0 min 99% B; flow 0.8 mL/min; temperature: 60° C.; injection: 2 μl; DAD scan: 210-400 nm; ELSD.

### Intermediates

### Intermediate Example 01.01

### ethyl [(6-bromopyridazin-3-yl)carbamothioyl]carbamate

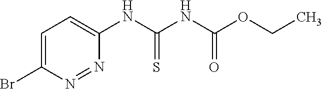 [SMILES: CCOC(=O)NC(=S)Nc1ccc(Br)nn1]

Ethoxycarbonyl isothiocyanate (9.12 g) was added to a stirred solution of 6-bromopyridazin-3-amine (11 g) in dioxane (113 mL). The mixture was stirred for 16 h at r.t. A white solid precipitated. Hexane (110 mL) was added and the white solid was collected by filtration to give 16.6 g of the title compound.

### Intermediate Example 01.02

### 6-bromo[1,2,4]triazolo[1,5-b]pyridazin-2-amine

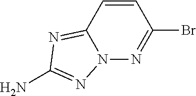 [SMILES: Nc1nc2ccc(Br)nn2n1]

Hydroxylammonium chloride (13.7 g) was suspended in methanol (70 mL), and ethanol (70 mL) and Hünig Base (20.5 mL) were added at r.t. The mixture was heated to 60° C., ethyl [(6-bromopyridazin-3-yl)carbamothioyl]carbamate (10.0 g) was added portionwise, and the mixture was stirred at 60° C. for 2 h. A solid precipitated and was collected by filtration. The solid was stirred with aqueous sodium hydroxide (100 mL, c=1M) for 1 h. The solid was collected by filtration and was washed with water and dried in vacuum to give 5.1 g of the title compound.

### Intermediate Example 01.03

### tert-butyl [4-(2-amino[1,2,4]triazolo[1,5-b]pyridazin-6-yl)phenyl]carbamate

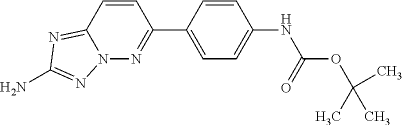 [SMILES: CC(C)(C)OC(=O)Nc1ccc(-c2ccc3nc(N)nn3n2)cc1]

To a stirred solution of 6-bromo[1,2,4]triazolo[1,5-b]pyridazin-2-amine (5.0 g) in 1-propanol (135 mL) was added 2M potassium carbonate solution (35 mL), {4-[(tert-butoxycarbonyl) amino]phenyl}boronic acid (6.1 g), triphenylphosphine (306 mg) and PdCl₂(PPh₃)₂(953 mg). The mixture was heated to reflux for 1 h. Further triphenylphosphine (306 mg) and PdCl₂(PPh₃)₂ (953 mg) were added and the mixture was heated to reflux for 1 h. The mixture was stirred at room temperature for 16 h, a solid precipitated and was collected by filtration. The solid was stirred with water (100 mL) for 1 h. The solid was collected by filtration and dried in vacuum to give 5.6 g of the title compound.

### Intermediate Example 01.04

### 6-(4-aminophenyl)[1,2,4]triazolo[1,5-b]pyridazin-2-amine

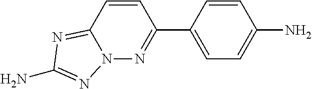 [SMILES: Nc1ccc(-c2ccc3nc(N)nn3n2)cc1]

To a stirred suspension of tert-butyl [4-(2-amino[1,2,4]triazolo[1,5-b]pyridazin-6-yl)phenyl]carbamate (5.6 g) in dichloromethane (56 mL) was added TFA (13.2 mL). The mixture was stirred at r.t. for 70 h. The mixture was concentrated in vacuum. Water was added and the solution was filtered. An aqueous solution of sodium hydroxide was added until pH 11 was reached. A solid precipitated and was collected by filtration and dried in vacuum to give 2.7 g of the title compound.

### Intermediate Example 01.05

### N-[4-(2-amino[1,2,4]triazolo[1,5-b]pyridazin-6-yl)phenyl]-2-(4-fluorophenyl)acetamide

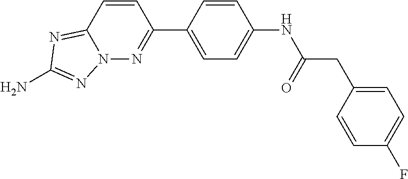 [SMILES: Nc1nc2ccc(-c3ccc(NC(=O)Cc4ccc(F)cc4)cc3)nn2n1]

To a stirred solution of 6-(4-aminophenyl)[1,2,4]triazolo[1,5-b]pyridazin-2-amine (2.70 g) in THF (135 mL) was added Hünig Base (2.29 mL), (4-fluorophenyl)acetic acid (2.02 g), and HATU (4.99 g). The mixture was stirred at room temperature for 24 h. Water was added and the mixture was stirred at room temperature for 1 h. The precipitated solid was collected by filtration, was washed with ethanol and hexane and was dried in vacuum to give 2.4 g of the title compound.

### Intermediate Example 02.01

### ethyl [(5-bromopyrazin-2-yl)carbamothioyl]carbamate

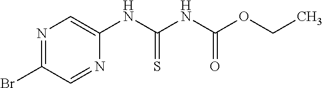 [SMILES: CCOC(=O)NC(=S)Nc1cnc(Br)cn1]

Ethoxycarbonyl isothiocyanate (49.7 g) was added to a stirred solution of 5-bromopyrazin-2-amine (60.0 g) in dioxane (600 mL). The mixture was stirred for 48 h at r.t. A white solid precipitated. The white solid was collected by filtration to give 78.5 g of the title compound.

### Intermediate Example 02.02

### 6-bromo[1,2,4]triazolo[1,5-a]pyrazin-2-amine

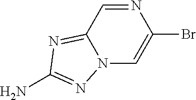 [SMILES: Nc1nc2cnc(Br)cn2n1]

Hydroxylammonium chloride (99.1 g) was suspended in methanol (498 mL), and ethanol (450 mL) and Hünig Base (150 mL) were added at r.t. The mixture was heated to 60° C., ethyl [(5-bromopyrazin-2-yl)carbamothioyl]carbamate (75 g) was added portionwise, and the mixture was stirred at 60° C. for 2 h. Hexane (500 mL) was added, and a solid was collected by filtration. The solid was stirred with water (75 mL) for 1 h. The solid was collected by filtration and was washed with water and dried in vacuum to give 46.2 g of the title compound.

### Intermediate Example 02.03

### tert-butyl [4-(2-amino[1,2,4]triazolo[1,5-a]pyrazin-6-yl)phenyl]carbamate

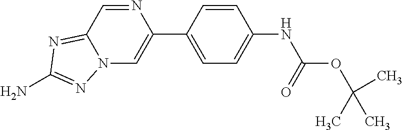 [SMILES: CC(C)(C)OC(=O)Nc1ccc(-c2cn3nc(N)nc3cn2)cc1]

To a stirred solution of 6-bromo[1,2,4]triazolo[1,5-a]pyrazin-2-amine (10.0 g) in 1-propanol (420 mL) was added 2M potassium carbonate solution (70 mL), {4-[(tert-butoxycarbonyl) amino]phenyl} boronic acid (15.6 g), triphenylphosphine (613 mg) and PdCl₂(PPh₃)₂(3.28 g). The mixture was heated to reflux for 2 h. Water was added and the mixture was stirred at room temperature for 15 minutes. A solid precipitated and was collected by filtration and dried in vacuum to give 14.7 g of the title compound.

### Intermediate Example 02.04

### 6-(4-aminophenyl)[1,2,4]triazolo[1,5-a]pyrazin-2-amine

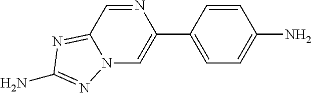 [SMILES: Nc1ccc(-c2cn3nc(N)nc3cn2)cc1]

To a stirred suspension of tert-butyl [4-(2-amino[1,2,4]triazolo[1,5-a]pyrazin-6-yl)phenyl]carbamate (14.7 g) in dichloromethane (115 mL) was added TFA (52 mL). The mixture was stirred at r.t. for 5 h. The mixture was concentrated in vacuum to approx. 40 mL. Water was added and an aqueous solution of potassium carbonate was added until pH 11 was reached. A solid precipitated and was collected by filtration and dried in vacuum to give 8.7 g of the title compound.

### Intermediate Example 02.05

### N-[4-(2-amino[1,2,4]triazolo[1,5-a]pyrazin-6-yl)phenyl]-2-(4-fluorophenyl)acetamide

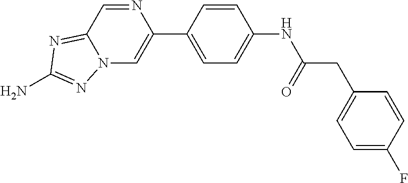 [SMILES: Nc1nc2cnc(-c3ccc(NC(=O)Cc4ccc(F)cc4)cc3)cn2n1]

To a stirred solution of 6-(4-aminophenyl)[1,2,4]triazolo[1,5-a]pyrazin-2-amine (4.00 g) in THF (270 mL) was added Hünig Base (5.4 mL), (4-fluorophenyl)acetic acid (3.48 g), and HATU (12.1 g). The mixture was stirred at room temperature for 24 h. Water was added and the mixture was stirred at room temperature for 16 h. The precipitated solid was collected by filtration, was washed with methanol and ether and was dried in vacuum to give 5.4 g of the title compound.

### Intermediate Example 02.06

### N-[4-(2-amino[1,2,4]triazolo[1,5-a]pyrazin-6-yl)phenyl]-2-phenylacetamide

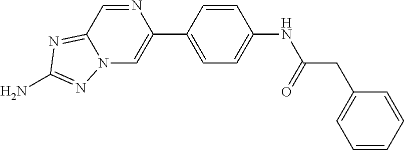 [SMILES: Nc1nc2cnc(-c3ccc(NC(=O)Cc4ccccc4)cc3)cn2n1]

To a stirred suspension of 6-(4-aminophenyl)[1,2,4]triazolo[1,5-a]pyrazin-2-amine (300 mg) in DMF (10 mL) was added potassium carbonate (0.49 g), phenylacetic acid (199 mg), and HATU (554 mg). The mixture was stirred at room temperature for 24 h. Water was added and the mixture was stirred at room temperature for 1 h. The mixture was extracted with ethyl acetate. The organic phase was dried (sodium sulfate) and the solvent was removed in vacuum to give a solid that was recrystallized from ethanol to give 330 mg of the title compound.

### Intermediate Example 02.07

### N-[4-(2-amino[1,2,4]triazolo[1,5-a]pyrazin-6-yl)phenyl]-2-(3,4-difluorophenyl)acetamide

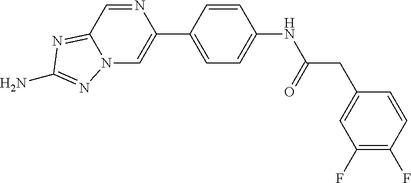 [SMILES: Nc1nc2cnc(-c3ccc(NC(=O)Cc4ccc(F)c(F)c4)cc3)cn2n1]

To a stirred solution of 6-(4-aminophenyl)[1,2,4]triazolo[1,5-a]pyrazin-2-amine (300 mg) in THF (25 mL) was added Hünig Base (0.25 mL), (3,4-difluorophenyl)acetic acid (256 mg), and HATU (555 mg). The mixture was stirred at room temperature for 16 h. Water was added and the mixture was stirred at room temperature for 1 h. The precipitated solid was collected by filtration and was washed with ethanol and ether. The solid was recrystallized from ethanol to give 500 mg of the title compound.

### Intermediate Example 02.08

### tert-butyl(4-{2-[(2-cyanophenyl)amino][1,2,4]triazolo[1,5-a]pyrazin-6-yl}phenyl)carbamate

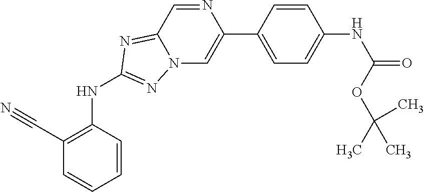 [SMILES: CC(C)(C)OC(=O)Nc1ccc(-c2cn3nc(Nc4ccccc4C#N)nc3cn2)cc1]

To a stirred suspension of tert-butyl [4-(2-amino[1,2,4]triazolo[1,5-a]pyrazin-6-yl)phenyl]carbamate (2.0 g) in toluene (10 mL) and NMP (0.4 mL) was added 2-bromobenzonitrile (1.57 g), rac-BINAP (389 mg) and Pd₂dba₃ (281 mg) and cesium carbonate (6.1 g) and the flask was degassed twice and backfilled with argon. The mixture was heated to reflux for 16 h. Water was added and the reaction mixture was extracted with ethyl acetate. The organic phase was washed with saturated sodium chloride solution, dried (sodium sulfate) and the solvent was removed in vacuum to give a solid that was triturated with dichloromethane to give 1.3 g of the title compound.

### Intermediate Example 02.09

### 4-(2-amino[1,2,4]triazolo[1,5-a]pyrazin-6-yl)-2,6-dimethylphenol

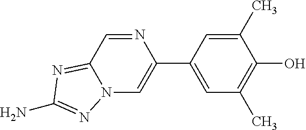 [SMILES: Cc1cc(-c2cn3nc(N)nc3cn2)cc(C)c1O]

To a solution of 2,6-dimethyl-4-(4,4,5,5-tetramethyl-1,3,2-dioxaborolan-2-yl)phenol (777 mg) in dichloromethane was added aqueous hydrochloric acid (c=2N, 7.5 mL). The mixture was vigorously shaken for 5 minutes, the organic phase was separated and the aqueous phase was extracted with a mixture of dichloromethane and methanol (100:1). The combined organic phases were dried (sodium sulfate) and the solvent was removed in vacuum.

The residue (720 mg) was dissolved in 1-propanol (35 mL) and a 2 M potassium carbonate solution (3.5 mL), 6-bromo[1,2,4]triazolo[1,5-a]pyrazin-2-amine (500 mg), triphenylphosphine (13 mg) and PdCl₂(PPh₃)₂(164 mg) were added. The mixture was heated to reflux for 3 h, water (100 mL) was added and the mixture was extracted with a mixture of ethyl acetate and hexane (3:1). The organic phase was washed with water and with saturated sodium chloride solution, dried (sodium sulfate) and the solvent was removed in vacuum. Silicagel chromatography gave a solid that was triturated with ethanol to give 250 mg of the title compound.

### Intermediate Example 02.10

### N-[4-(2-amino[1,2,4]triazolo[1,5-a]pyrazin-6-yl)phenyl]-2-cyclopropylacetamide

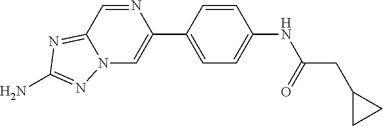 [SMILES: Nc1nc2cnc(-c3ccc(NC(=O)CC4CC4)cc3)cn2n1]

To a stirred solution of 6-(4-aminophenyl)[1,2,4]triazolo[1,5-a]pyrazin-2-amine (320 mg) in THF (27 mL) was added Hünig Base (0.27 mL), cyclopropylacetic acid (156 mg), and HATU (592 mg). The mixture was stirred at room temperature for 64 h. Water was added and the mixture was stirred at room temperature for 1 h. The precipitated solid was collected by filtration and was washed with ethanol and ether to give 420 mg of the title compound.

### Intermediate Example 02.11

### 3-(2-amino[1,2,4]triazolo[1,5-a]pyrazin-6-yl)benzoic acid

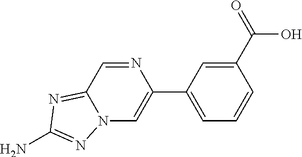 [SMILES: Nc1nc2cnc(-c3cccc(C(=O)O)c3)cn2n1]

To a stirred solution of 6-bromo[1,2,4]triazolo[1,5-a]pyrazin-2-amine (5.0 g) in 1-propanol (350 mL) was added 2M potassium carbonate solution (35 mL), 3-(dihydroxyboryl)benzoic acid (5.04 g), triphenylphosphine (306 mg) and PdCl₂(PPh₃)₂(1.64 g). The mixture was heated to reflux for 2 h. The solvent was removed in vacuum. An aqueous solution of citric acid (10% w/w) was added and the mixture was extracted with ethyl acetate. The organic phase was separated, filtered and the solvent was removed in vacuum to give 5.82 g of the title compound.

### Intermediate Example 02.12

### ethyl 3-(2-amino[1,2,4]triazolo[1,5-a]pyrazin-6-yl)benzoate

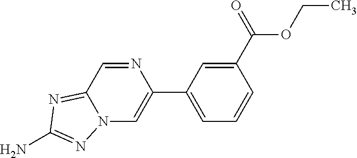 [SMILES: CCOC(=O)c1cccc(-c2cn3nc(N)nc3cn2)c1]

To a stirred suspension of 3-(2-amino[1,2,4]triazolo[1,5-a]pyrazin-6-yl)benzoic acid (6.0 g) in ethanol (120 mL) was added thionyl dichloride (15.4 mL) with ice bath cooling. The mixture was heated to reflux for 48 h. Further ethanol (100 mL) and further thionyl dichloride (15.4 mL) was added with ice bath cooling and the mixture was heated to reflux for further 64 h. The mixture was cooled to room temperature and a solid was collected by filtration. The solid was dissolved in a mixture of dichloromethane and methanol (10:1) and was washed with an aqueous solution of sodium bicarbonate. The organic phase was separated, filtered and the solvent was removed in vacuum to give 4.31 g of the title compound.

### Intermediate Example 02.13

### ethyl 3-{2-[(2-cyanophenyl)amino][1,2,4]triazolo[1,5-a]pyrazin-6-yl}benzoate

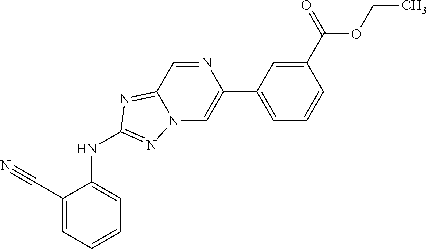 [SMILES: CCOC(=O)c1cccc(-c2cn3nc(Nc4ccccc4C#N)nc3cn2)c1]

To a stirred suspension of ethyl 3-(2-amino[1,2,4]triazolo[1,5-a]pyrazin-6-yl)benzoate (500 mg) in toluene (15 mL) and NMP (0.3 mL) was added 2-bromobenzonitrile (662 mg), rac-BINAP (112 mg) and Pd₂dba₃ (81 mg) and cesium carbonate (1.76 g) and the flask was degassed twice and backfilled with argon. The mixture was heated to reflux for 3 h. Water was added and the reaction mixture was extracted with ethyl acetate. The organic phase was washed with saturated sodium chloride solution, dried (sodium sulfate) and the solvent was removed in vacuum. Silicagel chromatography gave 548 mg of the title compound.

### Intermediate Example 02.14

### 3-{2-[(2-cyanophenyl)amino][1,2,4]triazolo[1,5-a]pyrazin-6-yl}benzoic acid

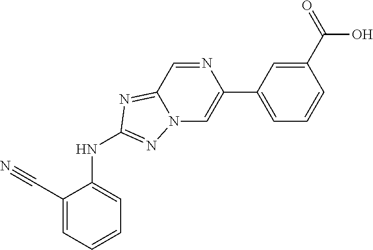 [SMILES: N#Cc1ccccc1Nc1nc2cnc(-c3cccc(C(=O)O)c3)cn2n1]

To a stirred solution of ethyl 3-{2-[(2-cyanophenyl)amino][1,2,4]triazolo[1,5-a]pyrazin-6-yl}benzoate (444 mg) in methanol (14 mL) and tetrahydrofurane (7.0 mL) was added an aqueous solution of sodium hydroxide (11.6 mL, c=2.5 M). The mixture was stirred at room temperature for 2 h. An aqueous solution of hydrochloric acid (c=2 N) was added until pH 3 was reached. The mixture was stirred for 10 minutes and the precipitated solid was collected by filtration to give 407 mg of the title compound.

### Intermediate Example 03.01

### tert-butyl [4-(2-amino-1,3-benzothiazol-6-yl)phenyl]carbamate

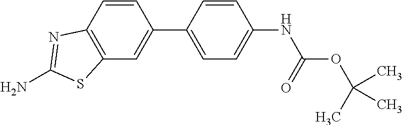 [SMILES: CC(C)(C)OC(=O)Nc1ccc(-c2ccc3nc(N)sc3c2)cc1]

To a stirred solution of 6-bromo-1,3-benzothiazol-2-amine (2.0 g) in 1-propanol (50 mL) was added 2M potassium carbonate solution (13 mL), {4-[(tert-butoxycarbonyl)amino]phenyl}boronic acid (2.28 g), triphenylphosphine (343 mg) and PdCl₂(PPh₃)₂(919 mg). The mixture was heated to reflux for 3 h. The solvent was removed in vacuum, water was added and the mixture was extracted with ethyl acetate. The organic phase was washed with saturated sodium chloride solution, dried (sodium sulfate), filtered through celite and the solvent was removed in vacuum. The residue was triturated with dichloromethane to give 1.21 g of the title compound.

### Intermediate Example 03.02

### 6-(4-aminophenyl)-1,3-benzothiazol-2-amine

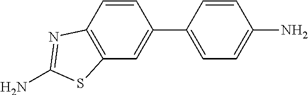 [SMILES: Nc1ccc(-c2ccc3nc(N)sc3c2)cc1]

To a stirred solution of tert-butyl [4-(2-amino-1,3-benzothiazol-6-yl)phenyl]carbamate (1.2 g) in dichloromethane (6.0 mL) was added TFA (2.7 mL). The mixture was stirred at room temperature for 3 h. A saturated solution of potassium carbonate was added until pH 9 was reached. The mixture was extracted with dichloromethane. The solution was dried (sodium sulfate) and the solvent was removed in vacuum. Aminophase-silica-gel chromatography gave a solid that was triturated with dichloromethane to give 662 mg of the title compound.

### Intermediate Example 04.01

### methyl 4-bromo-3-methoxybenzoate

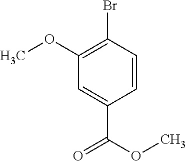

To a stirred solution of methyl 4-bromo-3-hydroxybenzoate (10.0 g) in DMF (50 mL) was added potassium carbonate (17.9 g) and iodomethane (9.2 mg). The mixture was stirred at room temperature for 2 h. Ethyl acetate was added and the mixture was washed with water. The organic phase was washed with saturated sodium chloride solution, dried (sodium sulfate) and the solvent was removed in vacuum to give 10 g of the title compound, that was used without further purification.

¹H-NMR (400 MHz, DMSO-d₆): δ [ppm]=3.82 (s, 3H), 3.87 (s, 3H), 7.41 (dd, 1H), 7.47 (d, 1H), 7.67 (d, 1H).

### Intermediate Example 04.02

### 4-bromo-3-methoxybenzoic acid

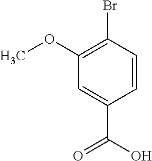

To a stirred solution of methyl 4-bromo-3-methoxybenzoate (11.2 g) in THF (130 mL), methanol (45 mL) and water (45 mL) was added a 1 M solution of lithium hydroxide in water (140 mL). The mixture was stirred at room temperature for 1 h. The solvent was removed in vacuum. Water was added and 1 N hydrochloric acid was added with ice bath cooling until pH 4 was reached. The precipitated solid was collected by filtration, washed with water and dried in vacuum to give 10.1 g of the title compound, that was used without further purification.

¹H-NMR (300 MHz, DMSO-d₆): δ [ppm]=3.87 (s, 3H), 7.42 (dd, 1H), 7.50 (d, 1H), 7.68 (d, 1H), 13.21 (br. s., 1H).

### Intermediate Example 04.03

### (4-bromo-3-methoxyphenyl)(morpholin-4-yl)methanone

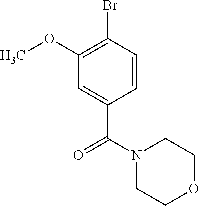 [SMILES: COc1cc(C(=O)N2CCOCC2)ccc1Br]

To a stirred solution of 4-bromo-3-methoxybenzoic acid (3.0 g) in dichloromethane (32 mL) and DMF (1.0 mL) was added oxalyl chloride (1.78 g) at 0° C. The mixture was stirred at room temperature for 1 h. The solvent was removed in vacuum. The residue was dissolved in THF (62 mL) and Hünig Base (6.6 mL) and morpholine (1.66 g) were added. The mixture was stirred at room temperature for 1 h. A half-saturated solution of sodium bicarbonate was added and the mixture was extracted with ethyl acetate. The organic phase was washed with saturated sodium chloride solution, dried (sodium sulfate) and the solvent was removed in vacuum. Silica gel chromatography gave 3.76 g of the title compound.

¹H-NMR (400 MHz, CHLOROFORM-d): δ [ppm]=3.74 (br. s., 8H), 3.92 (s, 3H), 6.83 (dd, 1H), 6.98 (d, 1H), 7.56 (d, 1H).

### Intermediate Example 04.04

### azetidin-1-yl(4-bromo-3-methoxyphenyl)methanone

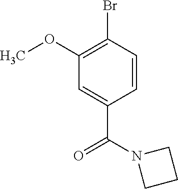

To a stirred solution of 4-bromo-3-methoxybenzoic acid (400 mg) in DMF (4.0 mL) was added potassium carbonate (720 mg), azetidine (148 mg) and TBTU (890 mg). The mixture was stirred at room temperature for 60 h. Water was added, the mixture was stirred for 15 minutes and the solvent was removed in vacuum. Water was added and the mixture was extracted with ethyl acetate. The organic phase was washed with saturated sodium chloride solution, dried (sodium sulfate) and the solvent was removed in vacuum. Silica gel chromatography gave 370 mg of the title compound.

¹H-NMR (400 MHz, DMSO-d₆): δ [ppm]=2.15-2.27 (m, 2H), 3.85 (s, 3H), 4.00 (t, 2H), 4.26 (t, 2H), 7.07 (dd, 1H), 7.21 (d, 1H), 7.61 (d, 1H).

### Intermediate Example 04.05

### (4-Bromo-3-methoxyphenyl)(3-fluoroazetidin-1-yl)methanone

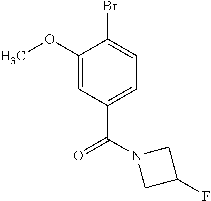 [SMILES: COc1cc(C(=O)N2CC(F)C2)ccc1Br]

To a stirred solution of 4-bromo-3-methoxybenzoic acid (1.4 g) in DMF (15 mL) was added potassium carbonate (2.51 g), 3-fluoroazetidine hydrochloride (1.01 g) and HATU (3.69 g). The mixture was stirred at room temperature for 18 h. Water was added, the mixture was stirred for 15 minutes and the solvent was removed in vacuum. Water was added and the mixture was extracted with ethyl acetate. The organic phase was washed with water, saturated sodium chloride solution, dried (sodium sulfate) and the solvent was removed in vacuum, to give 1.25 g of the title compound.

¹H-NMR (400 MHz, DMSO-d₆): δ [ppm]=3.90 (s, 3H), 3.99-4.16 (m, 1H), 4.31-4.65 (m, 3H), 5.36 (tt, 0.5H), 5.50 (tt, 0.5H), 7.14 (dd, 1H), 7.26 (d, 1H), 7.66 (d, 1H).

### Intermediate Example 05.01

### 2-(4-bromo-3-methoxyphenyl)propan-2-ol

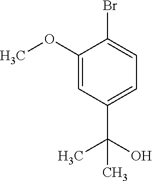

To a stirred solution of methyl 4-bromo-3-methoxybenzoate (5.3 g) in THF (250 mL) was added methyl magnesium bromide (21.5 mL; c=3.0 M) at room temperature and the mixture was heated to reflux for 1 h. A half-saturated aqueous solution of ammonium chloride was added and the mixture was extracted with ethyl acetate. The organic phase was washed with saturated sodium chloride solution, dried (sodium sulfate) and the solvent was removed in vacuum. Silicagel chromatography gave 3.09 g of the title compound.

### Intermediate Example 06.01

### 1-bromo-2-methoxy-4-(methylsulfanyl)benzene

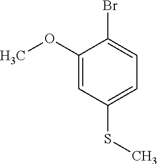

To a stirred solution of 1-bromo-4-fluoro-2-methoxybenzene (4.0 mg) in DMF (40 mL) was added sodium methanethiolate (2.76 g). The mixture was stirred at room temperature for 30 minutes and at 85° C. for 2 h. Water was added and the mixture was extracted with ethyl acetate. The organic phase was washed with saturated sodium chloride solution, dried (sodium sulfate) and the solvent was removed in vacuum. Silica gel chromatography gave 280 mg of the title compound.

¹H-NMR (400 MHz, DMSO-d₆): δ [ppm]=2.46 (s, 3H), 3.82 (s, 3H), 6.74 (dd, 1H), 6.91 (d, 1H), 7.44 (d, 1H).

### 1-bromo-2-methoxy-4-(methylsulfanyl)benzene

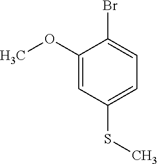

To a stirred solution of 1-bromo-4-fluoro-2-methoxybenzene (10.0 g) in DMF (100 mL) was added sodium methanethiolate (4.44 g). The mixture was stirred at 65° C. for 2 h. The mixture was cooled to 0° C. and methyl iodide (4.55 mL) was added. The mixture was stirred at room temperature for 1 h and further sodium methanethiolate (4.44 g) was added. The mixture was stirred at 65° C. for 1 h. The mixture was cooled to 0° C. and methyl iodide (4.55 mL) was added. The mixture was stirred at room temperature for 1 h. Water was added and the mixture was extracted with ethyl acetate. The organic phase was washed with saturated sodium chloride solution, dried (sodium sulfate) and the solvent was removed in vacuum. Silica gel chromatography gave 6.2 g of the title compound as a 2:1 mixture with the starting material. The mixture was used for the next step without purification.

### Intermediate Example 06.02

### 1-bromo-2-methoxy-4-(methylsulfonyl)benzene

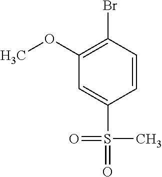

To a stirred solution of 1-bromo-2-methoxy-4-(methylsulfanyl)benzene (265 mg) in chloroform (10 mL) was added 3-chlorobenzenecarboperoxoic acid (mCPBA) (890 mg). The mixture was stirred at room temperature for 1 h. A half-saturated solution of sodium bicarbonate was added and the mixture was extracted with dichloromethane. The organic phase was washed with saturated sodium chloride solution, dried (sodium sulfate) and the solvent was removed in vacuum. Silica gel chromatography gave 252 mg of the title compound.

¹H-NMR (300 MHz, DMSO-d₆): δ [ppm]=3.22 (s, 3H), 3.93 (s, 3H), 7.39 (dd, 1H), 7.50 (d, 1H), 7.84 (d, 1H).

### Intermediate Example 07.01

### 1-(4-Bromo-3-methoxyphenyl)piperazine

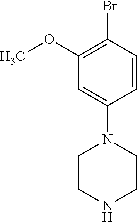

1-(3-Methoxyphenyl)piperazine dihydrochloride (11.97 g, 45.1 mmol) and sodium acetate (4.07 g, 49.7 mmol) were added to a mixture of water (77 mL) and glacial acetic acid (360 mL) at 5° C. Bromine (7.93 g, 49.7 mmol) was added slowly and the mixture was stirred at 0° C. for 1 h. Subsequently, the solvents were removed in vacuo. This residue was dissolved in ethyl acetate and washed with 1N sodium hydroxide solution. The organic layer was dried (sodium sulphate) and the solvent was evaporated. HPLC separation gave 4.39 g of the title compound.

¹H-NMR (400 MHz, DMSO-d6): δ [ppm]=2.79-2.83 (4H), 3.03-3.08 (4H), 3.33 (1H), 3.81 (3H), 6.42 (1H), 6.59 (1H), 7.30 (1H).

### Intermediate Example 07.02

### 1-(4-Bromo-3-methoxyphenyl)-4-methylpiperazine

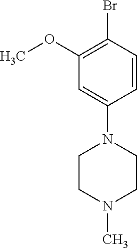 [SMILES: COc1cc(N2CCN(C)CC2)ccc1Br]

To a stirred solution of 1-(4-Bromo-3-methoxyphenyl)piperazine (1.0 g, 3.69 mmol) in methanol (60 mL) were added acetic acid (0.42 mL) and after 5 min sodium cyanoborohydride (463 mg, 7.38 mmol). After additional 5 min formaldehyde solution (33% in water; 0.59 mL, 7.38 mmol) was added. The reaction mixture was stirred at 60° C. for 16 h. Subsequently, the solvents were removed in vacuo. This residue was dissolved in ethyl acetate and washed with 1N sodium hydroxide solution. The organic layer was dried (sodium sulphate) and the solvent was evaporated. Crystallization from pentanes/tert-butyl methyl ether gave 961 mg (91%) of the title compound.

¹H-NMR (400 MHz, DMSO-d₆): δ [ppm]=2.21 (3H), 2.41-2.46 (4H), 3.12-3.17 (4H), 3.81 (3H), 6.44 (1H), 6.61 (1H), 7.30 (1H).

### Intermediate Example 08.01

### Rac-methyl 2-(4-fluorophenyl)propanoate

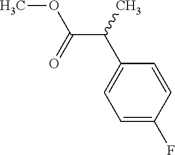

To a stirred solution of diisopropylamine (13.0 g) in tetrahydrofurane (160 mL) was added a solution of n-butyllithium in hexane (51.4 mL; c=2.5 M) at −78° C. The solution was stirred at 0° C. for 15 minutes. The solution was cooled to −78° C. and a solution of methyl (4-fluorophenyl)acetate (18.0 g), dissolved in tetrahydrofurane (40 mL) was added. The solution was stirred at −78° C. for 30 minutes. Methyl iodide (10.0 mL) was added at −78° C., and the solution was allowed to warm up to 0° C. within 1 h. Water was added and the reaction mixture was extracted with ethyl acetate. The organic phase was dried (sodium sulfate) and the solvent was removed in vacuum. Silicagel chromatography gave 18.9 g of the title compound.

¹H-NMR (400 MHz, DMSO-d6): δ [ppm]=1.34 (d, 3H), 3.55 (s, 3H), 3.79 (q, 1H), 7.08-7.15 (m, 2H), 7.25-7.32 (m, 2H).

### Intermediate Example 08.02

### Rac-2-(4-fluorophenyl)propanoic acid

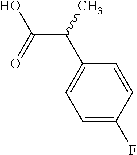

To a stirred solution of rac-methyl 2-(4-fluorophenyl)propanoate (18.9 g) in ethanol (200 mL) was added a solution of potassium hydroxide (35 g), dissolved in water (200 mL). The mixture was stirred at 0° C. for 4 h. Hydrochloric acid (c=4.0 M) was added until pH 5 was reached and the reaction mixture was extracted with ethyl acetate. The organic phase was separated and the solvent was removed in vacuum to give 15.64 g of the title product. The crude product was used without further purification.

¹H-NMR (300 MHz, DMSO-d₆): δ [ppm]=1.31 (d, 3H), 3.66 (q, 1H), 7.05-7.15 (m, 2H), 7.24-7.33 (m, 2H), 12.30 (s, 1H).

### Intermediate Example 08.03

### (2R)-2-(4-fluorophenyl)propanoic acid

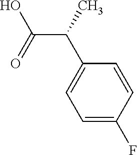

To a stirred solution of Rac-2-(4-fluorophenyl)propanoic acid (23.6 g) in refluxing ethyl acetate (250 mL) was added a solution of (1S)-1-phenylethanamine (17.35 g) in ethyl acetate. The mixture was allowed to cool down to room temperature within 1 h. A white solid was collected by filtration, was washed with ethyl acetate and dried in vacuum to give 27.5 g of a solid. The solid was recrystallized from 400 mL refluxing ethyl acetate. The mixture was allowed to cool down to room temperature. A white solid was collected by filtration, was washed with ethyl acetate and dried in vacuum to give 18.3 g of a solid. The solid was twice recrystallized from refluxing ethyl acetate (350 mL; 300 mL). A white solid was collected by filtration, was washed with ethyl acetate and dried in vacuum to give 10.51 g of a solid. The solid was dissolved in water, hydrochloric acid (c=2.0 M) was added until pH 5 was reached and the reaction mixture was extracted with dichloromethane. The organic phase was dried (sodium sulfate) and the solvent was removed in vacuum to give 5.6 g of the title product. The crude product was used without further purification.

¹H-NMR (300 MHz, DMSO-d₆): δ [ppm]=1.31 (d, 3H), 3.66 (q, 1H), 7.05-7.16 (m, 2H), 7.24-7.33 (m, 2H), 12.28 (br. s., 1H).

[α]D²⁰: −79.3° (in DMSO)

Column: Chiralcel OJ-H 150×4.6; Flow: 1.00 mL/min; Solvent: A: Hexane, B: 2-propanol with 0.1% formic acid; Solvent mixture: 80% A+20% B. Run Time: 30 min. Retention Time: 3.41 min; UV 254 nm; Enantiomeric Ratio: 99.8%: 0.2%.

### Intermediate Example 08.04

### (2R)-2-(4-fluorophenyl)-N-[4-(4,4,5,5-tetramethyl-1,3, 2-dioxaborolan-2-yl)phenyl]propanamide

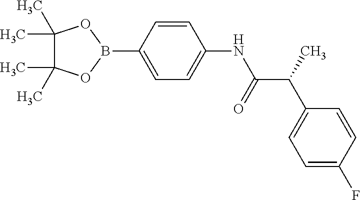 [SMILES: C[C@@H](C(=O)Nc1ccc(B2OC(C)(C)C(C)(C)O2)cc1)c1ccc(F)cc1]

To a stirred solution of 4-(4,4,5,5-tetramethyl-1,3,2-dioxaborolan-2-yl)aniline (1.0 g) in DMF (45 mL) and dichloromethane (90 mL) was added sodium bicarbonate (766 mg), (2R)-2-(4-fluorophenyl)propanoic acid (844 mg) and HATU (2.6 g). The mixture was stirred at room temperature for 4 h. Water was added, and the mixture was stirred for 30 minutes. A half-saturated solution of sodium bicarbonate was added and the mixture was extracted with ethyl acetate. The organic phase was washed with saturated sodium chloride solution, dried (sodium sulfate) and the solvent was removed in vacuum. Silica-gel chromatography gave 1.53 g of the title compound.

¹H-NMR (400 MHz, DMSO-d₆), δ [ppm]=1.23 (12H), 1.37 (3H), 3.74-3.87 (1H), 7.06-7.16 (2H), 7.31-7.42 (2H), 7.51-7.61 (4H), 10.12 (1H).

### Intermediate Example 08.05

### (4-{[(2R)-2-(4-fluorophenyl)propanoyl]amino}phenyl)boronic acid

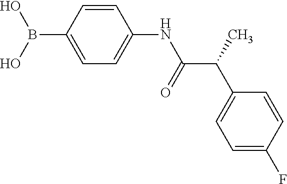 [SMILES: C[C@@H](C(=O)Nc1ccc(B(O)O)cc1)c1ccc(F)cc1]

To a stirred solution of (4-aminophenyl)boronic acid hydrochloride (2.00 g) in DMF (42 mL) was added sodium bicarbonate (2.9 g), (2R)-2-(4-fluorophenyl)propanoic acid (2.04 g) and HATU (6.58 g). The mixture was stirred at room temperature for 72 h. Water (140 mL) was added, and the mixture was stirred for 2 h. The white precipitate was collected by filtration and was washed with water and was dried in vacuum to give 2.86 g of the title compound.

¹H-NMR (300 MHz, DMSO-d₆), δ [ppm]=1.39 (3H), 3.84 (1H), 7.08-7.21 (2H), 7.35-7.44 (2H), 7.52 (2H), 7.69 (2H), 7.88 (2H), 10.07 (1H).

### Intermediate Example 09.01

### (2R)—N-[4-(2-amino[1,2,4]triazolo[1,5-a]pyridin-7-yl)phenyl]-2-(4-fluorophenyl)propanamide

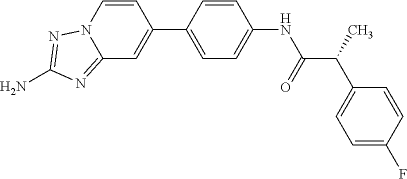 [SMILES: C[C@@H](C(=O)Nc1ccc(-c2ccn3nc(N)nc3c2)cc1)c1ccc(F)cc1]

To a stirred solution of 7-bromo[1,2,4]triazolo[1,5-a]pyridin-2-amine (100 mg; CAS-RN [882521-63-3]; commercially available from Allichem LLC, USA; Baltimore, Md.; preparation described WO2010/020363 A1) in 1-propanol (3 mL) was added potassium carbonate solution (0.7 mL, c=2 M), (4-{[(2R)-2-(4-fluorophenyl)propanoyl]amino}phenyl)boronic acid (202 mg), triphenylphosphine (12 mg) and PdCl₂(PPh₃)₂(33 mg). The mixture was heated to reflux for 16 h. Further triphenylphosphine (12 mg) and PdCl₂(PPh₃)₂(33 mg) were added and the mixture was heated to reflux for further 4 h. The reaction mixture was filtered through an aminophase-silica-gel column and the solvent was removed in vacuum. Silicagel chromatography gave 150 mg of the title compound.

¹H-NMR (400 MHz, DMSO-d₆), δ [ppm]=1.42 (3H), 3.86 (1H), 5.97 (2H), 7.08-7.25 (3H), 7.35-7.49 (2H), 7.58 (1H), 7.63-7.83 (4H), 8.53 (1H), 10.21 (1H).

### Intermediate Example 09.02

### ethyl [(4-chloropyridin-2-yl)carbamothioyl]carbamate

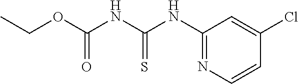 [SMILES: CCOC(=O)NC(=S)Nc1cc(Cl)ccn1]

Ethoxycarbonyl isothiocyanate (11.1 g) was added to a stirred solution of 2-amino-4-chloropyridine (10.1 g) in dioxane (100 mL). The mixture was stirred for 2 h at r.t. A white solid precipitated. Hexane (25 mL) was added and the white solid was collected by filtration to give 8.0 g of the title compound. The solution was concentrated in vacuum and the residue was recrystallized from ethyl acetate to give further 8.5 g of the title compound.

### Intermediate Example 09.03

### 7-chloro[1,2,4]triazolo[1,5-a]pyridin-2-amine

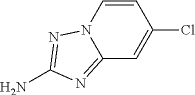 [SMILES: Nc1nc2cc(Cl)ccn2n1]

Hydroxylammonium chloride (13.9 g) was suspended in methanol (70 mL), and ethanol (65 mL) and Hünig Base (21.1 mL) were added at r.t. The mixture was heated to 60° C., ethyl [(4-chloropyridin-2-yl)carbamothioyl]carbamate (9.0 g) was added portionwise, and the mixture was stirred at 60° C. for 2 h. The solvent was removed in vacuum and water (150 mL) was added. A solid was collected by filtration and was washed with ethanol and dried in vacuum. Silicagel chromatography gave 4.2 g of the title compound.

¹H-NMR (300 MHz, DMSO-d₆), δ [ppm]=6.14 (2H), 6.92 (1H), 7.50 (1H), 8.55 (1H).

### Intermediate Example 09.04

### 7-chloro-N-[2-methoxy-4-(methylsulfonyl)phenyl][1,2,4]triazolo[1,5-a]pyridin-2-amine

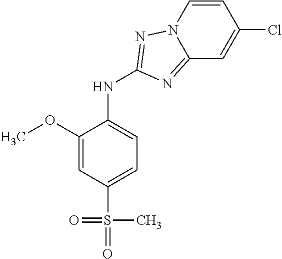 [SMILES: COc1cc(S(C)(=O)=O)ccc1Nc1nc2cc(Cl)ccn2n1]

Starting from 7-chloro[1,2,4]triazolo[1,5-a]pyridin-2-amine (300 mg) and 1-bromo-2-methoxy-4-(methylsulfonyl)benzene (543 mg), Intermediate Example 09.04. was prepared analogously to the procedure for the preparation of Intermediate Example 09.05. Yield: 236 mg of the title compound.

¹H-NMR (300 MHz, DMSO-d₆), δ [ppm]=3.18 (3H), 3.97 (3H), 7.17 (1H), 7.44 (1H), 7.53 (1H), 7.86 (1H), 8.43 (1H), 8.75 (1H), 8.87 (1H).

### Intermediate Example 09.05

### {4-[(7-chloro[1,2,4]triazolo[1,5-a]pyridin-2-yl)amino]-3-methoxyphenyl}(3-fluoroazetidin-1-yl)methanone

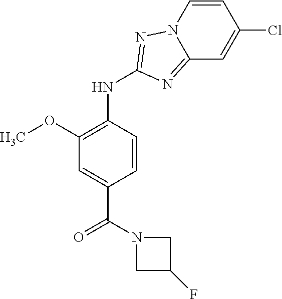 [SMILES: COc1cc(C(=O)N2CC(F)C2)ccc1Nc1nc2cc(Cl)ccn2n1]

To a stirred suspension of 7-chloro[1,2,4]triazolo[1,5-a]pyridin-2-amine (190 mg) in toluene (7 mL) and NMP (0.7 mL) was added (4-Bromo-3-methoxyphenyl)(3-fluoroazetidin-1-yl)methanone (373 mg), chloro(2-dicyclohexylphosphino-2′,4′,6′-tri-iso-propyl-1,1′-biphenyl)[2-(2-aminoethyl) phenyl]palladium(II) methyl-tert-butylether adduct (28 mg), X-Phos (16 mg) and powdered potassium phosphate monohydrate (0.60 g) and the flask was degassed twice and backfilled with argon. The mixture was heated to reflux for 16 h.

A half-saturated solution of potassium carbonate was added and the mixture was extracted with a mixture of dichloromethane and methanol. The organic phase was dried (sodium sulfate) and the solvent was removed in vacuum. The mixture was filtered and concentrated in vacuum. Silicagel chromatography gave 120 mg of the title compound.

¹H-NMR (300 MHz, DMSO-d₆), δ [ppm]=3.91 (3H), 3.94-4.80 (4H), 5.26-5.59 (1H), 7.15 (1H), 7.23-7.33 (2H), 7.82 (1H), 8.21-8.36 (1H), 8.46 (1H), 8.85 (1H).

### Intermediate Example 09.06

### 7-chloro-N-[4-(methylsulfonyl)-2-(2,2,2-trifluoroethoxy)phenyl][1,2,4]triazolo[1,5-a]pyridin-2-amine

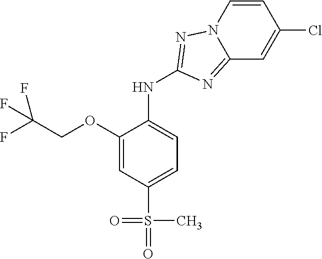 [SMILES: CS(=O)(=O)c1ccc(Nc2nc3cc(Cl)ccn3n2)c(OCC(F)(F)F)c1]

Starting from 7-chloro[1,2,4]triazolo[1,5-a]pyridin-2-amine (100 mg) and 1-bromo-4-(methylsulfonyl)-2-(2,2,2-trifluoroethoxy)benzene (227 mg), Intermediate Example 09.06. was prepared analogously to the procedure for the preparation of Intermediate Example 09.05. Yield: 50 mg of the title compound.

¹H-NMR (400 MHz, DMSO-d₆), δ [ppm]=3.19 (3H), 5.00 (2H), 7.18 (1H), 7.58-7.71 (2H), 7.86 (1H), 8.44 (1H), 8.70 (1H), 8.81-8.92 (1H).

### Intermediate Example 09.07

### {4-[(7-chloro[1,2,4]triazolo[1,5-a]pyridin-2-yl)amino]-3-(2,2,2-trifluoroethoxy)phenyl}(3-fluoroazetidin-1-yl)methanone

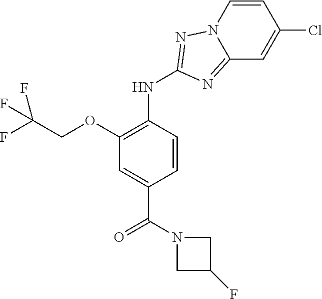 [SMILES: O=C(c1ccc(Nc2nc3cc(Cl)ccn3n2)c(OCC(F)(F)F)c1)N1CC(F)C1]

Starting from 7-chloro[1,2,4]triazolo[1,5-a]pyridin-2-amine (250 mg) and [4-bromo-3-(2,2,2-trifluoroethoxy)phenyl](3-fluoroazetidin-1-yl)methanone (607 mg), Intermediate Example 09.07. was prepared analogously to the procedure for the preparation of Intermediate Example 09.05. Yield: 198 mg of the title compound.

¹H-NMR (400 MHz, DMSO-d₆), δ [ppm]=3.93-4.72 (4H), 4.93 (2H), 5.32-5.55 (1H), 7.16 (1H), 7.36-7.43 (2H), 7.83 (1H), 8.27-8.33 (1H), 8.41 (1H), 8.81-8.90 (1H).

### Intermediate Example 09.08

### azetidin-1-yl{4-[(7-chloro[1,2,4]triazolo[1,5-a]pyridin-2-yl)amino]-3-methoxyphenyl}methanone

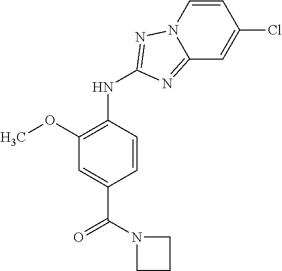 [SMILES: COc1cc(C(=O)N2CCC2)ccc1Nc1nc2cc(Cl)ccn2n1]

Starting from 7-chloro[1,2,4]triazolo[1,5-a]pyridin-2-amine (190 mg) and azetidin-1-yl(4-bromo-3-methoxyphenyl)methanone (350 mg), Intermediate Example 09.08. was prepared analogously to the procedure for the preparation of Intermediate Example 09.05. Yield: 130 mg of the title compound.

¹H-NMR (400 MHz, DMSO-d₆), δ [ppm]=2.27 (2H), 3.88-3.94 (3H), 3.97-4.47 (4H), 7.15 (1H), 7.23-7.31 (2H), 7.83 (1H), 8.28 (1H), 8.42 (1H), 8.79-8.93 (1H).

### Intermediate Example 10.01

### 6-chloro-N-[2-methoxy-4-(methylsulfonyl)phenyl]imidazo[1,2-b]pyridazin-2-amine

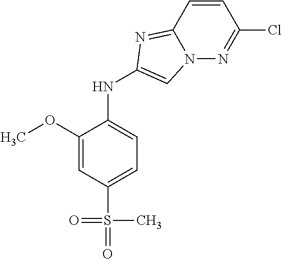 [SMILES: COc1cc(S(C)(=O)=O)ccc1Nc1cn2nc(Cl)ccc2n1]

To a stirred suspension of 6-chloroimidazo[1,2-b]pyridazin-2-amine (250 mg; CAS-RN [887625-09-4]; commercially available from Zylexa Pharma Ltd.; United Kingdom) in toluene (10 mL) and NMP (1.0 mL) was added 1-bromo-2-methoxy-4-(methylsulfonyl)benzene (590 mg), chloro(2-dicyclohexylphosphino-2′,4′,6′-tri-iso-propyl-1,1′-biphenyl)[2-(2-aminoethyl) phenyl]palladium(II) methyl-tert-butylether adduct (123 mg), X-Phos (71 mg) and powdered potassium phosphate monohydrate (1.57 g) and the flask was degassed twice and backfilled with argon. The mixture was heated to reflux for 16 h. The mixture was filtered and concentrated in vacuum. Silicagel chromatography followed by aminophase silicagel chromatography gave 120 mg of the title compound.

¹H-NMR (400 MHz, DMSO-d₆), δ [ppm]=3.15 (3H), 3.99 (3H), 7.26 (1H), 7.40 (1H), 7.46 (1H), 8.01 (1H), 8.05 (1H), 8.53 (1H), 8.92 (1H).

### Intermediate Example 11.01

### 2-chloro-3-methoxy-5-(methylsulfonyl)pyridine

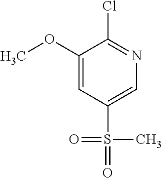

To a solution of sodium sulphite (448 mg) and sodium bicarbonate (313 mg) in water (2.4 ml) was added 6-chloro-5-methoxypyridine-3-sulfonyl chloride (430 mg; CAS-RN [75720-93-3]; commercially available from Ablock Pharmatech, Inc., USA) and ethanol (1.2 mL). The mixture was heated to 50° C. for 45 minutes and concentrated to dryness. The residue was suspended in DMF (3.6 mL), iodomethane (1261 mg) was added and the mixture was stirred at room temperature for 1 hour. The mixture was diluted with water whereby the desired product precipitated. The solid was separated by suction filtration and dried in vacuo to give 265 mg of the title compound.

¹H-NMR (400 MHz, CDCL₃), δ [ppm]=3.16 (3H), 4.04 (3H), 7.66 (1H), 8.55 (1H).

### Intermediate Example 11.02

### 6-bromo-N-[3-methoxy-5-(methylsulfonyl)pyridin-2-yl]imidazo[1,2-a]pyridin-2-amine

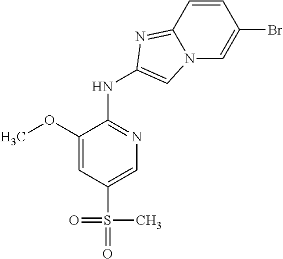 [SMILES: COc1cc(S(C)(=O)=O)cnc1Nc1cn2cc(Br)ccc2n1]

To a stirred suspension of 6-bromoimidazo[1,2-a]pyridin-2-amine hydrochloride (144 mg; CAS-RN [947248-52-4]; commercially available from Apollo Scientific Ltd.; United Kingdom) in THF (10 mL) at 0° C. was added sodium hydride (101 mg; 55%) and the mixture was stirred for 30 minutes. 2-chloro-3-methoxy-5-(methylsulfonyl)pyridine (150 mg) was added and the suspension was heated at 130° C. for 1 hour in a closed microwave vessel in a microwave oven. After cooling, the mixture was diluted with water and extracted with ethyl acetate (3×). The combined organic phases were washed with an aqueous solution of sodium chloride, dried (MgSO₄), filtered and concentrated. Silicagel chromatography gave 75 mg of the title compound.

¹H-NMR (400 MHz, DMSO-d₆), δ [ppm]=3.24 (3H), 3.98 (3H), 7.32 (1H), 7.40 (1H), 7.55 (1H), 8.29 (1H), 8.40 (1H), 8.93 (1H), 9.07 (1H).

### Intermediate Example 12.01

### 5-bromo-6-methoxy-2,3-dihydro-1-benzothiophene

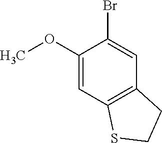

Int12.01 was prepared as described by David W. Robertson et al. in European Journal of Medicinal Chemistry, 1986, 21, p 223-229.

Int12.01 can also be prepared in a similar way as described below:

### Intermediate Example 12.01.a

### 1-[(2,2-dimethoxyethyl)sulfanyl]-3-methoxybenzene

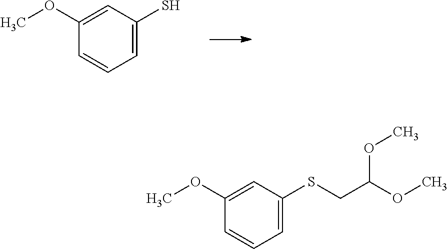

To a stirred solution of 3-methoxybenzenethiol (5.14 g) in acetonitrile (31 mL) was added potassium carbonate (6.08 g) and the mixture was stirred for 2 h at r.t. 2-bromo-1,1-dimethoxyethane (7.67 g) was added and the mixture was stirred for at r.t. for 70 h. Water was added and the mixture was extracted with a mixture of ethyl acetate and hexane (1:1). The organic phase was dried (sodium sulfate) and the solvent was removed in vacuum. Silicagel chromatography gave 8.0 g of the title compound.

¹H-NMR (300 MHz, CHLOROFORM-d), δ [ppm]=3.15 (2H), 3.40 (6H), 3.82 (3H), 4.56 (1H), 6.76 (1H), 6.92-7.01 (2H), 7.19-7.26 (1H).

### Intermediate Example 12.01.b

### 6-methoxy-1-benzothiophene

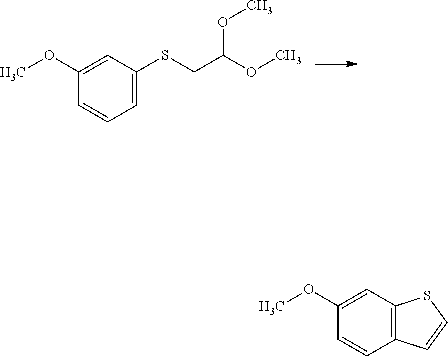

To a stirred solution of 1-[(2,2-dimethoxyethyl)sulfanyl]-3-methoxybenzene (1.0 g) in chlorobenzene (40 mL) was added polyphosphoric acid (1.0 g; CAS-RN: [8017-16-1]; >83% phosphate (as P₂O₅) from Sigma-Aldrich; Order No. 04101) and the mixture was heated to 80° C. for 1 h. The mixture was cooled to 0° C. with an ice-bath and an aqueous solution of sodium hydroxide was added with ice bath cooling until pH7 was reached. The mixture was extracted with dichloromethane, the organic phase was dried (sodium sulfate) and the solvent was removed in vacuum. Silicagel chromatography gave 407 mg of the title compound, containing approx. 20% of a second isomer. This mixture was used for the next step without further purification.

¹H-NMR (400 MHz, DMSO-d₆), δ [ppm]=3.81 (3H), 6.99 (1H), 7.31-7.35 (1H), 7.51 (1H), 7.56 (1H), 7.74 (1H). The product contains approx. 20% of a second isomer.

### Intermediate Example 12.01.c

### 6-methoxy-1-benzothiophene 1,1-dioxide

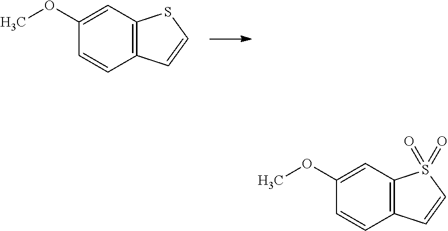

To a stirred solution of 6-methoxy-1-benzothiophene (700 mg) in chloroform (11 mL) at 0° C. was added 3-chlorobenzenecarboperoxoic acid (1.99 g) and the mixture was stirred for 2 h at r.t. An aqueous solution of disodium sulfurothioate was added, the mixture was stirred for 30 minutes and was consecutively extracted with ethyl acetate and with dichloromethane. Both organic phases were washed with a half saturated sodium bicarbonate solution and with saturated sodium chloride solution. The organic phases were combined, dried (sodium sulfate) and the solvent was removed in vacuum. Silicagel chromatography gave 612 mg of the title compound, containing approx. 20% of a second isomer. This mixture was used for the next step without further purification.

¹H-NMR (400 MHz, DMSO-d₆), δ [ppm]=3.86 (3H), 7.15-7.22 (2H), 7.45 (1H), 7.49 (1H), 7.54 (1H).

### Intermediate Example 12.01.d

### 6-methoxy-2,3-dihydro-1-benzothiophene 1,1-dioxide

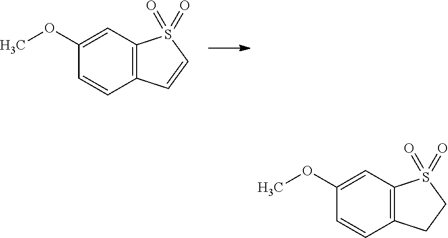

To a stirred solution of 6-methoxy-1-benzothiophene 1,1-dioxide (605 mg) in ethanol (10 mL) and dichloromethane (10 mL) was added palladium on carbon (10% w/w palladium) (147 mg) and the mixture was stirred at r.t. in a hydrogen atmosphere for 16 h. The mixture was filtered, and concentrated in vacuum. Silicagel chromatography gave a solid that was recrystallized from ethanol to give 248 mg of the title compound, as a single isomer.

¹H-NMR (300 MHz, DMSO-d₆), δ [ppm]=3.20-3.29 (2H), 3.53-3.63 (2H), 3.82 (3H), 7.18-7.25 (2H), 7.42 (1H).

### Intermediate Example 12.01.e

### 6-methoxy-2,3-dihydro-1-benzothiophene

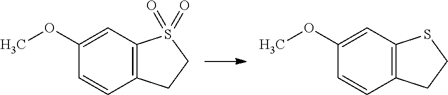

To a stirred solution of 6-methoxy-2,3-dihydro-1-benzothiophene 1,1-dioxide (224 mg) in diethyl ether (80 mL) was added lithium aluminumhydride (386 mg) and the mixture was heated to reflux for 4 h. Water was added, and aqueous hydrochloric acid was added until a clear solution had formed. The mixture was extracted with diethyl ether, the solution was dried (sodium sulfate) and the solvent was removed in vacuum. Silicagel chromatography gave 136 mg of the title compound.

¹H-NMR (300 MHz, DMSO-d₆), δ [ppm]=3.08-3.17 (2H), 3.28-3.37 (2H), 3.69 (3H), 6.55 (1H), 6.81 (1H), 7.11 (1H).

### Intermediate Example 12.01

### 5-bromo-6-methoxy-2,3-dihydro-1-benzothiophene

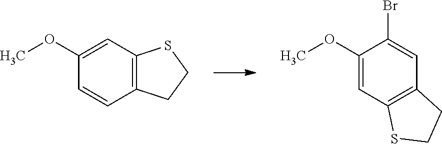

To a stirred solution of 6-methoxy-2,3-dihydro-1-benzothiophene (136 mg) in trichloromethane (9.5 mL) was added a freshly prepared solution of bromine in trichloromethane (0.44 mL; c=10% w/w) at 0° C. and the solution was stirred at 0° C. for 1 h. An aqueous solution of disodium sulfurothioate was added, and the mixture was extracted with dichloromethane. The organic phase was dried (sodium sulfate) and the solvent was removed in vacuum. Silicagel chromatography gave 170 mg of the title compound.

¹H-NMR (400 MHz, DMSO-d₆), δ [ppm]=3.13-3.19 (2H), 3.34-3.40 (2H), 3.78 (3H), 7.03 (1H), 7.33-7.45 (1H).

### Intermediate Example 12.02

### 5-bromo-1,1-dioxido-2, 3-dihydro-1-benzothiophene-6-yl methyl ether

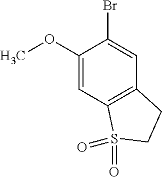

To a stirred solution of 5-bromo-6-methoxy-2,3-dihydro-1-benzothiophene (200 mg) in chloroform (15 mL) was added 3-chlorobenzenecarboperoxoic acid (380 mg) and the mixture was stirred for 1 h at r.t. An aqueous solution of disodium sulfurothioate was added, the mixture was stirred for 30 minutes and was extracted with dichloromethane. The organic phase was washed with a half saturated potassium carbonate solution and with saturated sodium chloride solution, dried (sodium sulfate) and the solvent was removed in vacuum. Silicagel chromatography gave 130 mg of the title compound.

¹H-NMR (400 MHz, DMSO-d₆), δ [ppm]=3.26 (2H), 3.59 (2H), 3.93 (3H), 7.40 (1H), 7.82 (1H).

### Intermediate Example 13.01

### 1-bromo-4-fluoro-2-(2,2,2-trifluoroethoxy)benzene

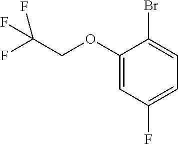 [SMILES: Fc1ccc(Br)c(OCC(F)(F)F)c1]

To a stirred solution of 2-bromo-5-fluorophenol (1.5 g) in acetonitrile (0.5 mL) and DMF (8.5 mL) in a microwave tube was added potassium carbonate (2.1 g) and 2,2,2-trifluoroethyl trifluoromethanesulfonate (2.37 g). The mixture was heated to 150° C. in a microwave oven for 30 minutes. In a second microwave tube the same reaction was repeated. Both mixtures were combined. The solvent was removed in vacuum, ethyl acetate and hexane (1:1) was added and the mixture was washed with water. The organic phase was washed with saturated sodium chloride solution, dried (sodium sulfate) and the solvent was removed in vacuum. Silica gel chromatography gave 4.0 g of the title compound.

¹H-NMR (300 MHz, CHLOROFORM-d): δ [ppm]=4.39 (q, 2H), 6.62-6.78 (m, 2H), 7.53 (dd, 1H).

### Intermediate Example 13.02

### 1-bromo-4-(methylsulfanyl)-2-(2,2,2-trifluoroethoxy)benzene

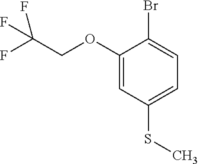

To a stirred solution of 1-bromo-4-fluoro-2-(2,2,2-trifluoroethoxy)benzene (4.0 g) in DMF (15 mL) was added sodium methanethiolate (1.0 g). The mixture was stirred for 2 h at 60° C. The mixture was cooled to room temperature. Water was added and the mixture was extracted with ethyl acetate. The organic phase was washed with saturated sodium chloride solution, dried (sodium sulfate) and the solvent was removed in vacuum to give 3.8 g of the crude title compound, that was used for the next step without purification.

¹H-NMR (300 MHz, CHLOROFORM-d): δ [ppm]=2.48 (s, 3H), 4.39 (q, 2H), 6.78-6.88 (m, 2H), 7.46 (d, 1H).

### Intermediate Example 13.03

### 1-bromo-4-(methylsulfonyl)-2-(2,2,2-trifluoroethoxy)benzene

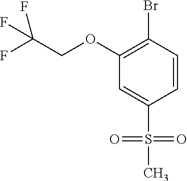 [SMILES: CS(=O)(=O)c1ccc(Br)c(OCC(F)(F)F)c1]

To a stirred solution of 1-bromo-4-(methylsulfanyl)-2-(2,2,2-trifluoroethoxy)benzene (3.8 g) in chloroform (100 mL) was added 3-chlorobenzenecarboperoxoic acid (mCPBA) (8.48 g). The mixture was stirred at room temperature for 16 h. With ice bath cooling, a half-saturated solution of sodium bicarbonate and a 0.2 M solution of sodium thiosulfate was added, the mixture was stirred for 30 minutes and the mixture was extracted with dichloromethane. The organic phase was washed with a 0.2 M solution of sodium thiosulfate and a saturated sodium chloride solution, dried (sodium sulfate) and the solvent was removed in vacuum. Silica gel chromatography gave a solid that was triturated with ether to give 2.1 g of the title compound.

¹H-NMR (400 MHz, CHLOROFORM-d): δ [ppm]=3.06 (s, 3H), 4.50 (q, 2H), 7.45 (d, 1H), 7.52 (dd, 1H), 7.81 (d, 1H).

### Intermediate Example 14.01

### methyl 4-bromo-3-(2,2,2-trifluoroethoxy)benzoate

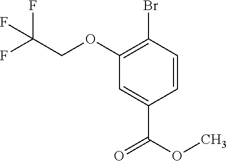 [SMILES: COC(=O)c1ccc(Br)c(OCC(F)(F)F)c1]

To a stirred solution of methyl 4-bromo-3-hydroxybenzoate (2.5 g) in acetonitrile (0.5 mL) and DMF (10 mL) in a microwave tube was added potassium carbonate (2.93 g) and 2,2,2-trifluoroethyl trifluoromethanesulfonate (2.79 g). The mixture was heated to 150° C. in a microwave oven for 30 minutes. The solvent was removed in vacuum, ethyl acetate was added and the mixture was washed with water. The organic phase was washed with saturated sodium chloride solution, dried (sodium sulfate) and the solvent was removed in vacuum. Recrystallization of the residue from ethanol gave 1.2 g of the title compound. The mother liquor was concentrated in vacuum and purified by aminophase-silica-gel chromatography followed by recrystallized from methanol and water to give further 0.64 g of the title compound.

¹H-NMR (300 MHz, CHLOROFORM-d): δ [ppm]=3.93 (s, 3H), 4.47 (q, 2H), 7.56 (d, 1H), 7.58-7.70 (m, 2H).

### Intermediate Example 14.02

### 4-bromo-3-(2,2,2-trifluoroethoxy)benzoic acid

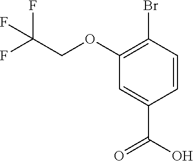 [SMILES: O=C(O)c1ccc(Br)c(OCC(F)(F)F)c1]

To a stirred solution of methyl 4-bromo-3-(2,2,2-trifluoroethoxy)benzoate (1.83 g) in THF (30 mL), methanol (10 mL) and water (10 mL) was added a 1 M solution of lithium hydroxide in water (18 mL). The mixture was stirred at room temperature for 1 h. Water was added and 2 N hydrochloric acid was added until pH 4 was reached. The precipitated solid was collected by filtration, was washed with water. The solid was suspended with toluene and concentrated in vacuum. Trituration of the residue with hexane gave 1.6 g of the title compound.

¹H-NMR (300 MHz, DMSO-d₆): δ [ppm]=4.95 (q, 2H), 7.51 (dd, 1H), 7.65 (d, 1H), 7.74 (d, 1H), 13.29 (br. s., 1H).

### Intermediate Example 14.03

### 4-bromo-3-(2,2,2-trifluoroethoxy)benzamide

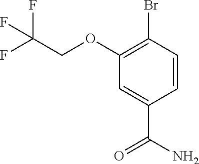 [SMILES: NC(=O)c1ccc(Br)c(OCC(F)(F)F)c1]

To a stirred suspension of 4-bromo-3-(2,2,2-trifluoroethoxy)benzoic acid (0.50 g) in THF (20 mL) was added DMF (0.2 mL) and oxalyl chloride (0.30 mL). The mixture was stirred at room temperature for 0.5 h. With ice bath cooling, ammonia gas was bubbled through the reaction mixture. A white solid precipitated. The mixture was stirred for further 15 minutes. Ethyl acetate was added and the mixture was washed with water and with a saturated solution of sodium chloride. The organic phase was dried (sodium sulfate) and the solvent was removed in vacuum to give a white solid. The solid was triturated with toluene and washed with toluene and hexanes to give 0.27 g of the title compound.

¹H-NMR (300 MHz, DMSO-d₆): δ [ppm]=4.88 (q, 2H), 7.45 (dd, 1H), 7.50 (br. s., 1H), 7.64 (d, 1H), 7.69 (d, 1H), 8.00 (br. s., 1H).

### Intermediate Example 14.04

### [4-bromo-3-(2,2,2-trifluoroethoxy)phenyl](3-fluoroazetidin-1-yl)methanone

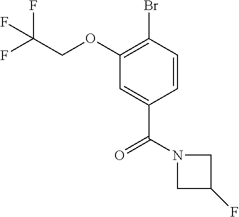 [SMILES: O=C(c1ccc(Br)c(OCC(F)(F)F)c1)N1CC(F)C1]

Starting from 4-bromo-3-(2,2,2-trifluoroethoxy)benzoic acid and 3-fluoroazetidine hydrochloride, Intermediate Example 14.04 was prepared analogously to the procedure for the preparation of Intermediate Example 04.05.

### Examples

### Example 1.1

### 2-(4-fluorophenyl)-N-[4-(2-{[2-methoxy-4-(morpholin-4-ylcarbonyl)phenyl]amino}[1,2,4]triazolo[1,5-b]pyridazin-6-yl)phenyl]acetamide

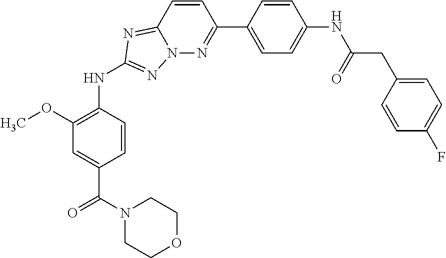 [SMILES: COc1cc(C(=O)N2CCOCC2)ccc1Nc1nc2ccc(-c3ccc(NC(=O)Cc4ccc(F)cc4)cc3)nn2n1]

To a stirred suspension of N-[4-(2-amino[1,2,4]triazolo[1,5-b]pyridazin-6-yl)phenyl]-2-(4-fluorophenyl)acetamide (100 mg) in toluene (2.5 mL) and NMP (1.3 mL) was added (4-bromo-3-methoxyphenyl)(morpholin-4-yl)methanone (124 mg), chloro(2-dicyclohexylphosphino-2′,4′,6′-tri-iso-propyl-1,1′-biphenyl)[2-(2-aminoethyl)phenyl]palladium(II) methyl-tert-butylether adduct (22.8 mg) and X-Phos (13.4 mg) and the flask was degassed twice and backfilled with argon. The mixture was stirred for 5 minutes at room temperature. Powdered potassium phosphate (293 mg) was added and the flask was degassed twice and backfilled with argon. The mixture was heated to reflux for 3 h. The reaction mixture was filtered through an aminophase-silica-gel column and the solvent was removed in vacuum. Aminophase-silica-gel chromatography gave 79 mg of the title compound.

¹H-NMR (400 MHz, DMSO-d₆), δ [ppm]=3.50 (4H), 3.58 (4H), 3.66 (2H), 3.89 (3H), 7.03-7.08 (2H), 7.09-7.18 (2H), 7.31-7.39 (2H), 7.76 (2H), 8.03 (2H), 8.07-8.12 (1H), 8.15-8.21 (1H), 8.26 (1H), 8.46 (1H), 10.38 (1H).

### Example 1.2

### 2-(4-fluorophenyl)-N-[4-(2-{[4-(2-hydroxypropan-2-yl)-2-methoxyphenyl]amino}[1,2,4]triazolo[1, 5-b]pyridazin-6-yl)phenyl]acetamide

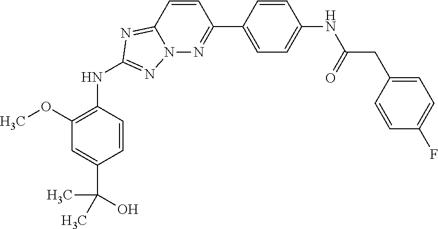 [SMILES: COc1cc(C(C)(C)O)ccc1Nc1nc2ccc(-c3ccc(NC(=O)Cc4ccc(F)cc4)cc3)nn2n1]

To a stirred suspension of N-[4-(2-amino[1,2,4]triazolo[1,5-b]pyridazin-6-yl)phenyl]-2-(4-fluorophenyl)acetamide (100 mg) in toluene (2.5 mL) and NMP (1.3 mL) was added 2-(4-bromo-3-methoxyphenyl)propan-2-ol (101 mg), chloro(2-dicyclohexylphosphino-2′,4′,6′-tri-iso-propyl-1,1′-biphenyl) [2-(2-aminoethyl)phenyl]palladium(II) methyl-tert-butylether adduct (22.8 mg) and X-Phos (13.4 mg) and the flask was degassed twice and backfilled with argon. The mixture was stirred for 5 minutes at room temperature. Powdered potassium phosphate (293 mg) was added and the flask was degassed twice and backfilled with argon. The mixture was heated to reflux for 3 h. The reaction mixture was filtered through an aminophase-silica-gel column and the solvent was removed in vacuum. Aminophase-silica-get chromatography gave 90 mg of the title compound.

¹H-NMR (300 MHz, DMSO-d₆), δ [ppm]=1.41 (6H), 3.66 (2H), 3.84 (3H), 4.93 (1H), 7.01 (1H), 7.07-7.18 (3H), 7.29-7.40 (2H), 7.75 (2H), 7.96-8.09 (4H), 8.09-8.17 (2H), 10.39 (1H).

### Example 1.3

### 2-(4-fluorophenyl)-N-[4-(2-{[2-methoxy-4-(methylsulfonyl)phenyl]amino}[1,2,4]triazolo[1, 5-b]pyridazin-6-yl)phenyl]acetamide

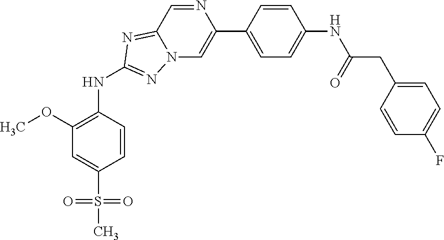 [SMILES: COc1cc(S(C)(=O)=O)ccc1Nc1nc2cnc(-c3ccc(NC(=O)Cc4ccc(F)cc4)cc3)cn2n1]

To a stirred suspension of N-[4-(2-amino[1,2,4]triazolo[1,5-b]pyridazin-6-yl)phenyl]-2-(4-fluorophenyl)acetamide (100 mg) in toluene (2.5 mL) and NMP (1.3 mL) was added 1-bromo-2-methoxy-4-(methylsulfonyl)benzene (110 mg), chloro(2-dicyclohexylphosphino-2′,4′,6′-tri-iso-propyl-1,1′-biphenyl)[2-(2-aminoethyl)phenyl]palladium(II) methyl-tert-butylether adduct (22.8 mg) and X-Phos (13.4 mg) and the flask was degassed twice and backfilled with argon. The mixture was stirred for 5 minutes at room temperature. Powdered potassium phosphate (293 mg) was added and the flask was degassed twice and backfitted with argon. The mixture was heated to reflux for 3 h. The reaction mixture was filtered through an aminophase-silica-gel column and the solvent was removed in vacuum. Aminophase-silica-gel chromatography gave 90 mg of the title compound.

¹H-NMR (400 MHz, DMSO-d₆), δ [ppm]=3.17 (3H), 3.66 (2H), 3.96 (3H), 7.08-7.17 (2H), 7.31-7.39 (2H), 7.44 (1H), 7.55 (1H), 7.76 (2H), 8.04 (2H), 8.13 (1H), 8.19-8.27 (1H), 8.46 (1H), 8.85 (1H), 10.40 (1H).

### Example 2.1

### N-(4-{2-[(2-cyanophenyl)amino][1,2,4]triazolo[1,5-a]pyrazin-6-yl}phenyl)-2-(4-fluorophenyl)acetamide

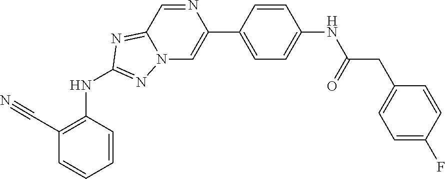 [SMILES: N#Cc1ccccc1Nc1nc2cnc(-c3ccc(NC(=O)Cc4ccc(F)cc4)cc3)cn2n1]

To a stirred suspension of N-[4-(2-amino[1,2,4]triazolo[1,5-a]pyrazin-6-yl)phenyl]-2-(4-fluorophenyl)acetamide (4.00 g) in toluene (100 mL) and NMP (8.0 mL) was added 2-bromobenzonitrile (4.10 g), (R)-BINAP (1.37 g) and Pd₂dba₃ (1.01 g) and cesium carbonate (17.98 g) and the flask was degassed twice and backfilled with argon. The mixture was heated to reflux for 3 h. Water was added and the reaction mixture was extracted with ethyl acetate. The organic phase was washed with saturated sodium chloride solution, dried (sodium sulfate) and the solvent was removed in vacuum. Silicagel chromatography gave a solid that was triturated with dichloromethane and afterwards with hot ethyl acetate to give 1.88 g of the crude title compound as a solid. The solid was dissolved in DMF (50 mL). Ethyl acetate (300 mL) was added and the organic phase was washed with a half-saturated sodium chloride solution for three times. The compound precipitated in the organic phase and was collected by filtration. The solid was washed with dichloromethane and hexane and dried in vacuum to give 1.65 g of the title compound.

¹H-NMR (400 MHz, DMSO-d₆), δ [ppm]=3.65 (2H), 7.09-7.16 (2H), 7.20 (1H), 7.31-7.38 (2H), 7.62-7.72 (3H), 7.76 (1H), 7.94 (1H), 8.01-8.08 (2H), 9.10 (1H), 9.43 (1H), 9.90 (1H), 10.33 (1H).

### Example 2.2

### N-(4-{2-[(2-cyano-3-fluorophenyl)amino][1,2,4]triazolo[1,5-a]pyrazin-6-yl}phenyl)-2-(4-fluorophenyl)acetamide

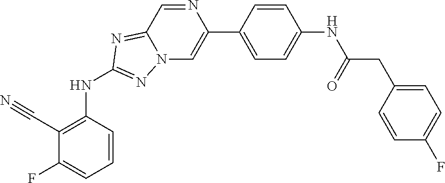 [SMILES: N#Cc1c(F)cccc1Nc1nc2cnc(-c3ccc(NC(=O)Cc4ccc(F)cc4)cc3)cn2n1]

To a stirred suspension of N-[4-(2-amino[1,2,4]triazolo[1,5-a]pyrazin-6-yl)phenyl]-2-(4-fluorophenyl)acetamide (200 mg) in toluene (2 mL) and NMP (0.2 mL) was added 2-bromo-6-fluorobenzonitrile (227 mg), (rac)-BINAP (35 mg) and Pd₂dba₃ (25 mg) and cesium carbonate (551 mg) and the flask was degassed twice and backfilled with argon. The mixture was heated to reflux for 4 h. A mixture of ethyl acetate and methanol (100:1; 250 mL) was added and the mixture was filtered through celite. The organic phase was washed with saturated sodium bicarbonate solution, with saturated sodium chloride solution, dried (sodium sulfate) and the solvent was removed in vacuum. Repeated silicagel chromatography gave a solid that was triturated with warm ethanol to give 31 mg of the title compound.

¹H-NMR (300 MHz, DMSO-d₆), δ [ppm]=3.64 (2H), 7.08-7.18 (3H), 7.30-7.39 (2H), 7.65-7.75 (3H), 7.80-7.87 (1H), 8.05 (2H), 9.15 (1H), 9.46 (1H), 10.23 (1H), 10.31 (1H).

### Example 2.3

### N-(4-{2-[(2-cyanophenyl)amino][1,2,4]triazolo[1,5-a]pyrazin-6-yl}phenyl)-2-phenylacetamide

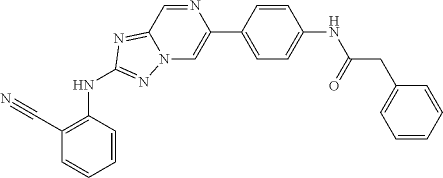 [SMILES: N#Cc1ccccc1Nc1nc2cnc(-c3ccc(NC(=O)Cc4ccccc4)cc3)cn2n1]

To a stirred solution of 2-{[6-(4-aminophenyl)[1,2,4]triazolo[1,5-a]pyrazin-2-yl]amino}benzonitrile (70 mg) in DMF (2.1 mL) was added potassium carbonate (118 mg), phenylacetic acid (43.7 mg) and TBTU (206 mg). The mixture was stirred at room temperature for 64 h. Water was added, the mixture was stirred for 15 minutes and the mixture was extracted with dichloromethane and methanol (100:1). The organic phase was washed with saturated sodium bicarbonate solution dried (sodium sulfate) and the solvent was removed in vacuum. Repeated silicagel chromatography followed by preparative reverse phase HPLC gave a solid that was triturated with warm ethanol to give 11 mg of the title compound.

¹H-NMR (300 MHz, DMSO-d₆), δ [ppm]=3.64 (2H), 7.16-7.25 (2H), 7.26-7.35 (4H), 7.62-7.73 (3H), 7.76 (1H), 7.94 (1H), 8.04 (2H), 9.11 (1H), 9.43 (1H), 9.90 (1H), 10.31 (1H).

### Example 2.4

### 2-(4-fluorophenyl)-N-[4-(2-{[2-methoxy-4-(4-methylpiperazin-1-yl)phenyl]amino}[1,2,4]triazolo[1,5-a]pyrazin-6-yl)phenyl]acetamide

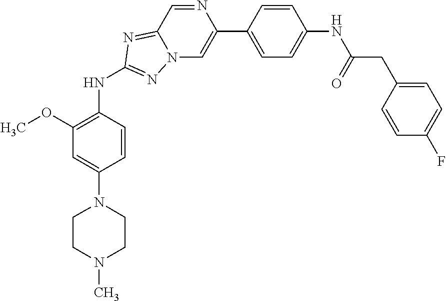 [SMILES: COc1cc(N2CCN(C)CC2)ccc1Nc1nc2cnc(-c3ccc(NC(=O)Cc4ccc(F)cc4)cc3)cn2n1]

To a stirred suspension of N-[4-(2-amino[1,2,4]triazolo[1,5-a]pyrazin-6-yl)phenyl]-2-(4-fluorophenyl)acetamide (150 mg) in toluene (7.0 mL) and NMP (3.4 mL) was added 1-(4-Bromo-3-methoxyphenyl)-4-methylpiperazine (236 mg), chloro(2-dicyclohexylphosphino-2′,4′,6′-tri-iso-propyl-1,1′-biphenyl)[2-(2-aminoethyl)phenyl]palladium(II) methyl-tert-butylether adduct (34.2 mg) and X-Phos (20.1 mg) and the flask was degassed twice and backfilled with argon. The mixture was stirred for 5 minutes at room temperature. Sodium 2-methylpropan-2-olate (199 mg) was added and the flask was degassed twice and backfilled with argon. The mixture was heated to reflux for 2 h. Water was added and the reaction mixture was extracted with ethyl acetate and methanol (10:1). The organic phase was washed with saturated sodium chloride solution, dried (sodium sulfate) and the solvent was removed in vacuum. Repeated aminophase-silica-gel chromatography gave a solid that was triturated with dichloromethane to give 28 mg of the title compound.

¹H-NMR (400 MHz, DMSO-d₆), δ [ppm]=2.19 (3H), 2.40-2.45 (4H), 3.02-3.12 (4H), 3.64 (2H), 3.79 (3H), 6.48 (1H), 6.62 (1H), 7.08-7.17 (2H), 7.30-7.38 (2H), 7.68 (2H), 7.72-7.77 (1H), 8.02 (2H), 8.20 (1H), 8.93-9.02 (1H), 9.35 (1H), 10.30 (1H).

### Example 2.5

### N-(4-{2-[(2-cyano-3-fluorophenyl)amino][1,2,4]triazolo[1,5-a]pyrazin-6-yl}phenyl)-2-phenylacetamide

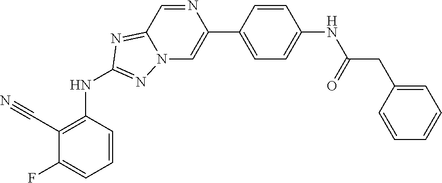 [SMILES: N#Cc1c(F)cccc1Nc1nc2cnc(-c3ccc(NC(=O)Cc4ccccc4)cc3)cn2n1]

To a stirred suspension of N-[4-(2-amino[1,2,4]triazolo[1,5-a]pyrazin-6-yl)phenyl]-2-phenylacetamide (330 mg) in toluene (3.5 mL) and NMP (0.35 mL) was added 2-bromo-6-fluorobenzonitrile (395 mg), (rac)-BINAP (61 mg) and Pd₂dba₃ (44 mg) and cesium carbonate (956 mg) and the flask was degassed twice and backfilled with argon. The mixture was heated to reflux for 4 h. A mixture of ethyl acetate and methanol (100:1; 250 mL) was added and the mixture was filtered through celite. The organic phase was washed with saturated sodium bicarbonate solution, with saturated sodium chloride solution, dried (sodium sulfate) and the solvent was removed in vacuum. Silicagel chromatography followed by repeated aminophase-silica-gel chromatography gave a solid that was dissolved in DMF/THF/methanol and was precipitated by adding this solution to excess water. The precipitate was collected by filtration, was washed with water, ethanol and ether and was dried in vacuum to give 52 mg of the title compound.

¹H-NMR (400 MHz, DMSO-d₆), δ [ppm]=3.64 (2H), 7.13 (1H), 7.19-7.25 (1H), 7.26-7.35 (4H), 7.65-7.75 (3H), 7.84 (1H), 8.05 (2H), 9.15 (1H), 9.47 (1H), 10.24 (1H), 10.32 (1H).

### Example 2.6

### N-(4-{2-[(2-cyanophenyl)amino][1,2,4]triazolo[1,5-a]pyrazin-6-yl}phenyl)-2-(3,4-difluorophenyl)acetamide

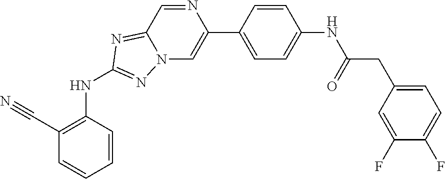 [SMILES: N#Cc1ccccc1Nc1nc2cnc(-c3ccc(NC(=O)Cc4ccc(F)c(F)c4)cc3)cn2n1]

To a stirred suspension of N-[4-(2-amino[1,2,4]triazolo[1,5-a]pyrazin-6-yl)phenyl]-2-(3,4-difluorophenyl)acetamide (270 mg) in toluene (4.0 mL) and NMP (0.4 mL) was added 2-bromobenzonitrile (196 mg), (rac)-BINAP (45.1 mg) and Pd₂dba₃ (32.5 mg) and cesium carbonate (708 mg) and the flask was degassed twice and backfilled with argon. The mixture was heated to reflux for 4 h. Ethyl acetate and methanol (100:1) was added and the mixture was filtered through a silica-gel column and the solvent was removed in vacuum. Aminophase-silica-gel chromatography gave a solid that was triturated with ethanol to give a solid. The solid was dissolved in DMF and THF (1:1) and was precipitated by adding this solution to excess water. The precipitate was collected by filtration, was washed with water, ethanol and ether and was dried in vacuum to give a solid that was recrystallized from ethanol to give 17 mg of the title compound.

¹H-NMR (400 MHz, DMSO-d₆), δ [ppm]=3.68 (2H), 7.11-7.25 (2H), 7.31-7.42 (2H), 7.61-7.72 (3H), 7.76 (1H), 7.94 (1H), 8.05 (2H), 9.11 (1H), 9.43 (1H), 9.89 (1H), 10.31 (1H).

### Example 2.7

### N-(4-{2-[(2-cyano-3-fluorophenyl)amino][1,2,4]triazolo[1,5-a]pyrazin-6-yl}phenyl)-2-(3,4-difluorophenyl)acetamide

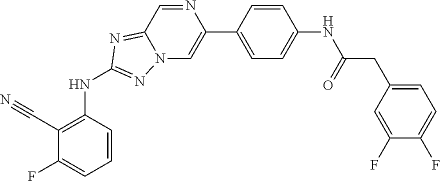 [SMILES: N#Cc1c(F)cccc1Nc1nc2cnc(-c3ccc(NC(=O)Cc4ccc(F)c(F)c4)cc3)cn2n1]

To a stirred suspension of N-[4-(2-amino[1,2,4]triazolo[1,5-a]pyrazin-6-yl)phenyl]-2-(3,4-difluorophenyl)acetamide (270 mg) in toluene (4.0 mL) and NMP (0.4 mL) was added 2-bromo-6-fluorobenzonitrile (220 mg), (rac)-BINAP (45.1 mg) and Pd₂dba₃ (32.5 mg) and cesium carbonate (708 mg) and the flask was degassed twice and backfilled with argon. The mixture was heated to reflux for 4 h. Ethyl acetate and methanol (100:1) was added and the mixture was filtered through a silica-gel column and the solvent was removed in vacuum. Aminophase-silica-gel chromatography gave a solid that was triturated with ethanol to give a solid. The solid was dissolved in DMF and THF (1:1) and was precipitated by adding this solution to excess water. The precipitate was collected by filtration, was washed with water, ethanol and ether and was dried in vacuum to give 80 mg of the title compound.

¹H-NMR (300 MHz, DMSO-d₆), δ [ppm]=3.67 (2H), 7.06-7.19 (2H), 7.29-7.43 (2H), 7.63-7.76 (3H), 7.78-7.88 (1H), 8.06 (2H), 9.15 (1H), 9.46 (1H), 10.22 (1H), 10.32 (1H).

### Example 2.8

### 2-(4-fluorophenyl)-N-[4-(2-{[2-methoxy-4-(methylsulfonyl)phenyl]amino}[1,2,4]triazolo[1,5-a]pyrazin-6-yl)phenyl]acetamide

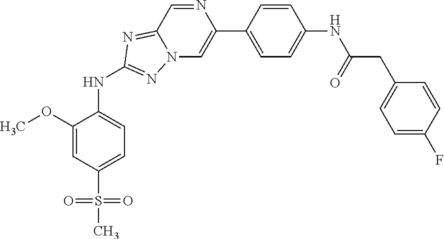 [SMILES: COc1cc(S(C)(=O)=O)ccc1Nc1nc2cnc(-c3ccc(NC(=O)Cc4ccc(F)cc4)cc3)cn2n1]

To a stirred suspension of N-[4-(2-amino[1,2,4]triazolo[1,5-a]pyrazin-6-yl)phenyl]-2-(4-fluorophenyl)acetamide (100 mg) in toluene (3.0 mL) and NMP (1.5 mL) was added 1-bromo-2-methoxy-4-(methylsulfonyl)benzene (146 mg), chloro(2-dicyclohexylphosphino-2′,4′,6′-tri-iso-propyl-1,1′-biphenyl)[2-(2-aminoethyl)phenyl]palladium(II) methyl-tert-butylether adduct (22.8 mg) and X-Phos (13.4 mg) and the flask was degassed twice and backfilled with argon. The mixture was stirred for 5 minutes at room temperature. Powdered potassium phosphate (293 mg) was added and the flask was degassed twice and backfitted with argon. The mixture was heated to reflux for 2 h. The reaction mixture was filtered through an aminophase-silica-gel column and the solvent was removed in vacuum. Aminophase-silica-gel chromatography gave 88 mg of the title compound.

¹H-NMR (400 MHz, DMSO-d₆), δ [ppm]=3.17 (3H), 3.65 (2H), 3.95 (3H), 7.06-7.19 (2H), 7.35 (2H), 7.44 (1H), 7.53 (1H), 7.70 (2H), 8.07 (2H), 8.46 (1H), 9.06 (1H), 9.17 (1H), 9.47 (1H), 10.32 (1H).

### Example 3.1

### 2-{[6-(4-hydroxy-3,5-dimethylphenyl)[1,2,4]triazolo[1,5-a]pyrazin-2-yl]amino}benzonitrile

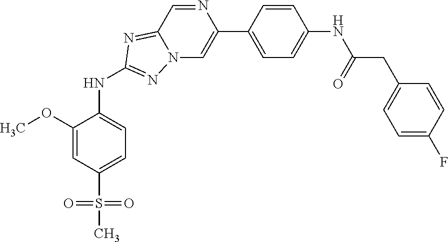 [SMILES: COc1cc(S(C)(=O)=O)ccc1Nc1nc2cnc(-c3ccc(NC(=O)Cc4ccc(F)cc4)cc3)cn2n1]

To a stirred suspension of 4-(2-amino[1,2,4]triazolo[1,5-a]pyrazin-6-yl)-2,6-dimethylphenol (90 mg) in toluene (3.0 mL) and NMP (0.3 mL) was added 2-bromobenzonitrile (97.2 mg), (rac)-BINAP (22.4 mg) and Pd₂dba₃ (16.1 mg) and cesium carbonate (352 mg) and the flask was degassed twice and backfilled with argon. The mixture was heated to reflux for 5 h. Ethyl acetate and methanol (100:1) was added and the mixture was filtered through celite. The organic phase was washed with saturated sodium bicarbonate solution, with saturated sodium chloride solution, dried (sodium sulfate) and the solvent was removed in vacuum. Silicagel chromatography gave a solid that was triturated with warm ethanol to give 80 mg of the title compound.

¹H-NMR (300 MHz, DMSO-d₆), δ [ppm]=2.14-2.25 (6H), 7.19 (1H), 7.61-7.71 (3H), 7.75 (1H), 7.94 (1H), 8.52 (1H), 9.06 (1H), 9.30 (1H), 9.86 (1H).

### Example 3.2

### N-(4-{2-[(2-cyanophenyl)amino][1,2,4]triazolo[1,5-a]pyrazin-6-yl}phenyl)-2-cyclopropylacetamide

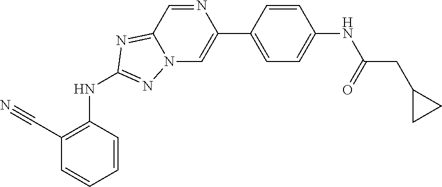 [SMILES: N#Cc1ccccc1Nc1nc2cnc(-c3ccc(NC(=O)CC4CC4)cc3)cn2n1]

To a stirred suspension of N-[4-(2-amino[1,2,4]triazolo[1,5-a]pyrazin-6-yl)phenyl]-2-cyclopropylacetamide (140 mg) in toluene (1.65 mL) and NMP (0.165 mL) was added 2-bromobenzonitrile (125 mg), (rac)-BINAP (28.8 mg) and Pd₂dba₃ (20.8 mg) and cesium carbonate (453 mg) and the flask was degassed twice and backfilled with argon. The mixture was heated to reflux for 4 h. Ethyl acetate and methanol (100:1) was added and the mixture was filtered through a silica-gel column and the solvent was removed in vacuum. Aminophase-silica-gel chromatography gave a solid that was triturated with ethanol to give a solid. The solid was dissolved in DMF and THF (1:1), filtered and was precipitated by adding this solution to excess water. The precipitate was collected by filtration, was washed with water, ethanol and ether and was dried in vacuum to give 87 mg of the title compound.

¹H-NMR (400 MHz, DMSO-d₆), δ [ppm]=0.13-0.22 (2H), 0.41-0.51 (2H), 0.98-1.11 (1H), 2.21 (2H), 7.21 (1H), 7.63-7.72 (3H), 7.76 (1H), 7.94 (1H), 7.99-8.08 (2H), 9.11 (1H), 9.44 (1H), 9.91 (1H), 9.95 (1H).

### Example 3.3

### 3-{2-[(2-cyanophenyl)amino][1,2,4]triazolo[1,5-a]pyrazin-6-yl}-N-cyclopropylbenzamide

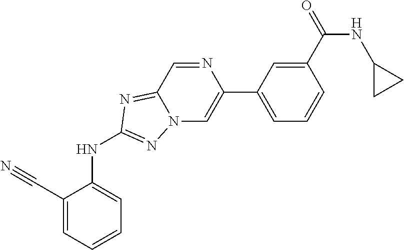 [SMILES: N#Cc1ccccc1Nc1nc2cnc(-c3cccc(C(=O)NC4CC4)c3)cn2n1]

To a stirred suspension of 3-{2-[(2-cyanophenyl)amino][1,2,4]triazolo[1,5-a]pyrazin-6-yl}benzoic acid (88 mg) in THF (3.0 mL) was added Hünig Base (46 μL), cyclopropanamine (19 μL), and HATU (103 mg). The mixture was stirred at room temperature for 16 h. Water was added and the mixture was stirred at room temperature for 15 minutes. The solvent was removed in vacuum and the residue was triturated with methanol to give 56 mg of the title compound.

¹H-NMR (300 MHz, DMSO-d₆), δ [ppm]=0.53-0.61 (2H), 0.65-0.74 (2H), 2.85 (1H), 7.22 (1H), 7.55 (1H), 7.67 (1H), 7.77 (1H), 7.83 (1H), 7.94 (1H), 8.22 (1H), 8.45-8.59 (2H), 9.17 (1H), 9.56 (1H), 9.96 (1H).

### Example 3.4

### 3-{2-[(2-cyanophenyl)amino][1,2,4]triazolo[1,5-a]pyrazin-6-yl}-N-ethylbenzamide

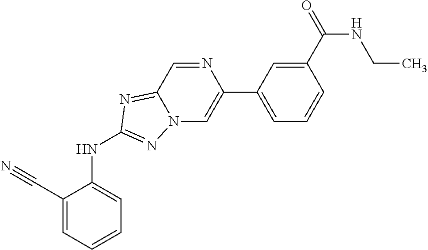 [SMILES: CCNC(=O)c1cccc(-c2cn3nc(Nc4ccccc4C#N)nc3cn2)c1]

To a stirred suspension of 3-{2-[(2-cyanophenyl)amino][1,2,4]triazolo[1,5-a]pyrazin-6-yl}benzoic acid (133 mg) in THF (5.0 mL) was added Hünig Base (70 μL), ethanamine (205 μL; solution in THF, c=2M), and HATU (156 mg). The mixture was stirred at room temperature for 64 h. Water was added and the mixture was stirred at room temperature for 1 h. The precipitated solid was collected by filtration, was washed with ethanol and ether and was dried in vacuum to give 130 mg of the title compound.

¹H-NMR (300 MHz, DMSO-d₆, detected signals), δ [ppm]=1.13 (3H), 7.22 (1H), 7.56 (1H), 7.67 (1H), 7.77 (1H), 7.85 (1H), 7.94 (1H), 8.22 (1H), 8.49-8.61 (2H), 9.17 (1H), 9.57 (1H), 9.97 (1H).

### Example 3.5

### 3-{2-[(2-cyanophenyl)amino][1,2,4]triazolo[1,5-a]pyrazin-6-yl}-N-cyclopentylbenzamide

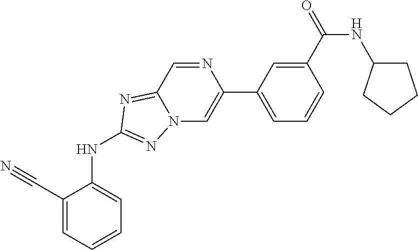 [SMILES: N#Cc1ccccc1Nc1nc2cnc(-c3cccc(C(=O)NC4CCCC4)c3)cn2n1]

To a stirred suspension of 3-{2-[(2-cyanophenyl)amino][1,2,4]triazolo[1,5-a]pyrazin-6-yl}benzoic acid (133 mg) in THF (5.0 mL) was added Hünig Base (70 μL), cyclopentanamine (40 μL), and HATU (156 mg). The mixture was stirred at room temperature for 64 h. Water was added and the mixture was stirred at room temperature for 1 h. The precipitated solid was collected by filtration, was washed with ethanol and ether and was dried in vacuum to give 140 mg of the title compound.

¹H-NMR (300 MHz, DMSO-d₆), δ [ppm]=1.40-1.61 (4H), 1.62-1.77 (2H), 1.81-1.99 (2H), 4.13-4.33 (1H), 7.22 (1H), 7.55 (1H), 7.67 (1H), 7.77 (1H), 7.85 (1H), 7.94 (1H), 8.17-8.26 (1H), 8.37 (1H), 8.50 (1H), 9.18 (1H), 9.59 (1H), 9.96 (1H).

### Example 3.6

### N-(4-{2-[(2-cyano-3-fluorophenyl)amino][1,2,4]triazolo[1,5-a]pyrazin-6-yl}phenyl)-2-cyclopropylacetamide

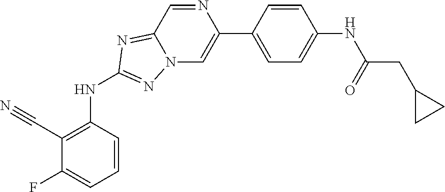 [SMILES: N#Cc1c(F)cccc1Nc1nc2cnc(-c3ccc(NC(=O)CC4CC4)cc3)cn2n1]

To a stirred suspension of N-[4-(2-amino[1,2,4]triazolo[1,5-a]pyrazin-6-yl)phenyl]-2-cyclopropylacetamide (280 mg) in toluene (3.3 mL) and NMP (0.33 mL) was added 2-bromo-6-fluorobenzonitrile (280 mg), (rac)-BINAP (57.7 mg) and Pd₂dba₃ (41.6 mg) and cesium carbonate (906 mg) and the flask was degassed twice and backfilled with argon. The mixture was heated to reflux for 4 h. Ethyl acetate and methanol (100:1) was added and the mixture was filtered through celite and through a silica-gel column and the solvent was removed in vacuum. The residue was triturated with ethanol to give a solid. The solid was dissolved in DMF and THF (1:1) and was precipitated by adding this solution to excess water. The precipitate was collected by filtration, was washed with water, ethanol and ether and was dried in vacuum to give a solid that was recrystallized from ethanol to give 258 mg of the title compound.

¹H-NMR (300 MHz, DMSO-d₆), δ [ppm]=0.10-0.24 (2H), 0.38-0.52 (2H), 0.94-1.13 (1H), 2.20 (2H), 7.07-7.19 (1H), 7.63-7.77 (3H), 7.80-7.88 (1H), 8.05 (2H), 9.15 (1H), 9.46 (1H), 9.95 (1H), 10.23 (1H).

### Example 3.7

### 2-{[6-(4-aminophenyl)[1,2,4]triazolo[1,5-a]pyrazin-2-yl]amino}benzonitrile

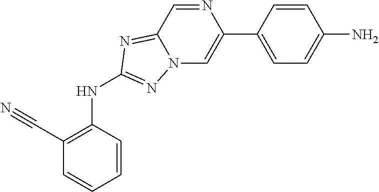 [SMILES: N#Cc1ccccc1Nc1nc2cnc(-c3ccc(N)cc3)cn2n1]

To a stirred suspension of tert-butyl(4-{2-[(2-cyanophenyl)amino][1,2,4]triazolo[1,5-a]pyrazin-6-yl}phenyl)carbamate (1.3 g) in dichloromethane (65 mL) was added 1,3 dimethoxybenzene (3.89 mL) and glacial acetic acid (43 mL). The mixture was stirred at room temperature until a clear solution had formed. The solution was cooled to 0° C. and borontrifluoride diethyletherat (1.54 mL) was added. The mixture was stirred at r.t. for 2 h. An aqueous solution of potassium carbonate was added until pH 11 was reached and the mixture was extracted with ethyl acetate. The organic phase was washed with saturated sodium chloride solution, dried (sodium sulfate) and the solvent was removed in vacuum. Silicagel chromatography gave 120 mg of the title compound.

¹H-NMR (400 MHz, DMSO-d₆), δ [ppm]=5.40 (2H), 6.61 (2H), 7.19 (1H), 7.66 (1H), 7.72-7.82 (3H), 7.94 (1H), 9.03 (1H), 9.21 (1H), 9.81 (1H).

### Example 3.8

### 4-{2-[(2-methoxyphenyl)amino][1,2,4]triazolo[1,5-a]pyrazin-6-yl}-2,6-dimethylphenol

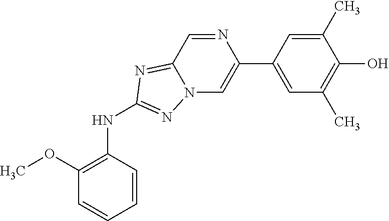 [SMILES: COc1ccccc1Nc1nc2cnc(-c3cc(C)c(O)c(C)c3)cn2n1]

To a stirred suspension of 4-(2-amino[1,2,4]triazolo[1,5-a]pyrazin-6-yl)-2,6-dimethylphenol (160 mg) in toluene (5.3 mL) and NMP (0.53 mL) was added 1-bromo-2-methoxybenzene (0.16 mL), (rac)-BINAP (39.8 mg) and Pd₂dba₃ (28.7 mg) and cesium carbonate (612 mg) and the flask was degassed twice and backfilled with argon. The mixture was heated to reflux for 5 h. Ethyl acetate and methanol (100:1) was added and the mixture was filtered through celite. The organic phase was washed with saturated sodium bicarbonate solution, with saturated sodium chloride solution, was dried (sodium sulfate) and the solvent was removed in vacuum. Silicagel chromatography gave a solid that was triturated with a mixture of diisopropyl ether and ethanol to give 9 mg of the title compound.

¹H-NMR (300 MHz, DMSO-d₆), δ [ppm]=2.21 (6H), 3.84 (3H), 6.88-7.08 (3H), 7.69 (2H), 8.08-8.22 (1H), 8.36 (1H), 8.51 (1H), 9.03 (1H), 9.29 (1H).

### Example 3.9

### 3-{2-[(2-cyanophenyl)amino][1,2,4]triazolo[1,5-a]pyrazin-6-yl}-N-cyclohexylbenzamide

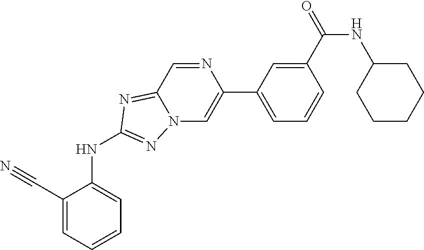 [SMILES: N#Cc1ccccc1Nc1nc2cnc(-c3cccc(C(=O)NC4CCCCC4)c3)cn2n1]

To a stirred suspension of 3-{2-[(2-cyanophenyl)amino][1,2,4]triazolo[1,5-a]pyrazin-6-yl}benzoic acid (133 mg) in THF (5.0 mL) was added Hünig Base (70 μL), cyclohexanamine (41 μL), and HATU (156 mg). The mixture was stirred at room temperature for 64 h. Water was added and the mixture was stirred at room temperature for 1 h. The precipitated solid was collected by filtration, was washed with ethanol and ether and was dried in vacuum to give 140 mg of a solid that was triturated with dichloromethane to give 109 mg the title compound.

¹H-NMR (400 MHz, METHANOL-d₄), δ [ppm]=1.85-1.99 (1H), 2.02-2.19 (4H), 2.41 (1H), 2.53 (2H), 2.65 (2H), 4.49-4.67 (1H), 8.03 (1H), 8.37 (1H), 8.48 (1H), 8.58 (1H), 8.64-8.69 (1H), 8.75 (1H), 9.00-9.06 (1H), 9.10 (1H), 9.26-9.36 (1H), 9.99 (1H), 10.40 (1H), 10.78 (1H).

### Example 4.1

### 2-(4-fluorophenyl)-N-[4-(2-{[2-methoxy-4-(morpholin-4-ylcarbonyl)phenyl]amino}-1,3-benzothiazol-6-yl)phenyl]acetamide

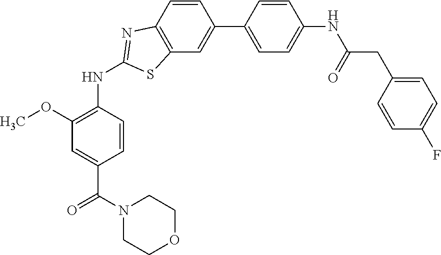 [SMILES: COc1cc(C(=O)N2CCOCC2)ccc1Nc1nc2ccc(-c3ccc(NC(=O)Cc4ccc(F)cc4)cc3)cc2s1]

To a stirred suspension of N-[4-(2-amino-1,3-benzothiazol-6-yl)phenyl]-2-(4-fluorophenyl)acetamide (100 mg) in toluene (2.4 mL) and NMP (1.3 mL) was added (4-bromo-3-methoxyphenyl)(morpholin-4-yl)methanone (119 mg), chloro(2-dicyclohexylphosphino-2′,4′,6′-tri-iso-propyl-1,1′-biphenyl)[2-(2-aminoethyl)phenyl]palladium(II) methyl-tert-butylether adduct (21.9 mg) and X-Phos (12.9 mg) and the flask was degassed twice and backfilled with argon. The mixture was stirred for 5 minutes at room temperature. Powdered potassium phosphate (281 mg) was added and the flask was degassed twice and backfilled with argon. The mixture was heated to reflux for 3 h. Further chloro(2-dicyclohexylphosphino-2′,4′,6′-tri-iso-propyl-1,1′-biphenyl)[2-(2-aminoethyl)phenyl]palladium(II) methyl-tert-butylether adduct (11 mg) and X-Phos (6.5 mg) were added and the flask was degassed twice and backfilled with argon. The mixture was heated to reflux for further 2 h. The reaction mixture was filtered through an aminophase-silica-gel column and the solvent was removed in vacuum. Aminophase-silica-gel chromatography followed by preparative reverse phase HPLC gave a solid that was triturated with dichloromethane to give 7 mg of the title compound.

¹H-NMR (300 MHz, DMSO-d₆), δ [ppm]=3.44-3.66 (10H), 3.89 (3H), 7.00-7.19 (4H), 7.34 (2H), 7.51-7.72 (6H), 8.07 (1H), 8.62 (1H), 10.03 (1H), 10.20 (1H).

### Example 4.2

### 2-(4-fluorophenyl)-N-[4-(2-{[2-methoxy-4-(methylsulfonyl)phenyl]amino}-1,3-benzothiazol-6-yl)phenyl]acetamide

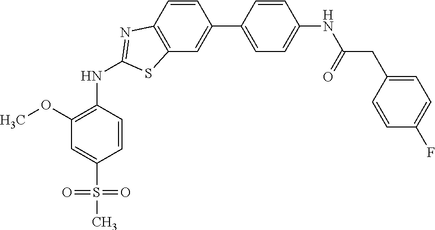 [SMILES: COc1cc(S(C)(=O)=O)ccc1Nc1nc2ccc(-c3ccc(NC(=O)Cc4ccc(F)cc4)cc3)cc2s1]

To a stirred suspension of N-[4-(2-amino-1,3-benzothiazol-6-yl)phenyl]-2-(4-fluorophenyl)acetamide (100 mg) in toluene (2.4 mL) and NMP (1.3 mL) was added 1-bromo-2-methoxy-4-(methylsulfonyl)benzene (105 mg), chloro(2-dicyclohexyl-phosphino-2′,4′,6′-tri-iso-propyl-1,1′-biphenyl)[2-(2-aminoethyl)phenyl]palladium(II) methyl-tert-butylether adduct (21.9 mg) and X-Phos (12.9 mg) and the flask was degassed twice and backfilled with argon. The mixture was stirred for 5 minutes at room temperature. Powdered potassium phosphate (281 mg) was added and the flask was degassed twice and backfilled with argon. The mixture was heated to reflux for 3 h. The reaction mixture was filtered through an aminophase-silica-gel column and the solvent was removed in vacuum. Aminophase-silica-get chromatography followed by preparative reverse phase HPLC gave a solid that was triturated with dichloromethane to give 25 mg of the title compound.

¹H-NMR (400 MHz, DMSO-d₆), δ [ppm]=3.18 (3H), 3.63 (2H), 3.98 (3H), 7.09-7.17 (2H), 7.29-7.39 (2H), 7.46 (1H), 7.55 (1H), 7.57-7.70 (6H), 8.12 (1H), 8.88 (1H), 10.24 (1H), 10.33 (1H).

### Example 4.3

### N-[4-(2-amino-1,3-benzothiazol-6-yl)phenyl]-2-(4-fluorophenyl)acetamide

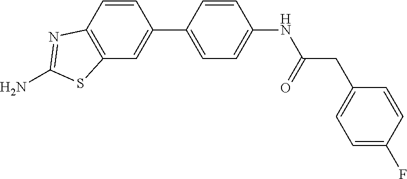 [SMILES: Nc1nc2ccc(-c3ccc(NC(=O)Cc4ccc(F)cc4)cc3)cc2s1]

To a stirred solution of 6-(4-aminophenyl)-1,3-benzothiazol-2-amine (645 mg) in THF (33 mL) was added Hünig base (0.50 mL), (4-fluorophenyl)acetic acid (454 mg) and HATU (1.12 g) and the mixture was stirred at room temperature for 16 h. Water was added, the mixture was stirred for 1 h and the mixture was extracted with ethyl acetate. The organic phase was washed with saturated sodium chloride solution, dried (sodium sulfate) and the solvent was removed in vacuum. The residue was triturated with dichloromethane to give 970 mg of the title compound.

¹H-NMR (400 MHz, DMSO-d₆), δ [ppm]=3.62 (2H), 7.08-7.16 (2H), 7.29-7.37 (3H), 7.45 (1H), 7.48 (2H), 7.53-7.59 (2H), 7.60-7.64 (2H), 7.90 (1H), 10.20 (1H).

### Example 5.1

### (2R)-2-(4-fluorophenyl)-N-[4-(2-{[2-methoxy-4-(methylsulfonyl)phenyl]amino}[1,2,4]triazolo[1,5-a]pyridin-7-yl)phenyl]propanamide

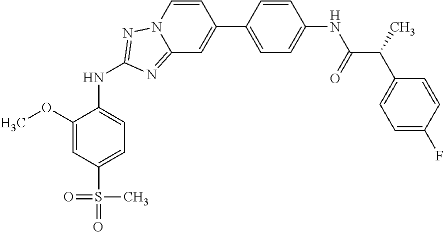 [SMILES: COc1cc(S(C)(=O)=O)ccc1Nc1nc2cc(-c3ccc(NC(=O)[C@H](C)c4ccc(F)cc4)cc3)ccn2n1]

To a stirred suspension of (2R)—N-[4-(2-amino[1,2,4]triazolo[1,5-a]pyridin-7-yl)phenyl]-2-(4-fluorophenyl)propanamide (100 mg) in toluene (4 mL) and NMP (0.2 mL) was added 1-bromo-2-methoxy-4-(methylsulfonyl)benzene (106 mg), chloro(2-dicyclohexylphosphino-2′,4′,6′-tri-iso-propyl-1,1′-biphenyl)[2-(2-aminoethyl)phenyl]palladium(II) methyl-tert-butylether adduct (22 mg), X-Phos (13 mg) and powdered potassium phosphate monohydrate (283 mg) and the flask was degassed twice and backfilled with argon. The mixture was heated to reflux for 16 h. The mixture was filtered and concentrated in vacuum. Silicagel chromatography followed by preparative reverse phase HPLC gave 10 mg of the title compound.

¹H-NMR (400 MHz, DMSO-d₆), δ [ppm]=1.44 (3H), 3.20 (3H), 3.88 (1H), 4.00 (3H), 7.12-7.24 (2H), 7.40-7.50 (4H), 7.56 (1H), 7.75 (2H), 7.86 (2H), 7.92 (1H), 8.52 (1H), 8.63 (1H), 8.86 (1H), 10.28 (1H).

LC-MS (Method 2): Rₜ=1.28 min; MS (ESIpos) m/z=560 [M+H]⁺.

### Example 5.2

### (2R)—N-{4-[2-({4-[(3-fluoroazetidin-1-yl)carbonyl]-2-methoxyphenyl}amino)[1,2,4]triazolo[1,5-a]pyridin-7-yl]phenyl}-2-(4-fluorophenyl)propanamide

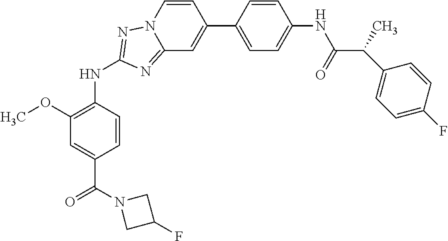 [SMILES: COc1cc(C(=O)N2CC(F)C2)ccc1Nc1nc2cc(-c3ccc(NC(=O)[C@H](C)c4ccc(F)cc4)cc3)ccn2n1]

To a stirred suspension of {4-[(7-chloro[1,2,4]triazolo[1,5-a]pyridin-2-yl)amino]-3-methoxyphenyl}(3-fluoroazetidin-1-yl)methanone (110 mg) in toluene (4.0 mL) and NMP (0.4 mL) was added (4-{[(2R)-2-(4-fluorophenyl)propanoyl]amino}phenyl)boronic acid (126 mg), powdered potassium phosphate monohydrate (248 mg), dicyclohexyl(2′,6′-dimethoxybiphenyl-2-yl)phosphine (24 mg) and Pd(OAc)₂ (6.6 mg) and the flask was degassed twice and backfilled with argon. The mixture was heated to reflux for 2 h. The reaction mixture was filtered and the solvent was removed in vacuum. Aminophase silicagel chromatography gave a solid that was triturated with ether to give 150 mg of the title compound.

¹H-NMR (400 MHz, DMSO-d₆), δ [ppm]=1.44 (3H), 3.82-3.98 (4H), 3.98-4.77 (4H), 5.31-5.59 (1H), 7.18 (2H), 7.24-7.35 (2H), 7.37-7.50 (3H), 7.75 (2H), 7.80-7.95 (3H), 8.29-8.48 (2H), 8.83 (1H), 10.27 (1H).

LC-MS (Method 2): Rₜ=1.27 min; MS (ESIpos) m/z=583 [M+H]⁺.

### Example 5.3

### (2R)—N-{4-[2-({4-[(3-fluoroazetidin-1-yl)carbonyl]-2-(2,2,2-trifluoroethoxy)phenyl}amino)[1,2,4]triazolo[1,5-a]pyridin-7-yl]phenyl}-2-(4-fluorophenyl)propanamide

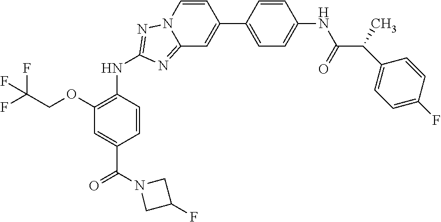 [SMILES: C[C@@H](C(=O)Nc1ccc(-c2ccn3nc(Nc4ccc(C(=O)N5CC(F)C5)cc4OCC(F)(F)F)nc3c2)cc1)c1ccc(F)cc1]

Starting from {4-[(7-chloro[1,2,4]triazolo[1,5-a]pyridin-2-yl)amino]-3-(2,2,2-trifluoroethoxy)phenyl}(3-fluoroazetidin-1-yl)methanone (70 mg) and (4-{[(2R)-2-(4-fluorophenyl)propanoyl]amino}phenyl)boronic acid (61 mg), Example 5.3. was prepared analogously to the procedure for the preparation of Example 5.2. Yield: 73 mg of the title compound.

¹H-NMR (400 MHz, DMSO-d₆), δ [ppm]=1.44 (3H), 3.89 (1H), 3.96-4.76 (4H), 4.96 (2H), 5.34-5.59 (1H), 7.13-7.22 (2H), 7.39-7.48 (5H), 7.75 (2H), 7.81-7.87 (2H), 7.89 (1H), 8.28 (1H), 8.38-8.44 (1H), 8.84 (1H), 10.28 (1H).

LC-MS (Method 2): Rₜ=1.35 min; MS (ESIpos) m/z=651 [M+H]⁺.

### Example 5.4

### (2R)-2-(4-fluorophenyl)-N-(4-{2-[(6-methoxy-1,1-dioxido-2, 3-dihydro-1-benzothiophen-5-yl)amino][1,2,4]triazolo[1,5-a]pyridin-7-yl}phenyl)propanamide

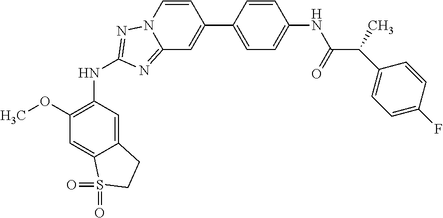 [SMILES: COc1cc2c(cc1Nc1nc3cc(-c4ccc(NC(=O)[C@H](C)c5ccc(F)cc5)cc4)ccn3n1)CCS2(=O)=O]

The compound of Example 5.4. can be prepared in analogy to the methods described herein.

### Example 5.5

### (2R)-2-(4-fluorophenyl)-N-[4-(2-{[4-(methylsulfonyl)-2-(2,2,2-trifluoroethoxy)phenyl]amino}[1,2,4]triazolo[1,5-a]pyridin-7-yl)phenyl]propanamide

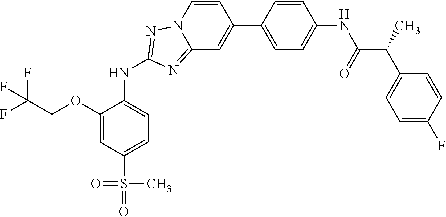 [SMILES: C[C@@H](C(=O)Nc1ccc(-c2ccn3nc(Nc4ccc(S(C)(=O)=O)cc4OCC(F)(F)F)nc3c2)cc1)c1ccc(F)cc1]

Starting from 7-chloro-N-[4-(methylsulfonyl)-2-(2,2,2-trifluoroethoxy)phenyl][1,2,4]triazolo[1,5-a]pyridin-2-amine (50 mg) and (4-{[(2R)-2-(4-fluorophenyl)propanoyl]amino}phenyl)boronic acid (51 mg), Example 5.5. was prepared analogously to the procedure for the preparation of Example 5.2. Yield: 20 mg of the title compound.

¹H-NMR (400 MHz, DMSO-d₆), δ [ppm]=1.42 (3H), 3.19 (3H), 3.87 (1H), 5.02 (2H), 7.12-7.20 (2H), 7.39-7.46 (3H), 7.62-7.67 (2H), 7.74 (2H), 7.81-7.88 (2H), 7.91 (1H), 8.53 (1H), 8.60 (1H), 8.85 (1H), 10.27 (1H).

LC-MS (Method 2): Rₜ=1.35 min; MS (ESIpos) m/z=628 [M+H]⁺.

### Example 5.6

### (2R)—N-[4-(2-{[4-(azetidin-1-ylcarbonyl)-2-methoxyphenyl]amino}[1,2,4]triazolo[1,5-a]pyridin-7-yl)phenyl]-2-(4-fluorophenyl)propanamide

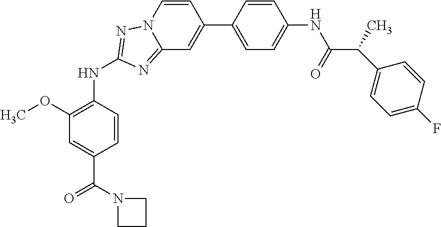 [SMILES: COc1cc(C(=O)N2CCC2)ccc1Nc1nc2cc(-c3ccc(NC(=O)[C@H](C)c4ccc(F)cc4)cc3)ccn2n1]

Starting from azetidin-1-yl{4-[(7-chloro[1,2,4]triazolo[1,5-a]pyridin-2-yl)amino]-3-methoxyphenyl}methanone (120 mg) and (4-{[(2R)-2-(4-fluorophenyl)propanoyl]amino}phenyl)boronic acid (144 mg), Example 5.6. was prepared analogously to the procedure for the preparation of Example 5.2.

Yield: 30 mg of the title compound.

¹H-NMR (400 MHz, DMSO-d₆), δ [ppm]=1.42 (3H), 2.25 (2H), 3.82-3.94 (4H), 4.03 (2H), 4.36 (2H), 7.12-7.20 (2H), 7.22-7.29 (2H), 7.35-7.46 (3H), 7.73 (2H), 7.80-7.89 (3H), 8.29 (1H), 8.33 (1H), 8.81 (1H), 10.26 (1H).

LC-MS (Method 2): Rₜ=1.27 min; MS (ESIpos) m/z=565 [M+H]⁺.

### Example 6.1

### (2R)-2-(4-fluorophenyl)-N-[4-(2-{[2-methoxy-4-(methylsulfonyl)phenyl]amino}imidazo[1,2-b]pyridazin-6-yl)phenyl]propanamide

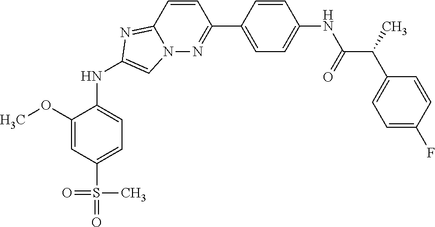 [SMILES: COc1cc(S(C)(=O)=O)ccc1Nc1cn2nc(-c3ccc(NC(=O)[C@H](C)c4ccc(F)cc4)cc3)ccc2n1]

To a stirred suspension of 6-chloro-N-[2-methoxy-4-(methylsulfonyl)phenyl]imidazo[1,2-b]pyridazin-2-amine (100 mg) in toluene (4.0 mL) and NMP (0.4 mL) was added (4-{[(2R)-2-(4-fluorophenyl)propanoyl]amino}phenyl)boronic acid (122 mg), powdered potassium phosphate monohydrate (240 mg), dicyclohexyl(2′,6′-dimethoxybiphenyl-2-yl)phosphine (23 mg) and Pd(OAc)₂ (6.4 mg) and the flask was degassed twice and backfilled with argon. The mixture was heated to reflux for 2 h. The reaction mixture was filtered and the solvent was removed in vacuum. Silicagel chromatography followed by aminophase silicagel chromatography and by preparative reverse phase HPLC gave a solid that was triturated with warm ethanol to give 35 mg of the title compound.

¹H-NMR (400 MHz, DMSO-d₆), δ [ppm]=1.44 (3H), 3.17 (3H), 3.89 (1H), 4.01 (3H), 7.12-7.24 (2H), 7.38-7.53 (4H), 7.70 (1H), 7.77 (2H), 7.97-8.08 (4H), 8.57 (1H), 8.84 (1H), 10.31 (1H).

LC-MS (Method 2): Rₜ=1.28 min; MS (ESIpos) m/z=560 [M+H]⁺.

### Example 7.1

### (2R)-2-(4-fluorophenyl)-N-[4-(2-{[3-methoxy-5-(methylsulfonyl)pyridin-2-yl]amino}imidazo[1, 2-a]pyridin-6-yl)phenyl]propanamide

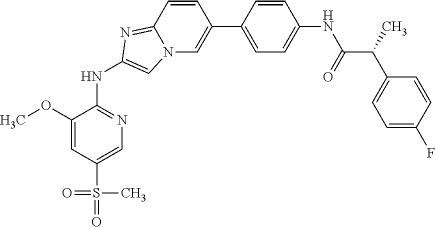 [SMILES: COc1cc(S(C)(=O)=O)cnc1Nc1cn2cc(-c3ccc(NC(=O)[C@H](C)c4ccc(F)cc4)cc3)ccc2n1]

A stirred suspension of 6-bromo-N-[3-methoxy-5-(methylsulfonyl)pyridin-2-yl]imidazo[1,2-a]pyridin-2-amine (70 mg), (4-{[2-(4-fluorophenyl)propanoyl]amino}phenyl)boronic acid (56 mg) and [1,1′-bis(diphenylphosphino)ferrocene]dichloropalladium(II) (14 mg) in 1,2-dimethoxyethane (1.29 mL) and an aqueous solution of potassium carbonate (2M, 0.26 mL) was stirred at 90° C. under argon overnight. After cooling, the mixture was diluted with water and extracted with ethyl acetate (3×). The combined organic phases were dried (MgSO₄), filtered and concentrated. The residue was purified by preparative reverse phase HPLC to give 20 mg of the title compound.

¹H-NMR (400 MHz, DMSO-d₆), δ [ppm]=1.43 (3H), 3.26 (3H), 3.87 (1H), 4.01 (3H), 7.13-7.20 (2H), 7.42-7.46 (2H), 7.47-7.50 (1H), 7.53-7.57 (2H), 7.64 (2H), 7.69-7.72 (2H), 8.32 (1H), 8.42 (1H), 8.92 (1H), 8.99 (1H), 10.18 (1H).

Further, the compounds of formula (I) of the present invention can be converted to any salt as described herein, by any method which is known to the person skilled in the art. Similarly, any salt of a compound of formula (I) of the present invention can be converted into the free compound, by any method which is known to the person skilled in the art.

### Biological Assay: Proliferation Assay

Cultivated tumor cells (MCF7, hormone dependent human mammary carcinoma cells, ATCC HTB22; NCI-H460, human non-small cell lung carcinoma cells, ATCC HTB-177; DU 145, hormone-independent human prostate carcinoma cells, ATCC HTB-81; HeLa-MaTu, human cervical carcinoma cells, EPO-GmbH, Berlin; HeLa-MaTu-ADR, multidrug-resistant human cervical carcinoma cells, EPO-GmbH, Berlin; HeLa human cervical tumor cells, ATCC CCL-2; B16F10 mouse melanoma cells, ATCC CRL-6475) were plated at a density of 5000 cells/well (MCF7, DU145, HeLa-MaTu-ADR), 3000 cells/well (NCI-H460, HeLa-MaTu, HeLa), or 1000 cells/well (B16F10) in a 96-well multititer plate in 200 μl of their respective growth medium supplemented 10% fetal calf serum. After 24 hours, the cells of one plate (zero-point plate) were stained with crystal violet (see below), while the medium of the other plates was replaced by fresh culture medium (200 μl), to which the test substances were added in various concentrations (0 μM, as well as in the range of 0.01-30 μM; the final concentration of the solvent dimethyl sulfoxide was 0.5%). The cells were incubated for 4 days in the presence of test substances. Cell proliferation was determined by staining the cells with crystal violet: the cells were fixed by adding 20 μl/measuring point of an 11% glutaric aldehyde solution for 15 minutes at room temperature. After three washing cycles of the fixed cells with water, the plates were dried at room temperature. The cells were stained by adding 100 μl/measuring point of a 0.1% crystal violet solution (pH 3.0). After three washing cycles of the stained cells with water, the plates were dried at room temperature. The dye was dissolved by adding 100 μl/measuring point of a 10% acetic acid solution. The extinction was determined by photometry at a wavelength of 595 nm. The change of cell number, in percent, was calculated by normalization of the measured values to the extinction values of the zero-point plate (=0%) and the extinction of the untreated (0 μm) cells (=100%). The IC50 values were determined by means of a 4 parameter fit using the company's own software.

The compounds of the present invention are characterized by the following IC₅₀ values, determined in a HeLa cell proliferation assay (as described above):

| Example | Inhibition of cell proliferation, | |
|---|---|---|
| Number | cell Line: HeLa IC₅₀ | |
| 5.1 | 118 | nM |
| 5.2 | 24 | nM |
| 5.3 | 13 | nM |
| 5.5 | 82 | nM |
| 5.6 | 57 | nM |
| 6.1 | 501 | nM |

### Mps-1 Kinase Assay

The human kinase Mps-1 phosphorylates a biotinylated substrate peptide. Detection of the phosphorylated product is achieved by time-resolved fluorescence resonance energy transfer (TR-FRET) from Europium-labelled anti-phospho-Serine/Threonine antibody as donor to streptavidin Labelled with cross-linked allophycocyanin (SA-XLent) as acceptor. Compounds are tested for their inhibition of the kinase activity.

N-terminally GST-tagged human full length recombinant Mps-1 kinase (purchased from Invitrogen, Karslruhe, Germany, cat. no PV4071) was used. As substrate for the kinase reaction a biotinylated peptide of the amino-acid sequence PWDPDDADITEILG (C-terminus in amide form, purchased from Biosynthan GmbH, Berlin) was used.

For the assay 50 nl of a 100-fold concentrated solution of the test compound in DMSO was pipetted into a black low volume 384 well microtiter plate (Greiner Bio-One, Frickenhausen, Germany), 2 μl of a solution of Mps-1 in assay buffer [0.1 mM sodium-ortho-vanadate, 10 mM MgCl₂, 2 mM DTT, 25 mM Hepes pH 7.7, 0.05% BSA, 0.001% Pluronic F-127] were added and the mixture was incubated for 15 min at 22° C. to allow pre-binding of the test compounds to Mps-1 before the start of the kinase reaction. Then the kinase reaction was started by the addition of 3 μl of a solution of 16.7 adenosine-tri-phosphate (ATP, 16.7 μM=>final conc. in the 5 μl assay volume is 10 μM) and peptide substrate (1.67 μM=>final conc. in the 5 μl assay volume is 1 μM) in assay buffer and the resulting mixture was incubated for a reaction time of 60 min at 22° C. The concentration of Mps-1 in the assay was adjusted to the activity of the enzyme lot and was chosen appropriate to have the assay in the linear range, typical enzyme concentrations were in the range of about 1 nM (final conc. in the 5 μl assay volume). The reaction was stopped by the addition of 3 μl of a solution of HTRF detection reagents (100 mM Hepes pH 7.4, 0.1% BSA, 40 mM EDTA, 140 nM Streptavidin-XLent [#61GSTXLB, Fa. Cis Biointernational, Marcoule, France], 1.5 nM anti-phospho(Ser/Thr)-Europium-antibody [#AD0180, PerkinElmer LAS, Rodgau-Jügesheim, Germany].

The resulting mixture was incubated 1 h at 22° C. to allow the binding of the phosphorylated peptide to the anti-phospho(Ser/Thr)-Europium-antibody. Subsequently the amount of phosphorylated substrate was evaluated by measurement of the resonance energy transfer from the Europium-labelled anti-phospho(Ser/Thr) antibody to the Streptavidin-XLent. Therefore, the fluorescence emissions at 620 nm and 665 nm after excitation at 350 nm was measured in a Viewlux TR-FRET reader (PerkinElmer LAS, Rodgau-Jügesheim, Germany). The “blank-corrected normalized ratio” (a Viewlux specific readout, similar to the traditional ratio of the emissions at 665 nm and at 622 nm, in which blank and Eu-donor crosstalk are subtracted from the 665 nm signal before the ratio is calculated) was taken as the measure for the amount of phosphorylated substrate. The data were normalised (enzyme reaction without inhibitor=0% inhibition, all other assay components but no enzyme=100% inhibition). Test compounds were tested on the same microtiter plate at 10 different concentrations in the range of 20 μM to 1 nM (20 μM, 6.7 μM, 2.2 μM, 0.74 μM, 0.25 μM, 82 nM, 27 nM, 9.2 nM, 3.1 nM and 1 nM, dilution series prepared before the assay at the level of the 100 fold conc. stock solutions by serial 1:3 dilutions) in duplicate values for each concentration and IC₅₀ values were calculated by a 4 parameter fit using an inhouse software.

| | Mps-1 Inhibition, | |
|---|---|---|
| | IC50 | |
| | (Assay with 10 μM | |
| Example No. | ATP) | |
| 1.1 | ≦1 | nM |
| 1.2 | ≦1 | nM |
| 1.3 | ≦1 | nM |
| 2.1 | ≦1 | nM |
| 2.2 | ≦1 | nM |
| 2.3 | 2.6 | nM |
| 2.4 | ≦1 | nM |
| 2.5 | ≦1 | nM |
| 2.6 | 2.1 | nM |
| 2.7 | 2.9 | nM |
| 2.8 | ≦1 | nM |
| 3.1 | 33.5 | nM |
| 3.2 | 22.4 | nM |
| 3.3 | 84.1 | nM |
| 3.4 | 206 | nM |
| 3.5 | 35.4 | nM |
| 3.6 | 14.6 | nM |
| 3.7 | 443 | nM |
| 3.8 | 71.6 | nM |
| 3.9 | 69 | nM |
| 4.1 | ≦1 | nM |
| 4.2 | 2.9 | nM |
| 4.3 | 8.6 | nM |
| 5.1 | ≦1 | nM |
| 5.2 | ≦1 | nM |
| 5.3 | ≦1 | nM |
| 5.5 | ≦1 | nM |
| 5.6 | ≦1 | nM |
| 6.1 | ≦1 | nM |
| 7.1 | ≦1 | nM |

### Spindle Assembly Checkpoint Assay

The spindle assembly checkpoint assures the proper segregation of chromosomes during mitosis. Upon entry into mitosis, chromosomes begin to condensate which is accompanied by the phosphorylation of histone H3 on serine 10. Dephosphorylation of histone H3 on serine 10 begins in anaphase and ends at early telophase. Accordingly, phosphorylation of histone H3 on serine 10 can be utilized as a marker of cells in mitosis. Nocodazole is a microtubule destabilizing substance. Thus, nocodazole interferes with microtubule dynamics and mobilises the spindle assembly checkpoint. The cells arrest in mitosis at G2/M transition and exhibit phosphorylated histone H3 on serine 10. An inhibition of the spindle assembly checkpoint by Mps-1 inhibitors overrides the mitotic blockage in the presence of nocodazole, and the cells complete mitosis prematurely. This alteration is detected by the decrease of cells with phosphorylation of histone H3 on serine 10. This decline is used as a marker to determine the capability of compounds of the present invention to induce a mitotic breakthrough.

Cultivated cells of the human cervical tumor cell line HeLa (ATCC CCL-2) were plated at a density of 2500 cells/well in a 384-well microtiter plate in 20 μl Dulbeco's Medium (w/o phenol red, w/o sodium pyruvate, w 1000 mg/mL glucose, w pyridoxine) supplemented with 1% (v/v) glutamine, 1% (v/v) penicillin, 1% (v/v) streptomycin and 10% (v/v) fetal calf serum. After incubation overnight at 37° C., 10 μl/well nocodazole at a final concentration of 0.1 μg/mL were added to cells. After 24 h incubation, cells were arrested at G2/M phase of the cell cycle progression. Test compounds solubilised in dimethyl sulfoxide (DMSO) were added at various concentrations (0 μM, as well as in the range of 0.005 μM-10 μM; the final concentration of the solvent DMSO was 0.5% (v/v)). Cells were incubated for 4 h at 37° C. in the presence of test compounds. Thereafter, cells were fixed in 4% (v/v) paraformaldehyde in phosphate buffered saline (PBS) at 4° C. overnight then permeabilised in 0.1% (v/v) Triton X™ 100 in PBS at room temperature for 20 min and blocked in 0.5% (v/v) bovine serum albumin (BSA) in PBS at room temperature for 15 min. After washing with PBS, 20 μl/well antibody solution (anti-phospho-histone H3 clone 3H10, FITC; Upstate, Cat#16-222; 1:200 dilution) was added to cells, which were incubated for 2 h at room temperature. Afterwards, cells were washed with PBS and 20 μl/well HOECHST 33342 dye solution (5 μg/mL) was added to cells and cells were incubated 12 min at room temperature in the dark. Cells were washed twice with PBS then covered with PBS and stored at 4° C. until analysis. Images were acquired with a Perkin Elmer OPERA™ High-Content Analysis reader. Images were analyzed with image analysis software MetaXpress™ from Molecular devices utilizing the Cell Cycle application module. In this assay both Labels HOECHST 33342 and phosphorylated Histone H3 on serine 10 were measured. HOECHST 33342 Labels DNA and is used to count cell number. The staining of phosphorylated Histone H3 on serine 10 determines the number of mitotic cells. Inhibition of Mps-1 decreases the number of mitotic cells in the presence of nocodazole indicating an inappropriate mitotic progression. The raw assay data were further analysed by four parameter logistic regression analysis to determine the IC₅₀ value for each tested compound.

Thus the compounds of the present invention effectively inhibit Mps-1 kinase and are therefore suitable for the treatment or prophylaxis of diseases of uncontrolled cell growth, proliferation and/or survival, inappropriate cellular immune responses, or inappropriate cellular inflammatory responses, particularly in which the uncontrolled cell growth, proliferation and/or survival, inappropriate cellular immune responses, or inappropriate cellular inflammatory responses is mediated by Mps-1, more particularly in which the diseases of uncontrolled cell growth, proliferation and/or survival, inappropriate cellular immune responses, or inappropriate cellular inflammatory responses are haemotological tumours, solid tumours and/or metastases thereof, e.g. Leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof.

1. A compound of formula (I): 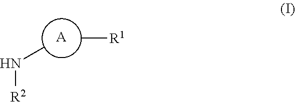
in which:
A is selected from: 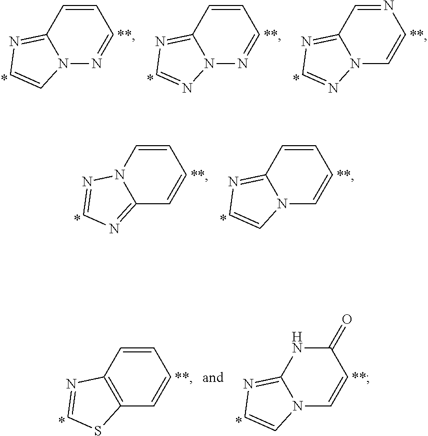
wherein * represents the point of attachment to the nitrogen atom and ** represents the point of attachment to the R¹ group;
R¹ represents a phenyl-group
which is substituted, one or more times, identically or differently, with a substituent selected from:
—OH, —N(H)C(═O)R⁶, —N(R⁷)C(═O)R⁶, —N(H)C(═O)NR⁶R⁷, —N(R⁷)C(═O)NR⁶R⁷, —NH₂, —NR⁶R⁷, —C(═O)N(H)R⁶, and —C(═O)NR⁶R⁷;
and
which is optionally substituted, one or more times, identically or differently, with a C₁-C₆-alkyl-group;
R² represents a hydrogen atom or a group selected from phenyl-, and pyridyl-;
said group being substituted, one or more times, identically or differently, with a substituent selected from:
halo-, hydroxy-, cyano-, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-,
R⁹—, R⁹—(C₁-C₆-alkyl)-, R⁹—(CH₂)ₙ(CHOH)(CH₂)ₘ—, R⁹—(C₁-C₆-alkoxy)-, R⁹—(CH₂)ₙ(CHOH)(CH₂)ₚ—O—, R⁹—(C₁-C₆-alkoxy-C₁-C₆-alkyl)-, R⁹—(C₁-C₆-alkoxy-C₁-C₆-alkyl)-O—, —O—(CH₂)ₙ—C(═O)NR⁹R⁷, R⁹—O—, —C(═O)R⁹, —C(═O)O—R⁹, —OC(═O)—R⁹, —N(H)C(═O)R⁹, —N(R⁷)C(═O)R⁹, —N(H)C(═O)NR⁹R⁷, —N(R⁷)C(═O)NR⁹R⁷, —NR⁹R⁷, —C(═O)N(H)R⁹, —C(═O)NR⁹R⁷, R⁹—S—, R⁹—S(═O)—, R⁹—, —S(═O)₂—, —N(H)S(═O)R⁹, —N(R⁷)S(═O)R⁹, —S(═O)N(H)R⁹, —S(═O)NR⁹R⁷, —N(H)S(═O)₂R⁹, —N(R⁷)S(═O)₂R⁹, —S(═O)₂N(H)R⁹, —S(═O)₂NR⁹R⁷, —S(═O)(═NR⁹)R⁷, —S(═O)(═NR⁷)R⁹ and —N═S(═O)(R⁹)R⁷;
or
R² represents a group selected from: 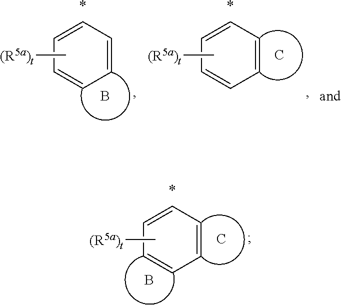
wherein * indicates the point of attachment of said group with the rest of the molecule;
B represents a 4- to 6-membered heterocyclic ring; which is optionally substituted, one or more times, identically or differently, with halo-, —CN, —OH, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R⁸—(C₁-C₆-alkoxy)-, R⁸—O—, —NR⁸R⁷, R⁸—S—, R⁸—S(═O)—, R⁸—S(═O)₂—, or (C₃-C₆-cycloalkyl)-(CH₂)ₙ—O—;
C represents a 4- to 6-membered heterocyclic ring; which is optionally substituted, one or more times, identically or differently, with halo-, —CN, —OH, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R⁸—(C₁-C₆-alkoxy)-, R⁸—O—, —NR⁸R⁷, R⁸—S—, R⁸—S(═O)—, R⁸—S(═O)₂—, or (C₃-C₆-cycloalkyl)-(CH₂)ₙ—O—;
each R^{5a}
independently represents a group selected from:
halo-, cyano, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R⁸—(C₁-C₆-alkoxy)-, R⁸—O—, —NR⁸R⁷, R⁸—S—, R⁸—S(═O)—, R⁸—S(═O)₂—, and (C₃-C₆-cycloalkyl)-(CH₂)ₙ—O—;
R⁶ represents a group selected from:
C₁-C₆-alkyl-, C₃-C₆-cycloalkyl-, 3- to 10-membered heterocycloalkyl-, aryl-, heteroaryl-, —(CH₂)_{q}—(C₃-C₆-cycloalkyl), —(CH₂)_{q}-heteroaryl, —(CH₂)_{q}-(3- to 10-membered heterocycloalkyl), and —(CH₂)_{q}-aryl;
said group being optionally substituted, one or more times, identically or differently, with a substituent selected from:
halo-, hydroxy-, cyano-, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R⁸—(C₁-C₆-alkyl)-, R⁸—(CH₂)ₙ(CHOH)(CH₂)ₘ—, R⁸—(C₁-C₆-alkoxy)-, R⁸—(CH₂)ₙ(CHOH)(CH₂)ₚ—O—, R⁸—(C₁-C₆-alkoxy-C₁-C₆-alkyl)-, R⁸—(C₁-C₆-alkoxy-C₁-C₆-alkyl)-O—, aryl-, R⁸—O—, —C(═O)R⁸, —C(═O)O—R⁸, —OC(═O)—R⁸, —N(H)C(═O)R⁸, —N(R⁷)C(═O)R⁸, —N(H)C(═O)NR⁸R⁷, —N(R⁷)C(═O)NR⁸R⁷, —NR⁸R⁷, —C(═O)N(H)R⁸, —C(═O)NR⁸R⁷, R⁸—S—, R⁸—S(═O)—, R⁸ —S(═O)₂—, —N(H)S(═O)R⁸, —N(R⁷)S(═O)R⁸, —S(═O)N(H)R⁸, —S(═O)NR⁸R⁷, —N(H)S(═O)₂R⁸, —N(R⁷)S(═O)₂R⁸, —S(═O)₂N(H)R⁸, —S(═O)₂NR⁸R⁷, —S(═O)(═NR⁸)R⁷, —S(═O)(═NR⁷)R⁸, and —N═S(═O)(R⁸)R⁷;
R⁷ represents a hydrogen atom, a C₁-C₆-alkyl-, or C₃-C₆-cycloalkyl-group;
or
R⁶ and R⁷,
together with the nitrogen atom to which they are attached, represent a 3- to 10-membered heterocycloalkyl-group;
R⁸ represents a hydrogen atom, a C₁-C₆-alkyl- or C₃-C₆-cycloalkyl-group;
R⁹ represents a C₁-C₆-alkyl- or C₃-C₆-cycloalkyl-group;
or
R⁹ and R⁷,
together with the nitrogen atom to which they are attached, represent a 3- to 10-membered heterocycloalkyl-group, which is optionally substituted with halogen;
n, m, and p
represent, independently from each other, an integer of 0, 1, 2, 3, 4, or 5;
q represents an integer of 0, 1, 2 or 3;
and
t represents an integer of 0, 1 or 2;
or a stereoisomer, a tautomer, an N-oxide, a hydrate, a solvate, or a salt thereof, or a mixture of same.

2. The compound according to claim 1, wherein:
R¹ represents a phenyl group
which is substituted, one or more times, identically or differently, with a substituent selected from:
—OH, —N(H)C(═O)R⁶, —NH₂, and —C(═O)N(H)R⁶; and
which is optionally substituted, one or more times, identically or differently, with C₁-C₆-alkyl-; and
R⁶ represents a group selected from:
—CH₂—(C₃-C₆-cycloalkyl), and —CH₂-aryl;
wherein said group is optionally substituted, one or more times, identically or differently, with a substituent selected from:
halo-, C₁-C₆-alkyl-, halo-C₁-C₆-alkyl-, and halo-C₁-C₆-alkoxy-.

3. The compound according to claim 1, wherein:
R¹ represents 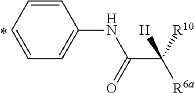
wherein * indicates the point of attachment of said group with the rest of the molecule;
R¹⁰ represents a group selected from: C₁-C₃-alkyl-, hydroxy-C₁-C₃-alkyl-, and N(H)(R⁸)—C₁-C₃-alkyl-; and
R^{6a} represents a 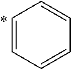
group;
wherein * indicates the point of attachment of said group with the rest of the molecule;
wherein said group is optionally substituted, one or more times, identically or differently, with a halogen atom or a methyl-group.

4. The compound according to claim 1, wherein:
R² represents 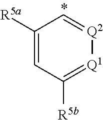
wherein * indicates the point of attachment of said group with the rest of the molecule;
R^{5a} represents a group selected from: C₁-C₄-alkoxy-, halo-C₁-C₄-alkoxy-, and C₁-C₄-alkyl;
R^{5b} represents a group selected from: —C(═O)N(H)R⁹, —C(═O)NR⁹R⁷, —NR⁹R⁷, and R⁹—S(═O)₂—;
Q¹ represents CH or N;
Q² represents CH or N;
with the proviso that Q¹ represents CH if Q² represents N, and Q² represents CH if Q¹ represents N.

5. The compound according to claim 1, wherein:
R² represents 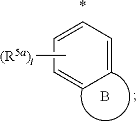
wherein * indicates the point of attachment of said group with the rest of the molecule;
B represents a 5- to 6-membered heterocyclic ring; which is optionally, one or more times, identically or differently, substituted with C₁-C₃-alkyl-, or halo-C₁-C₃-alkyl-;
t=1;
R^{5a} represents a group selected from:
halo-, C₁-C₆-alkyl-, C₁-C₆-alkoxy-, halo-C₁-C₆-alkoxy-, C₁-C₆-alkoxy-C₁-C₆-alkyl-, and
(C₃-C₆-cycloalkyl)-(CH₂)ₙ—O—; and
n=0 or 1.

6. The compound according to claim 1, wherein:
R⁶ represents —(CH₂)_{q}—(C₃-C₆-cycloalkyl) or —(CH₂)_{q}-aryl;
said group being optionally substituted, one or more times, identically or differently, with halo- or C₁-C₃-alkyl-;
and
q=0 or 1.

7. The compound according to claim 1, wherein:
R⁷ represents a hydrogen atom or a C₁-C₆-alkyl-group; and
R⁸ represents a C₁-C₆-alkyl-group.

8. The compound according to claim 1, wherein:
R⁹ and R⁷,
together with the nitrogen atom to which they are attached, represent a group selected from: 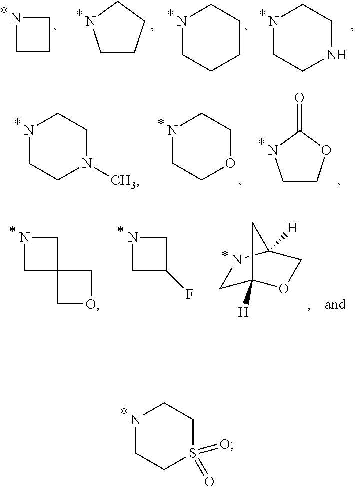
wherein * indicates the point of attachment of said group with the rest of the molecule.

9. The compound according to claim 1, wherein:
R⁹ represents a C₁-C₆-alkyl-group.

10. The compound according to claim 1, wherein:
A is selected from: 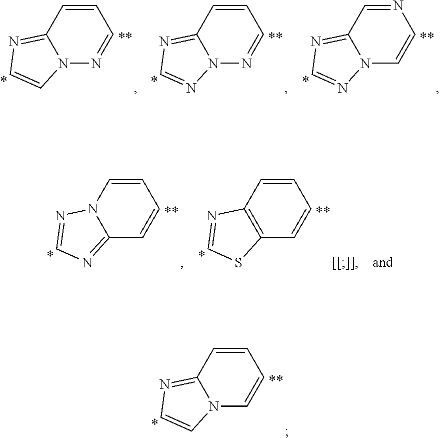
wherein * represents the point of attachment to the nitrogen atom and ** represents the point of attachment to the R¹ group.

11. The compound according to claim 1, wherein:
A represents 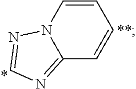
wherein * represents the point of attachment to the nitrogen atom and ** represents the point of attachment to the R¹ group.

12. The compound according to claim 1, which is selected from the group consisting of:
2-(4-fluorophenyl)-N-[4-(2-{[2-methoxy-4-(morpholin-4-ylcarbonyl)phenyl]amino}[1,2,4]triazolo[1,5-b]pyridazin-6-yl)phenyl]acetamide,
2-(4-fluorophenyl)-N-[4-(2-{[4-(2-hydroxypropan-2-yl)-2-methoxyphenyl]amino}[1,2,4]triazolo[1,5-b]pyridazin-6-yl)phenyl]acetamide,
2-(4-fluorophenyl)-N-[4-(2-{[2-methoxy-4-(methylsulfonyl)phenyl]amino}[1,2,4]triazolo[1,5-b]pyridazin-6-yl)phenyl]acetamide,
N-(4-{2-[(2-cyanophenyl)amino][1,2,4]triazolo[1,5-a]pyrazin-6-yl}phenyl)-2-(4-fluorophenyl)acetamide,
N-(4-{2-[(2-cyano-3-fluorophenyl)amino][1,2,4]triazolo[1,5-a]pyrazin-6-yl}phenyl)-2-(4-fluorophenyl)acetamide,
N-(4-{2-[(2-cyanophenyl)amino][1,2,4]triazolo[1,5-a]pyrazin-6-yl}phenyl)-2-phenylacetamide,
2-(4-fluorophenyl)-N-[4-(2-{[2-methoxy-4-(4-methylpiperazin-1-yl)phenyl]amino}[1,2,4]triazolo[1,5-a]pyrazin-6-yl)phenyl]acetamide,
N-(4-{2-[(2-cyano-3-fluorophenyl)amino][1,2,4]triazolo[1,5-a]pyrazin-6-yl}phenyl)-2-phenylacetamide,
N-(4-{2-[(2-cyanophenyl)amino][1,2,4]triazolo[1,5-a]pyrazin-6-yl}phenyl)-2-(3,4-difluorophenyl)acetamide,
N-(4-{2-[(2-cyano-3-fluorophenyl)amino][1,2,4]triazolo[1,5-a]pyrazin-6-yl}phenyl)-2-(3,4-difluorophenyl)acetamide,
2-(4-fluorophenyl)-N-[4-(2-{[2-methoxy-4-(methylsulfonyl)phenyl]amino}[1,2,4]triazolo[1,5-a]pyrazin-6-yl)phenyl]acetamide,
2-{[6-(4-hydroxy-3,5-dimethylphenyl)[1,2,4]triazolo[1,5-a]pyrazin-2-yl]amino}benzonitrile,
N-(4-{2-[(2-cyanophenyl)amino][1,2,4]triazolo[1,5-a]pyrazin-6-yl}phenyl)-2-cyclopropylacetamide,
3-{2-[(2-cyanophenyl)amino][1,2,4]triazolo[1,5-a]pyrazin-6-yl}-N-cyclopropylbenzamide,
3-{2-[(2-cyanophenyl)amino][1,2,4]triazolo[1,5-a]pyrazin-6-yl}-N-ethylbenzamide,
3-{2-[(2-cyanophenyl)amino][1,2,4]triazolo[1,5-a]pyrazin-6-yl}-N-cyclopentylbenzamide,
N-(4-{2-[(2-cyano-3-fluorophenyl)amino][1,2,4]triazolo[1,5-a]pyrazin-6-yl}phenyl)-2-cyclopropylacetamide,
2-{[6-(4-aminophenyl)[1,2,4]triazolo[1,5-a]pyrazin-2-yl]amino}benzonitrile,
4-{2-[(2-methoxyphenyl)amino][1,2,4]triazolo[1,5-a]pyrazin-6-yl}-2,6-dimethylphenol,
3-{2-[(2-cyanophenyl)amino][1,2,4]triazolo[1,5-a]pyrazin-6-yl}-N-cyclohexylbenzamide,
2-(4-fluorophenyl)-N-[4-(2-{[2-methoxy-4-(morpholin-4-ylcarbonyl)phenyl]amino}-1,3-benzothiazol-6-yl)phenyl]acetamide,
2-(4-fluorophenyl)-N-[4-(2-{[2-methoxy-4-(methylsulfonyl)phenyl]amino}-1,3-benzothiazol-6-yl)phenyl]acetamide,
N-[4-(2-amino-1,3-benzothiazol-6-yl)phenyl]-2-(4-fluorophenyl)acetamide,
(2R)-2-(4-fluorophenyl)-N-[4-(2-{[2-methoxy-4-(methylsulfonyl)phenyl]amino}[1,2,4]triazolo[1,5-a]pyridin-7-yl)phenyl]propanamide,
(2R)—N-{4-[2-({4-[(3-fluoroazetidin-1-yl)carbonyl]-2-methoxyphenyl}amino)[1,2,4]triazolo[1,5-a]pyridin-7-yl]phenyl}-2-(4-fluorophenyl)propanamide,
(2R)—N-{4-[2-({4-[(3-fluoroazetidin-1-yl)carbonyl]-2-(2,2,2-trifluoroethoxy)phenyl}amino)[1,2,4]triazolo[1,5-a]pyridin-7-yl]phenyl}-2-(4-fluorophenyl)propanamide,
(2R)-2-(4-fluorophenyl)-N-(4-{2-[(6-methoxy-1, 1-dioxido-2, 3-dihydro-1-benzothiophen-5-yl)amino][1,2,4]triazolo[1,5-a]pyridin-7-yl}phenyl)propanamide,
(2R)-2-(4-fluorophenyl)-N-[4-(2-{[4-(methylsulfonyl)-2-(2,2,2-trifluoroethoxy)phenyl]amino}[1,2,4]triazolo[1,5-a]pyridin-7-yl)phenyl]propanamide,
(2R)—N-[4-(2-{[4-(azetidin-1-ylcarbonyl)-2-methoxyphenyl]amino}[1,2,4]triazolo[1,5-a]pyridin-7-yl)phenyl]-2-(4-fluorophenyl)propanamide,
(2R)-2-(4-fluorophenyl)-N-[4-(2-{[2-methoxy-4-(methylsulfonyl)phenyl]amino}imidazo[1,2-b]pyridazin-6-yl)phenyl]propanamide and
(2R)-2-(4-fluorophenyl)-N-[4-(2-{[3-methoxy-5-(methylsulfonyl)pyridin-2-yl]amino}imidazo[1,2-a]pyridin-6-yl)phenyl]propanamide,
or a stereoisomer, a tautomer, an N-oxide, a hydrate, a solvate, or a salt thereof, or a mixture of same.

13. (canceled)

14. A pharmaceutical composition comprising a compound of formula (I), or a stereoisomer, a tautomer, an N-oxide, a hydrate, a solvate, or a pharmaceutically acceptable salt thereof, or a mixture of same, according to claim 1, and a pharmaceutically acceptable diluent or carrier.

15. A pharmaceutical combination comprising:
a compound of formula (I), or a stereoisomer, a tautomer, an N-oxide, a hydrate, a solvate, or a pharmaceutically acceptable salt thereof, or a mixture of same, according to claim 1;
and
one or more agents selected from: a taxane, Docetaxel, Paclitaxel, or Taxol; an epothilone, Ixabepilone, Patupilone, or Sagopilone; Mitoxantrone; Predinisolone; Dexamethasone; Estramustin; Vinblastin; Vincristin; Doxorubicin; Adriamycin; Idarubicin; Daunorubicin; Bleomycin; Etoposide; Cyclophosphamide; Ifosfamide; Procarbazine; Melphalan; 5-Fluorouracil; Capecitabine; Fludarabine; Cytarabine; Ara-C; 2-Chloro-2′-deoxyadenosine; Thioguanine; an anti-androgen, Flutamide, Cyproterone acetate, Bicalutamide; Bortezomib; a platinum derivative, Cisplatin, Carboplatin; Chlorambucil; Methotrexate; and Rituximab.

16. (canceled)

17. (canceled)

18. A method for the treatment of a disease of uncontrolled cell growth, proliferation or survival, an inappropriate cellular immune response, or an inappropriate cellular inflammatory response, comprising administering to a patient in need thereof a therapeutically effective amount of a compound of formula (I), or a stereoisomer, a tautomer, an N-oxide, a hydrate, a solvate, or a pharmaceutically acceptable salt thereof, or a mixture of same, according to claim 1.

19. The method according to claim 18, wherein the uncontrolled cell growth, proliferation or survival, inappropriate cellular immune response, or inappropriate cellular inflammatory response is mediated by Mps-1.

20. The method according to claim 19, wherein the disease of uncontrolled cell growth, proliferation or survival, inappropriate cellular immune response, or inappropriate cellular inflammatory response is a haemotological tumour, a solid tumour or metastases thereof.

21. The method according to claim 20, wherein the haemotological tumour, solid tumour or metastases thereof is selected from leukaemias and myelodysplastic syndrome, malignant lymphomas, head and neck tumours, brain tumours and brain metastases, tumours of the thorax, non-small cell and small cell lung tumours, gastrointestinal tumours, endocrine tumours, mammary and other gynaecological tumours, urological tumours, renal, bladder and prostate tumours, skin tumours, and sarcomas, or metastases thereof.


SEQUENCE LISTING (1 sequence)

SEQ ID NO: 1 - protein, 14 aa, Unknown
  biotinylated peptide of the amino-acid sequence PWDPDDADITEILG
PWDPDDADITEILG